(19) United States
(12) Patent Application Publication
(10) Pub. No.: US 2013/0323737 A1
(43) Pub. Date: Dec. 5, 2013

(54) METHOD AND SYSTEM FOR ANALYZING A SAMPLE

(71) Applicants: Frederic Zenhausern, Fountain Hills, AZ (US); Alan Nordquist, Payson, AZ (US); Ralf Lenigk, Chandler, AZ (US); Cedric Hurth, Tempe, AZ (US); Jianing Yang, Tempe, AZ (US); Xiaojia Chen, Chandler, AZ (US); Matthew Estes, Tempe, AZ (US); John Lee-Edghill, Birmingham West Midlands (GB); Nina Moran, Birmingham West Midlands (GB); Andrew Hopwood, Birmingham West Midlands (GB); Pieris Koumi, Birmingham West Midlands (GB)

(72) Inventors: Frederic Zenhausern, Fountain Hills, AZ (US); Alan Nordquist, Payson, AZ (US); Ralf Lenigk, Chandler, AZ (US); Cedric Hurth, Tempe, AZ (US); Jianing Yang, Tempe, AZ (US); Xiaojia Chen, Chandler, AZ (US); Matthew Estes, Tempe, AZ (US); John Lee-Edghill, Birmingham West Midlands (GB); Nina Moran, Birmingham West Midlands (GB); Andrew Hopwood, Birmingham West Midlands (GB); Pieris Koumi, Birmingham West Midlands (GB)

(73) Assignee: BIOACCEL, Phoenix, AZ (US)

(21) Appl. No.: 13/816,394

(22) PCT Filed: Aug. 10, 2011
(86) PCT No.: PCT/GB2011/051508

§ 371 Date: Aug. 23, 2013

Related U.S. Application Data
(60) Provisional application No. 61/372,330, filed on Aug. 10, 2010.

(51) Int. Cl. C12Q 1/68; G01N 27/447

(52) U.S. Cl. CPC C12Q 1/6846; G01N 27/447
USPC 435/6.12; 435/287.2; 204/450; 204/600

(57) ABSTRACT

A method and system for analysing a sample are provided, wherein one or more process steps and/or sample processors are provided separately from the instrument, for instance a sample receiving step and sample preparation step and sample extraction step and sample retention step and/or purification step and washing step and elution step, and one or more process steps and/or sample processors provided by the instrument as an integrated set, the one or more process steps and/or sample processors provided by the instrument including a sample receiving step and amplification step and denaturing step and investigation step and detection step and results analysis step and results output step. Other combinations of the split in location of the steps are possible. The optimisation of the split allows the accurate processing by a cartridge based instrument of the sample, whilst fully interfacing with a variety of sample collection and/or preparation approaches.

The invention concerns improvements in and relating to analysis, particularly, but not exclusively, in relation to biological samples.

According to a first aspect of the invention, there is provided an instrument for analysing a sample, the instrument including:

a device having one or more sample processors;

electronics for operating the sample processors.

According to a second aspect of the invention, there is provided a device, for processing a sample, the device including:

one or more sample processors.

According to a third aspect of the invention, there is provided a method of producing a device, the method including:

forming one or more sample processors;

providing electronics for operating the sample processors.

The instrument may provide some of a set of process steps and/or sample processors. One or more process steps and/or sample processors may be provided separately from the instrument. The device may provide some of a set of process steps and/or sample processors. One or more process steps and/or sample processors may be provided separately from the device. The process steps and/or sample processors may include a sample receiving step and/or sample preparation step and/or sample extraction step and/or sample retention step and/or purification step and/or washing step and/or elution step and/or further sample receiving step and/or amplification step and/or PCR step and/or denaturing step and/or investigation step and/or detection step and/or electrophoresis step and/or analysis step and/or results output step. The process steps and/or sample processors may include a sample receiving step and/or sample preparation step and/or sample extraction step and/or sample retention step and/or purification step and/or washing step and/or elution step provided separately from the instrument. The process steps and/or sample processors may include a sample receiving step and/or sample preparation step and/or sample extraction step and/or sample retention step and/or purification step and/or washing step and/or elution step provided separately from the device.

The instrument may provide an integrated set of process steps and/or sample processors. The process steps and/or sample processors may include a sample receiving step and/or sample preparation step and/or sample extraction step and/or sample retention step and/or purification step and/or washing step and/or elution step and/or further sample receiving step and/or amplification step and/or PCR step and/or denaturing step and/or investigation step and/or detection step and/or electrophoresis step and/or analysis step and/or results output step. Preferably the instrument may provide an integrated set of process steps and/or sample processors which include a further sample receiving step and/or amplification step and/or PCR step and/or denaturing step and/or investigation step and/or detection step and/or electrophoresis step and/or analysis step and/or results output step.

The device may be a cartridge. The device is preferably a single use device. The device is preferably only used to process and/or provide the results for one sample. The device is preferably disposable.

The device may have an orientation of use, for instance in the instrument.

The further sample receiving step may be provided on the device.

The further sample receiving step may include an inlet to the device. The further sample receiving step may include a chamber, preferably into which the sample is received. The chamber may have an inlet in the upper portion of the chamber, for instance the upper 20%. The chamber may have a gas outlet, for instance a vent. The gas outlet may be provided in the upper portion of the chamber, for instance the upper 20%.

The chamber may be connected to a pump, for instance an electrochemical pump. The pump may be a first pump provided on the device. The first pump may provide the drive to move one or more fluids and/or liquids through the chamber and/or one or more further chambers, for instance an amplification chamber and/or eleventh chamber.

The further sample receiving step may have a first state in which it is isolated from one of more of the other steps in the cartridge. The one or more other steps may be a sample amplification step and/or denaturation step and/or detection step and/or electrophoresis step and/or analysis step. The further sample receiving step may have the first state during loading of the sample. The further sample receiving step may be provided with a valve. The valve may be provided at the sample outlet and/or on the channel leading from the further sample receiving step and/or leading from the sample outlet. The valve may be a closed state to open state valve.

The outlet channel may be connected to the next step, for instance to the sample amplification step.

Particularly in embodiments where one or more of a sample receiving step and/or sample preparation step and/or sample extraction step and/or sample retention step and/or purification step and/or washing step and/or elution step are provided by the instrument and/or device, then the following features may individually and/or in combinations be provided.

The chamber may be connected to a pump, for instance an electrochemical pump. The pump may be a first pump provided on the device. The first pump may provide the drive to move one or more fluids and/or liquids through the chamber and/or second chamber and/or third chamber and/or fourth chamber and/or into a fifth chamber. The inlet from the pump may be provided in the upper section of the chamber, for instance the upper 20%, preferably upper 5%. The chamber may have a sample outlet. The sample outlet may be provided in the lower portion of the chamber, for instance the lower 20%, more preferably lower 10% and ideally the lowest part of the chamber. The outlet may be in the bottom wall of the chamber. The sample receiving step may have a first state in which it is isolated from one of more of the other steps in the cartridge. The one or more other steps may be a sample preparation step and/or sample extraction step and/or sample retention step and/or purification step and/or washing step and/or elution step and/or sample amplification step and/or denaturation step and/or detection step and/or electrophoresis step and/or analysis step. The sample receiving step may have the first state during loading of the sample. The sample receiving step may be provided with a valve. The valve may be provided at the sample outlet and/or on the channel leading from the sample receiving step and/or leading from the sample outlet. The valve may be a closed state to open state valve. The outlet channel may be connected to the next step, for instance to the sample preparation step. The sample preparation step may be provided on the device. The sample preparation step may be provided on the same device as the sample receiving step. The sample preparation step may include an inlet, preferably a channel. The channel may be connected to the sample receiving step. The sample preparation step may include a first chamber, preferably into which the sample passes. The first chamber may have an inlet in the upper portion of the first chamber, for instance the upper 20%. The first chamber may have a circular cross-section. The cross-section may be relative to a horizontal axis. The first chamber may be provided with a buffer. The buffer may be provided to control conditions for a subsequent process and/or reaction, for instance in one or more further chambers and/or channels. The first chamber may be provided with a one or more particles. The particles may be beads. One or more of the particles may be magnetic. The one or more particles may have a magnetic material within a surface layer or layers. The particles may be provided with one or more reagents or materials which releasable bind and/or link and/or combine with a part of the sample, for instance DNA. The first chamber may have a sample outlet. The sample outlet may be provided in the lower portion of the chamber, for instance the lower 20%, more preferably lower 10% and ideally the lowest part of the chamber. The outlet may be in the bottom wall of the chamber. References to vertical within the document may mean within 25° of the vertical, preferably within 10° and ideally within 5°, and potentially be completely vertical. References to horizontal within the document may mean within 25° of the horizontal, preferably within 10° and ideally within 5°, and potentially be completely horizontal. Reference to the number of a chamber, such as to a fourth chamber do not mean or imply that the chamber has to be preceded by such a number of chambers. The number is merely used to clarify one chamber from another. The sample outlet may connect to a channel. Preferably the channel has a plurality of sections. The channel may have a vertical section and/or a horizontal section and/or a second vertical section and/or a second horizontal section and/or a third vertical section. The channel may have a vertical section and a horizontal section and a second vertical section and/or a second horizontal section and/or a third vertical section. The channel may connect to a second chamber. The second chamber may have an inlet in the upper portion of the second chamber, for instance the upper 20%. The second chamber may have a elongate cross-section. The second chamber may have a cross-section formed by a semicircle at each end and a rectilinear section joining them. The cross-section may be relative to a horizontal axis. The second chamber may be provided with one or more particles. The particles may be beads. One or more of the particles may be magnetic. The one or more particles may have a magnetic material within a surface layer or layers. The particles may be provided with one or more reagents or materials which releasable bind and/or link and/or combine with a part of the sample, for instance DNA. The second chamber may be provided with a buffer. The buffer may be provided to control conditions for a subsequent process and/or reaction, for instance in one or more further chambers and/or channels. The second chamber may have a sample outlet. The sample outlet may be provided in the lower portion of the second chamber, for instance the lower 20%, more preferably lower 10% and ideally the lowest part of the second chamber. The outlet may be in the bottom wall of the chamber. The sample outlet may connect to a channel. Preferably the channel has a plurality of sections. The channel may have a vertical section and/or a horizontal section and/or a second vertical section and/or a second horizontal section and/or a third vertical section and/or a third horizontal section and/or fourth vertical section. The channel may have a vertical section and a horizontal section and a second vertical section and a second horizontal section and a third vertical section and/or a third horizontal section and/or fourth vertical section. The channel may connect to a third chamber. The third chamber may have an inlet in the upper portion of the third chamber, for instance the upper 25%. The third chamber may have a non-linear cross-section. The third chamber may have a cross-section formed by a semicircles or part semicircles at one or both ends. A rectilinear section may join the semicircles or part semicircle together. The cross-section may be relative to a horizontal axis. The third chamber may be provided with a one or more particles. The particles may be beads. One or more of the particles may be magnetic. The one or more particles may have a magnetic material within a surface layer or layers. The particles may be provided with one or more reagents or materials which releasable and/or reversibly bind and/or link and/or combine with a part of the sample, for instance DNA. The third chamber may have a gas outlet, for instance a vent. The gas outlet may be provided in the upper portion of the chamber, for instance the upper 20%, preferably upper 10% and ideally upper 5%. The gas outlet may be provided in the top wall of the third chamber. The gas outlet may be provided in a recess at the top of the third chamber. The gas outlet may lead to the outside of the device, for instance through a vent. A valve may be provided between the third chamber and the vent. The valve may be an open state to closed state valve. The third chamber may have a sample outlet. The sample outlet may be provided in the lower portion of the third chamber, for instance the lower 10%, more preferably lower 5% and ideally the lowest part of the third chamber. The outlet may be in the bottom wall of the chamber. The sample outlet may connect to a channel. Preferably the channel has a plurality of sections. The channel may have a vertical section and/or a horizontal section and/or a second vertical section and/or a second horizontal section and/or a third horizontal section and/or a third vertical section. The channel may have a vertical section and a horizontal section and a second vertical section and a second horizontal section and/or a third vertical section and/or a third horizontal section. The channel may connect to one or more further chambers, such as a fourth chamber. The sample preparation step or a part thereof may have a first state in which it is isolated from one of more of the other steps in the cartridge and/or from one or more other parts of the sample preparation step. The one or more other steps may be a sample receiving step and/or sample extraction step and/or sample retention step and/or purification step and/or washing step and/or elution step and/or sample amplification step and/or electrophoresis step and/or analysis step. The sample preparation step or part thereof may have the first state during contacting of the sample with the buffer and/or particles and/or first chamber and/or second chamber and/or third chamber. The sample preparation step or a part thereof may be provided with a valve. The valve may be provided at the sample outlet, preferably from the third chamber and/or on the channel leading from the sample preparation step to a further step and/or on the channel leading from the part of the sample preparation step to the next part of the sample preparation step and/or on the channel leading from the sample outlet. The valve may be a closed state to open state valve. The sample preparation step and/or a part of the sample preparation step, particularly the part that follows the part described above, may include a fourth chamber. The fourth chamber may have an inlet in the upper portion of the fourth chamber, for instance the upper 25%. The inlet may be in a corner of the fourth chamber. The fourth chamber may have a non-linear cross-section. The fourth chamber may have a cross-section formed by a horizontal top wall, horizontal or inclined lower wall and transition end walls joining the top and lower walls. The transition end walls may be curved. The cross-section may be relative to a horizontal axis. The fourth chamber may be provided with a gas, such as air, preferably prior to the sample arrival. The fourth chamber may have a sample outlet. The sample outlet may be provided in the lower portion of the fourth chamber, for instance the lower 10%, more preferably lower 5% and ideally the lowest part of the fourth chamber. The outlet may be in the bottom wall of the chamber or preferably in a corner of the chamber, ideally the corner opposing the inlet. The sample outlet may connect to a channel. Preferably the channel has a plurality of sections. The channel may have a vertical section and/or a horizontal section and/or a second vertical section. The channel may have a vertical section and a horizontal section and a second vertical section. The channel may connect to a fifth chamber. The fifth chamber may have an inlet in the upper portion of the fifth chamber, for instance the upper 25%. The inlet may be in a corner of the fifth chamber. The fifth chamber may have a non-linear cross-section. The fifth chamber may have a cross-section formed by a horizontal top wall, horizontal or inclined lower wall and transition end walls joining the top and lower walls. The transition end walls may be curved. The cross-section may be relative to a horizontal axis. The fifth chamber may be provided with a gas, such as air, preferably prior to the sample arrival. The fifth chamber may have a sample outlet. The sample outlet may be provided in the lower portion of the fifth chamber, for instance the lower 10%, more preferably lower 5% and ideally the lowest part of the fifth chamber. The outlet may be in the bottom wall of the chamber or preferably in a corner of the chamber, ideally the corner opposing the inlet. The sample outlet may connect to a channel. Preferably the channel has a plurality of sections. The channel may have a vertical section and/or a horizontal section and/or a second vertical section and/or second horizontal section and/or third vertical section and/or third horizontal section and/or fourth vertical section. The channel may have a vertical section and a horizontal section and a second vertical section and/or second horizontal section and/or third vertical section and/or third horizontal section and/or fourth vertical section. The channel may connect to a sixth chamber. The sixth chamber may have an inlet in the lower portion of the sixth chamber, for instance the lower 20%, preferably lower 10% and ideally lower 5%. The inlet may be in the bottom wall of the sixth chamber. The sixth chamber may have a non-linear cross-section. The sixth chamber may have a cross-section formed by a horizontal bottom wall, horizontal top wall and side walls that diverge between the bottom and the top. The corners may be provided with curved transition walls. The sixth chamber may be provided with air. The sixth chamber may have a gas outlet, for instance a vent. The gas outlet may be provided in the upper portion of the chamber, for instance the upper 20%, preferably upper 10% and ideally upper 5%. The gas outlet may be provided in the top wall of the sixth chamber. The gas outlet may lead to the outside of the device, for instance through a vent. A valve may be provided between the sixth chamber and the vent. The valve may be an open state to closed state valve. The sixth chamber may be connected to a pump, for instance an electrochemical pump. The pump may be a second pump provided on the device. The second pump may provide the drive to move one or more fluids and/or liquids through the sixth chamber and/or seventh chamber and/or into a waste chamber. The pump may provide gas to one or more of the chambers, particularly the sixth chamber. The gas may promote mixing within the one or more chambers, particularly the sixth chamber. The inlet from the pump may be provided in the upper section of the sixth chamber, for instance the upper 20%, preferably upper 5%. The channel connecting the pump to the sixth chamber may be provided with a valve. The valve may be an open state to closed state valve. The channel may include a first vertical section and/or first horizontal section and/or second vertical section and/or second horizontal section and/or third horizontal section and/or fourth vertical section. The sixth chamber may have a sample outlet. The sample outlet may be provided in the lower portion of the sixth chamber, for instance the lower 10%, more preferably lower 5% and ideally the lowest part of the sixth chamber. The outlet may be in the bottom wall of the chamber. The sample outlet may connect to a channel. Preferably the channel has a plurality of sections. The channel may have a vertical section and/or a horizontal section and/or a second vertical section and/or a second horizontal section and/or a third vertical section. The channel may have a vertical section and a horizontal section and a second vertical section and a second horizontal section and/or a third vertical section. The channel may connect to one or more further chambers, such as a seventh chamber. The sample preparation step or a part thereof may have a first state in which it is isolated from one of more of the other steps in the cartridge and/or from one or more other parts of the sample preparation step. The one or more other steps may be a sample receiving step and/or sample retention step and/or purification step and/or washing step and/or elution step and/or sample amplification step and/or electrophoresis step and/or analysis step. The sample preparation step or part thereof may have the first state during contacting of the sample with the fourth chamber and/or fifth chamber and/or sixth chamber and/or during mixing of the sample, buffer and particles and/or during heating of the sixth chamber. The sample preparation step or a part thereof may be provided with a valve. The valve may be provided at the sample outlet, preferably from the sixth chamber and/or on the channel leading from the sample preparation step to a further step and/or on the channel leading from the part of the sample preparation step to the next part of the sample preparation step and/or on the channel leading from the sample outlet. The valve may be a closed state to open state valve. The seventh chamber may have an inlet in the upper portion of the seventh chamber, for instance the upper 20%, preferably upper 10% and ideally upper 5%. The inlet may be in the top wall of the seventh chamber. The seventh chamber may have a non-linear cross-section. The seventh chamber may have a cross-section formed by a horizontal bottom wall, horizontal top wall and side walls that diverge between the bottom and the top. The corners may be provided with curved transition walls. The top wall may include a recess, such as a semi-circular recess. The seventh chamber may be provided with air. The seventh chamber may have a gas outlet, for instance a vent. The gas outlet may be provided in the upper portion of the chamber, for instance the upper 20%, preferably upper 10% and ideally upper 5%. The gas outlet may be provided in the top wall of the seventh chamber. The gas outlet may lead to the outside of the device, for instance through a vent. A valve may be provided between the seventh chamber and the vent. The valve may be an open state to closed state valve. The channel leading to the valve may connect to the seventh chamber in a recess, such as a semi-circular recess, provided in the upper section of the seventh chamber. The seventh chamber may have a second gas outlet, for instance a vent. The second gas outlet may be provided in the upper portion of the chamber, for instance the upper 20%, preferably upper 10% and ideally upper 5%. The second gas outlet may be provided in the top wall of the seventh chamber. The second gas outlet may lead to the outside of the device, for instance through a second vent. A second valve may be provided between the seventh chamber and the second vent. The second valve may be an open state to closed state valve. The second channel leading to the second valve may connect to the seventh chamber in a recess, such as a semi-circular recess, provided in the upper section of the seventh chamber. The seventh chamber may be connected to a pump, for instance an electrochemical pump. The pump may be a second pump provided on the device. The seventh chamber may be connected to the pump by a first route through the sixth chamber and/or by a second route through an eighth chamber and/or wash chamber. The sample preparation step or a part thereof may have a first state in which it is isolated from one of more of the other steps in the cartridge and/or from one or more other parts of the sample preparation step. The one or more other steps may be a sample receiving step and/or sample extraction step and/or sample retention step and/or purification step and/or washing step and/or elution step and/or sample amplification step and/or electrophoresis step and/or analysis step. The sample preparation step or part thereof may have the first state during contacting of the sample with the seventh chamber. The sample preparation step or a part thereof may be provided with one or more valves, preferably to provide the isolation. A valve may be provided at the sample outlet, preferably from the seventh chamber and/or on the channel leading from the sample preparation step to a further step and/or on the channel leading from the part of the sample preparation step to the next part of the sample preparation step and/or on the channel leading from the sample outlet. The valve may be a closed state to open state valve. One or more valves may be provided on the channel connecting the second pump to the seventh chamber by a second route. The one or more valves may include an open state to closed state valve or valves and/or a closed state to open state valve or valves. One or more valves may be provided on the channel connecting the third pump to the seventh chamber. The one or more valves may include an open state to closed state valve or valves and/or a closed state to open state valve or valves. One or more valves may be provided on the channel connecting the seventh chamber to a tenth chamber and/or waste chamber. The one or more valves may include an open state to closed state valve or valves and/or a closed state to open state valve or valves. The seventh chamber may be connected to an eighth chamber and/or wash chamber, for instance by a channel. The inlet to the eighth chamber may be provided in the upper portion of the eighth chamber, for instance the upper 20%, preferably upper 10% and ideally upper 5%. The inlet may be in the top wall of the eighth chamber. The outlet may be provided in the corner of the eighth chamber. The channel connecting the eighth chamber and/or wash chamber to the seventh chamber may have a plurality of sections. The channel may have a vertical section and/or a horizontal section and/or a second vertical section and/or a second horizontal section and/or a third vertical section and/or a third horizontal section. The channel may have a vertical section and a horizontal section and a second vertical section and a second horizontal section and/or a third vertical section and/or third horizontal section. The channel may include one or more valves. The one or more valves may include an open state to closed state valve or valves and/or a closed state to open state valve or valves. The eighth chamber and/or wash chamber may have an inlet in the upper portion of the eighth chamber, for instance the upper 20%. The eighth chamber may be connected to a pump, for instance an electrochemical pump. The pump may be a second pump provided on the device. The second pump may provide the drive to move one or more fluids and/or liquids through the eighth chamber and/or seventh chamber and/or sixth chamber and/or into a tenth chamber. The inlet from the pump may be provided in the upper section of the chamber, for instance the upper 20%, preferably upper 5%. The eighth chamber may have an outlet. The outlet may be provided in the lower portion of the chamber, for instance the lower 20%, more preferably lower 10% and ideally the lowest part of the chamber. The outlet may be in the bottom wall of the chamber. The sample preparation step or a part thereof may have a first state in which it is isolated from one of more of the other steps in the cartridge and/or from one or more other parts of the sample preparation step. The one or more other steps may be a sample receiving step and/or sample extraction step and/or purification step and/or washing step and/or elution step and/or sample amplification step and/or electrophoresis step and/or analysis step. The sample preparation step or part thereof may have the first state during contacting of the sample with the seventh chamber and/or contact between the eighth chamber and/or wash chamber and the seventh chamber. The sample preparation step or a part thereof may be provided with one or more valves, preferably to provide the isolation. The seventh chamber may be connected to a tenth chamber and/or waste chamber, for instance by a channel. The outlet from the seventh chamber may be provided in the lower portion of the seventh chamber, for instance the lower 20%, preferably lower 10% and ideally lower 5%. The outlet may be in the bottom wall of the seventh chamber. The outlet may be provided in the corner of the seventh chamber. The channel connecting the seventh chamber to the tenth chamber and/or waste chamber may have a plurality of sections. The channel may have a vertical section and/or a horizontal section and/or a second vertical section and/or a second horizontal section and/or third vertical section and/or third horizontal section and/or fourth vertical section and/or fourth vertical section and/or fifth vertical section. The channel may have a vertical section and a horizontal section and a second vertical section and a second horizontal section and third vertical section and/or third horizontal section and/or fourth vertical section and/or fourth vertical section and/or fifth vertical section. The channel may include one or more valves. The one or more valves may include a closed state to open state valve or valves, preferably provided on the channel above. The one or more valves may include an open state to closed state valve or valves, preferably provided on a parallel channel section. The parallel channel section may include a first vertical section and/or first horizontal section and/or second vertical section and/or second horizontal section and/or third vertical section and/or third horizontal section and/or fourth vertical section. The parallel channel section may be connected to the second vertical and/or third vertical sections of the channel it is provided as a parallel channel to. The tenth chamber and/or waste chamber may have a rectilinear cross-section, potentially with rounded corners. The tenth chamber and/or waste chamber may have an inlet in the upper portion of the chamber, for instance the upper 20%. The tenth chamber and/or waste chamber may be connected to a pump, for instance an electrochemical pump. The pump may be a second pump provided on the device. The second pump may provide the drive to move one or more fluids and/or liquids through the eighth chamber and/or seventh chamber and/or sixth chamber and/or into a tenth chamber. The tenth chamber and/or waste chamber may have a gas outlet, for instance a vent. The gas outlet may be provided in the upper portion of the chamber, for instance the upper 20%, preferably upper 10% and ideally upper 5%. The gas outlet may be provided in the top wall of the tenth chamber. The gas outlet may lead to the outside of the device, for instance through a vent. A valve may be provided between the tenth chamber and the vent. The valve may be an open state to closed state valve. The sample preparation step or a part thereof may have a first state in which it is isolated from one of more of the other steps in the cartridge and/or from one or more other parts of the sample preparation step. The one or more other steps may be a sample receiving step and/or sample extraction step and/or sample retention step and/or sample amplification step and/or electrophoresis step and/or analysis step. The sample preparation step or part thereof may have the first state during contacting of the sample with the seventh chamber and/or contact between the ninth chamber and/or elution chamber and the seventh chamber and/or tenth chamber and/or waste chamber. The sample preparation step or a part thereof may be provided with one or more valves, preferably to provide the isolation. The seventh chamber may be connected to the ninth chamber and/or elution chamber, for instance by a channel. The inlet to the seventh chamber may be provided in the upper portion of the seventh chamber, for instance the upper 20%, preferably upper 10% and ideally upper 5%. The inlet may be in the top wall of the seventh chamber. The inlet may be provided in the corner of the seventh chamber. The channel connecting the ninth chamber and/or elution chamber to the seventh chamber may have a plurality of sections. The channel may have a vertical section and/or a horizontal section and/or a second vertical section and/or a second horizontal section. The channel may have a vertical section and a horizontal section and a second vertical section and a second horizontal section. The channel may include one or more valves. The one or more valves may include an open state to closed state valve or valves and/or a closed state to open state valve or valves. The ninth chamber and/or elution chamber may have a circular cross-section. The cross-section may be relative to a horizontal axis. The ninth chamber and/or elution chamber may be provided with an eluent. The eluent may be provided to control conditions for a subsequent process and/or reaction, for instance in one or more further chambers and/or channels, such as the seventh chamber. The ninth chamber and/or elution chamber may have an inlet in the upper portion of the ninth chamber and/or elution chamber, for instance the upper 20%. The ninth chamber and/or elution chamber may be connected to a pump, for instance an electrochemical pump. The pump may be a third pump provided on the device. The third pump may provide the drive to move one or more fluids and/or liquids through the ninth chamber and/or elution chamber and/or seventh chamber and/or other step and/or amplification chamber. The inlet from the pump may be provided in the upper section of the chamber, for instance the upper 20%, preferably upper 5%. The ninth chamber and/or elution chamber may have an outlet. The outlet may be provided in the lower portion of the chamber, for instance the lower 20%, more preferably lower 10% and ideally the lowest part of the chamber. The outlet may be in the bottom wall of the chamber. The sample preparation step or a part thereof may have a first state in which it is isolated from one of more of the other steps in the cartridge and/or from one or more other parts of the sample preparation step. The one or more other steps may be a sample receiving step and/or sample extraction step and/or sample retention step and/or washing step and/or sample amplification step and/or electrophoresis step and/or analysis step. The sample preparation step or part thereof may have the first state during contacting of the sample with the seventh chamber and/or contact between the ninth chamber and/or elution chamber and the seventh chamber. The sample preparation step or a part thereof may be provided with one or more valves, preferably to provide the isolation. The seventh chamber may have a sample outlet. The sample outlet may be provided in the lower portion of the seventh chamber, for instance the lower 10%, more preferably lower 5% and ideally the lowest part of the seventh chamber. The outlet may be in the bottom wall of the chamber and/or in a corner of the chamber. The sample outlet may connect to a channel. Preferably the channel has a plurality of sections. The channel may have a horizontal section and/or a vertical section and/or a second horizontal section and/or a second vertical section and/or third horizontal section and/or third vertical section and/or fourth horizontal section and/or fourth vertical section and/or fifth horizontal section. The channel may have a horizontal section and a vertical section and a second horizontal section and a second vertical section and third horizontal section and/or third vertical section and/or fourth horizontal section and/or fourth vertical section and/or fifth horizontal section. The channel may connect to one or more further chambers, such as an eleventh chamber, and/or to an amplification step. The sample preparation step or a part thereof may have a first state in which it is isolated from one of more of the other steps in the cartridge and/or from one or more other parts of the sample preparation step. The one or more other steps may be a sample receiving step and/or sample retention step and/or purification step and/or washing step and/or electrophoresis step and/or analysis step. The sample preparation step or part thereof may have the first state during contacting of the sample with the seventh chamber and/or further chamber and/or amplification step. The sample preparation step or a part thereof may be provided with one or more valves. The valve may be provided at the first and/or second gas outlets for the seventh chamber and/or channel to the second pump and/or channel to the sixth chamber and/or channel to the tenth chamber.

The sample amplification step may be provided on the device. The sample amplification step may be provided on the same device as the sample receiving step and/or sample preparation step.

The sample amplification step may include a first inlet, preferably a channel. The channel may be connected to the sample receiving step and/or sample preparation step.

The sample amplification step may include a second inlet, preferably a channel. The channel may be connected to a pump, for instance the second or fourth pump on the device.

The sample amplification step may include a first outlet, preferably a channel. The channel may be connected to a sample storage step or location, for instance a chamber.

The sample amplification step may include a second outlet, for instance a channel. The channel may be connected to a further step, for instance a denaturing step and/or electrophoresis step and/or analysis step.

The first inlet and/or second outlet may be provided on the inlet channel for the amplification step. The first inlet and/or second outlet may share a section of channel and have separate channel sections. Preferably the first inlet is provided by a separate channel to either the first outlet or the second outlet.

The first outlet and/or second inlet may be provided on the outlet channel for the amplification step. The first outlet and/or second inlet may share a section of channel and have separate channel sections.

The sample amplification step may include a chamber, for instance an eleventh chamber. The chamber is preferably connected to the channel. The chamber preferably receives the sample. The sample may be a washed sample. The sample may be a purified sample. The sample may be less than the whole of the sample provided to the device.

The chamber may be provided with a curved base. The base may be semi circular in cross-section. The base may be a part of a cylinder or hemisphere or proportion thereof. The chamber may be provided with a curved top. The top may be semi circular in cross-section. The top may be a part of a cylinder or hemispherical or a portion thereof.

The top may be a larger volume than the bottom. The top hemisphere or portion thereof may be larger then the lower hemisphere or portion thereof.

A transition surface may extend between the base of the chamber and the top of the chamber.

The chamber may include a support location for one or more particles, such as a bead. The one or more particles may provide one or more or all the reagents for a reaction, particularly an amplification, such as PCR. The support location may define a position of rest for the one or more particles. Preferably in the position of rest, the one or more particles do not block or obscure an inlet to and/or outlet from the chamber. Preferably in the position of rest at least 50%, preferably at least 60% and more preferably at least 70% of the surface area of the one or more particles are exposed to the chamber.

Preferably an inlet for a sample and/or an inlet from a previous chamber is provided in a side wall of the chamber. The inlet may be provided in the mid section of the height of the chamber, preferably the middle 20%, more preferably the middle 10%.

Preferably the outlet for the sample and/or outlet to a receiving location and/or other chamber is provided in a side wall of the chamber. The outlet may be provided in the mid section of the height of the chamber, preferably the middle 20%, more preferably the middle 10%.

The inlet and the outlet are preferably provided opposite one another. The inlet and the outlet are preferably provided at the same height in the chamber.

The chamber may have an orientation of use. A chamber may be provided with a horizontal base and/or a horizontal top. The base and/or top, may be horizontal +/−10⁰, preferably +/−5⁰ and more preferably +/−3⁰.

The chamber may be provided with one or more side walls. The side wall(s) may be vertical +/−10⁰, preferably +/−5⁰ and more preferably +/−3⁰.

The chamber may include a support location for one or more particles, such as a bead. The one or more particles may provide one or more or all the reagents for a reaction, particularly an amplification, such as PCR. The support location may define a position of rest for the one or more particles. Preferably in the position of rest, the one or more particles do not block or obscure an inlet to and/or outlet from the chamber. Preferably in the position of rest at least 50%, preferably at least 60% and more preferably at least 70% of the surface area of the one or more particles are exposed to the chamber.

Preferably an inlet for a sample and/or an inlet from a previous chamber is provided in the top of the chamber or in the upper section of the chamber. The upper section may be the upper 20%, more preferably the upper 10%.

Preferably the outlet for the sample and/or outlet to a receiving location and/or other chamber is provided in the top of the chamber. The upper section may be the upper 20%, more preferably the upper 10%. The inlet and the outlet may be the same.

Preferably the chamber is provided with a chamber filling outlet. Preferably fluid enters the chamber via the inlet and flows out of the chamber through the chamber filling outlet during the filling of the chamber. The chamber filling outlet is preferably provided in the base or lower section of the chamber, for instance the lower 20% or more preferably 10%.

The channel connected to the inlet to the chamber may be provided with a valve. The channel connected to the outlet from the chamber may be provided with a valve. One or more of the valves may be open state to closed state valves, particularly for the first inlet and/or second outlet channels. One or more of the valves may be closed state to open state valves, particularly for the second inlet and/or first outlet channels. One or more of the valves may be provided closer to the chamber than the split into the first inlet and second outlet and/or second inlet and first outlet sections of channel. If the valves are provided further from the chamber than the split into the first inlet and second outlet and/or second inlet and first outlet sections of channel, then a separate valve may be provided for each channel section.

The valve connected to the inlet may provide a first sealing location. The valve connected to the outlet may provide a second sealing location. One or more interconnected channels and chambers may be provided between the first sealing location and the second sealing location. Preferably the channel connected to the inlet, the chamber and the channel connected to the outlet are provided between the first sealable location and the second sealable location.

The section of the device including the first sealable location, second sealable location and channels and chambers provided there between may have an extent, preferably in a first plane. The section of the device including the first sealable location, second sealable location and channels and chambers provided there between may have a planar form and/or planar exterior surface extending in a first plane.

A heating device may be provided to heat the chamber. The heating device may have an extent parallel to the first plane. The heating device may have an extent parallel to the first plane of the planar form of the section and/or planar exterior surface. The extent of the heating device may be greater than 75% of the extent of the channels and chambers between the first sealable location and the second sealable location. The extent of the heating device may be greater than 75% of the extent of the channels and chambers between the first sealable location and the second sealable location, considered in terms of the area those extend to in the first plane. The extent of the heating device may be greater than 80%, 90% or even 95%, possibly even 98% or 100% of such extents. The heating device may be incident with at least 75% of the extent of the channels and chambers provided between the first sealable location and the second sealable location, when the extent of those channels and chambers is projected perpendicular to the first plane. The extent of the heating device may be greater than 80%, 90% or even 95%, possibly even 98% or 100% of such extents.

The chamber may have an orientation of use. A chamber may be provided with a horizontal base and/or a horizontal top. The base and/or top, may be horizontal +/−10⁰, preferably +/−5⁰ and more preferably +/−3⁰.

The chamber may be provided with one or more side walls. The side wall(s) may be vertical +/−10⁰, preferably +/−5⁰ and more preferably +/−3⁰.

The junction between the base and the side walls may be curved. The junction between the top and the side walls may be curved. The junction between the top and the side walls may be provided by an intermediate wall. The intermediate wall may be inclined relative to the top and/or side walls.

The chamber may include a support location for one or more particles, such as a bead. The one or more particles may provide one or more or all the reagents for a reaction, particularly an amplification, such as PCR. The support location may define a position of rest for the one or more particles. Preferably in the position of rest, the one or more particles do not block or obscure an inlet to and/or outlet from the chamber. Preferably in the position of rest at least 50%, preferably at least 60% and more preferably at least 70% of the surface area of the one or more particles are exposed to the chamber. The support location may be provided by the base of the chamber.

Preferably an inlet for a sample and/or an inlet from a previous chamber is provided in the top of the chamber or in the upper section of the chamber. The upper section may be the upper 20%, more preferably the upper 10%.

The inlet may be provided in a corner of the chamber.

Preferably the outlet for the sample and/or outlet to a receiving location and/or other chamber is provided in the top of the chamber. The upper section may be the upper 20%, more preferably the upper 10%. The inlet and the outlet may be provided at the same height.

The outlet may be provided in a corner of the chamber.

An inlet channel may be provided which leads to the inlet. An outlet channel made be provided which leads away from the outlet. A by-pass channel may be provided for the chamber. The by pass channel may connect a part of the inlet channel to a part of the outlet channel.

The by-pass channel may be a continuation of the channel from which the inlet channel and/or outlet channel branch. The by-pass channel and channel may have a common axis.

The by-pass channel may be a branch from the channel from which the inlet channel branches. The by-pass channel and/or inlet channel may be provided with an axis which is not a continuation of the axis of the channel from which they branch. Preferably, the by-pass channel is provided with an axis which is not a continuation of the axis of the channel from which it branches, with still more preferably the inlet channel being provided with on a common axis to that of the portion of the channel which adjoins it.

The by-pass channel may be a branch from the channel from which the outlet channel branches. The by-pass channel and/or outlet channel may be provided with an axis which is not a continuation of the axis of the channel from which they branch. Preferably, the by-pass channel is provided with an axis which is not a continuation of the axis of the channel from which it branches, with still more preferably the outlet channel being provided with on a common axis to that of the portion of the channel which adjoins it.

Preferably one or more dimensions of the outlet channel are smaller than the corresponding dimension of the inlet channel. The value of the one or more dimensions may be considered at the location within the inlet channel and/or outlet channel where that dimension has its lowest value. The one or more dimensions may include one or more or all of the width and/or height and/or cross-sectional area. The cross-sectional area may be measured perpendicular to the direction of flow in the inlet channel and/or outlet channel and/or perpendicular to the alignment or axis of the inlet channel and/or outlet channel.

The resistance to fluid flow provided by the outlet and/or outlet channel may be greater than the resistance to fluid flow provided by the inlet and/or inlet channel. The resistance to fluid flow provided by the outlet and/or outlet channel may be greater than the resistance to fluid flow provided by the by-pass channel.

The path of least resistance for the fluid may be through the inlet and into the chamber until the fluid reaches the outlet and/or outlet channel. The path of least resistance for the fluid may be through the by-pass channel once the fluid has reached the outlet and/or outlet channel.

The fluid flow may switch from the inlet channel to the by-pass channel when a predetermined volume of fluid is provided in the chamber.

The chamber may have an orientation of use. A chamber may be provided with a curved base. The base may be semi circular. The base may be a hemisphere or proportion thereof. The chamber may be provided with a top wall, such as a planar top wall. The top wall may be provided in one or more portions. The plane of one or more of those portions may be different to the plane of one or more of the other portions. Preferably the planes are parallel.

An inclined transition surface may extend between the base of the chamber and the side walls of the chamber. The side wall may connect to the top of the chamber. The side walls may be vertical in the orientation of use.

The chamber may include a support location for one or more particles, such as a bead. The one or more particles may provide one or more or all the reagents for a reaction, particularly an amplification, such as PCR. The support location may define a position of rest for the one or more particles. Preferably in the position of rest, the one or more particles do not block or obscure an inlet to and/or outlet from the chamber. Preferably in the position of rest at least 50%, preferably at least 60% and more preferably at least 70% of the surface area of the one or more particles are exposed to the chamber.

Preferably an inlet for a sample and/or an inlet from a previous chamber is provided in a side wall of the chamber. The inlet may be provided in the lower section of the height of the chamber, preferably the lower 30%, more preferably the lower 10%.

Preferably the outlet for the sample and/or outlet to a receiving location and/or other chamber is provided in a top wall of the chamber. The outlet may be provided in the top section of the height of the chamber, preferably the top 20%, more preferably the top 10%.

The inlet and the outlet are preferably provided opposite one another. The inlet and the outlet are preferably provided at different heights in the chamber.

The sample amplification step or a part thereof may have a first state in which it is isolated from one of more of the other steps in the cartridge and/or from one or more other parts of the sample amplification step. The one or more other steps may be a sample receiving step and/or sample preparation step and/or sample retention step and/or purification step and/or washing step and/or elution step and/or further sample receiving step and/or electrophoresis step and/or analysis step. Preferably the one or more other steps may be a further sample receiving step and/or electrophoresis step and/or analysis step. The sample amplification step or part thereof may have the first state during contacting of the sample with the chamber, particularly the chamber in which amplification is provided.

The sample denaturing step may be provided on the device. The sample denaturing step may be provided on the same device as the sample receiving step and/or sample preparation step and/or further sample receiving step and/or sample amplification step. The sample denaturing step may include a chamber.

The chamber may be connected to the amplification step, preferably by a channel. The channel may be connected to the second outlet from the amplification step. The inlet to the chamber may be provided in the upper portion of the chamber, for instance the upper 20%, preferably upper 10% and ideally upper 5%. The inlet may be in the top wall of the chamber. The inlet may be provided in the corner of the chamber.

The channel connecting the amplification step and/or amplification chamber to the chamber may have a plurality of sections. The channel may have a horizontal section and/or a vertical section and/or a second horizontal section and/or a second vertical section. The channel may have a horizontal section and a vertical section and a second horizontal section and/or a second vertical section.

The channel may include one or more valves. The one or more valves may include an open state to closed state valve or valves and/or a closed state to open state valve or valves.

The chamber may have a non-linear cross-section. The chamber may have a cross-section formed by a horizontal top wall, inclined lower wall and end walls joining the top and lower walls. The transition end walls may be curved or linear. The cross-section may be relative to a horizontal axis.

The chamber may have a sample outlet. The sample outlet may be provided in the lower portion of the chamber, for instance the lower 10%, more preferably lower 5% and ideally the lowest part of the chamber. The outlet may be in the bottom wall of the chamber or preferably in a corner of the chamber, ideally the corner opposing the inlet.

The chamber may be connected to a pump, for instance an electrochemical pump. The pump may be a second or fourth pump provided on the device. The second or fourth pump may provide the drive to move one or more fluids and/or liquids through the amplification step and/or amplification chamber and/or chamber and/or one or more further chambers and/or denaturing step.

The connection to the pump may be via the amplification step and/or amplification chamber.

In one embodiment, the amplification chamber is connected to the denaturing chamber, preferably with no further chambers provided there between.

The channel leading from the amplification chamber to the denaturing chamber may split into two channels. One of the two channels may lead, preferably past a valve, to the denaturing chamber. The denaturing chamber may have a vent channel which preferably extends past a valve. One of the two channels may lead, preferably past a valve to an archive chamber. The archive chamber may have a vent channel which preferably extends past a valve.

The denaturing chamber may have an outlet channel which leads to the analysis step and/or electrophoresis step.

In a second embodiment, the pump may be connected to a channel which leads to an inlet for a further chamber. The further chamber may contain one or more reagents or materials, for instance for denaturing the sample. The further chamber may have an outlet leading to a channel and/or to the amplification step and/or to the amplification chamber.

Particularly in a second embodiment, the chamber may be connected to a second chamber, preferably by a channel. The inlet to the second chamber may be provided in the upper portion of the second chamber, for instance the upper 20%, preferably upper 10% and ideally upper 5%. The inlet may be in the top wall of the second chamber. The inlet may be provided in the corner of the second chamber.

Particularly in a second embodiment, the channel connecting the chamber to the second chamber may have a plurality of sections. The channel may have one or more horizontal sections and/or one or more vertical sections.

Particularly in a second embodiment, the second chamber may have a non-linear cross-section. The chamber may have a cross-section formed by a horizontal top wall, inclined lower wall and end walls joining the top and lower walls. The transition end walls may be curved or linear. The cross-section may be relative to a horizontal axis.

Particularly in a second embodiment, the second chamber may have a sample outlet. The sample outlet may be provided in the lower portion of the second chamber, for instance the lower 10%, more preferably lower 5% and ideally the lowest part of the second chamber. The outlet may be in the bottom wall of the second chamber or preferably in a corner of the second chamber, ideally the corner opposing the inlet.

Particularly in a second embodiment, the second chamber may be connected to a third chamber, preferably by a channel. The inlet to the third chamber may be provided in the upper portion of the third chamber, for instance the upper 20%, preferably upper 10% and ideally upper 5%. The inlet may be in the top wall of the third chamber. The inlet may be provided in the corner of the third chamber.

Particularly in a second embodiment, the channel connecting the second chamber to the third chamber may have a plurality of sections. The channel may have one or more horizontal sections and/or one or more vertical sections.

Particularly in a second embodiment, the third chamber may have a non-linear cross-section. The chamber may have a cross-section formed by a horizontal top wall, inclined lower wall and end walls joining the top and lower walls. The transition end walls may be curved or linear. The cross-section may be relative to a horizontal axis.

Particularly in a second embodiment, the third chamber may have a sample outlet. The sample outlet may be provided in the lower portion of the third chamber, for instance the lower 10%, more preferably lower 5% and ideally the lowest part of the third chamber. The outlet may be in the bottom wall of the third chamber or preferably in a corner of the third chamber, ideally the corner opposing the inlet.

The sample denaturation step or a part thereof may have a first state in which it is isolated from one of more of the other steps in the cartridge and/or from one or more other parts of the sample denaturation step. The one or more other steps may be a sample receiving step and/or sample extraction step and/or sample retention step and/or washing step and/or further sample receiving step and/or sample amplification step and/or electrophoresis step and/or analysis step. Preferably the one or more other steps may be a further sample receiving step and/or sample amplification step and/or electrophoresis step and/or analysis step. The sample denaturation step or part thereof may have the first state during contacting of the sample with the chamber and/or second chamber and/or third chamber and/or during denaturation of the sample. The sample preparation step or a part thereof may be provided with one or more valves, preferably to provide the isolation. The first inlet to the amplification step and/or amplification chamber may be provided with a valve, preferably of the open state to closed state type. The first outlet from the amplification step and/or amplification chamber may be provided with a valve, preferably of the open state to closed state type. The outlet from the denaturation step to the electrophoresis step and/or the channel connected to the outlet of the third chamber may be provided with a valve, preferably of the closed state to open state type.

The electrophoresis step may be provided on the device. The electrophoresis step may be provided on the same device as the sample receiving step and/or sample preparation step and/or further sample receiving step and/or sample amplification step. Preferably the electrophoresis step may be provided on the same device as the further sample receiving step and/or sample amplification step. The electrophoresis step may include a channel.

The channel may be connected to the amplification step and/or denaturing step. The channel may extend from the plane of the device to a location behind the plane of the device.

The electrophoresis step may be provided on an element. The element may be a part of or be separate from the device. The element may be planar. The element may include one or more channels. The element may include one or more channels in which electrophoresis is provided. One or more electrodes may be provided on the element. One or more electrodes may be provided to load the sample into the element. One or more electrodes may be provided to perform the electrophoresis step. The electrodes may be provided in portions which have a greater depth than one or more other parts of the element. The one or more other parts of the element may include the part in which the channel is provided. The electrodes may be provided in portions which adjoin end portions of the element. The end portions may provide the mounting for the element on a carrier and/or relative to the device, such as a cartridge.

The connection between the one or more electrodes and the operating electronics for the instrument may be provided by one or more pins mounted on the element. The one or more pins may be spring loaded. The one or more pins may be partially or fully recessed into a surface of the element, particularly the greater depth portion(s) thereof. The connection may be provided or may be further provided by one or more pins mounted on the instrument. The one or more pins may be spring loaded. The connection may be made when the element is put in the use position.

The element, and particularly a channel therein, may be connected to the device, such as a cartridge, by a conduit. The conduit may be flexible. The conduit may be a tube.

The channel may be connected to a chamber. The chamber may contain a liquid to matrix interface, preferably a horizontal interface. A pump, preferably an electrochemical pump, preferably the second or fourth pump may convey the sample to the chamber. The pump, preferably the electrochemical pump, may also convey a buffer and/or formamide to the chamber. The buffer and/or formamide may displace the content of the amplification and/or PCR chamber into the chamber. The buffer and/or formamide may include one or more components for the electrophoretic separation and/or analysis. The one or more components may include a size standard.

The sample may be concentrated before the start of electrophoresis.

The sample may be concentrated before the sample enters the matrix. The sample may be concentrated by the electrophoretic velocity on one side of the interface exceeding the opposing electroosmotic velocity and/or by the electrophoretic velocity on the other side of the interface being less than the opposing electroosmotic velocity.

The sample may be collecting and/or concentrated at a first location. The sample may be further collected and/or concentrated at a second location.

The sample may be collecting and/or concentrated at a first location in the form of an interface. The sample may be further collected and/or concentrated at a second location in the form of an interface. The first interface may be planar. The second interface may be planar. The first interface may be provided by a series of surfaces. The second interface may be provided by a series of surfaces. The series of surfaces may be provided by particles or beads or channels or mixtures thereof.

The sample may be collecting and/or concentrated at a first interface. The sample may be further collected and/or concentrated at a second interface. The sample may be stacked at a first interface. The sample may be further stacked at a second interface.

The first interface may be a liquid to liquid interface or liquid to solid or gel interface or solid or gel to solid or gel interface. The first interface may be a membrane. The second interface may be a liquid to liquid interface or liquid to solid or gel interface or solid or gel to solid or gel interface. The second interface may be a membrane. The first and second interfaces may be of the same or different types.

The sample or a part thereof may be collected and/or concentrated by flowing a first fluid past a first side of an interface. The sample or a part thereof may be collected and/or concentrated by flowing a second fluid past a second side of an interface.

The sample may be fed to one side of the interface, for instance the first side. A reagent, for instance a buffer may be fed to the other side of the interface, for instance the second side. The channel feeding the sample and/or the channel feeding the reagent to the channel containing the interface may be at least partially with the channel containing the interface. The channels may be aligned at an angle of less than 30°. Both channels may be so aligned. The channel feeding the sample and/or the channel feeding the reagent to the channel containing the interface may be curved so as to align their flow with the direction of flow within the channel containing the interface.

The channel for electrophoresis may be provided at an angle to the interface, for instance greater than 75°. The channel may be perpendicular to the interface, particularly the plane thereof. The channel for electrophoresis may be provided at an angle to the first and the second interface, for instance greater than 75°. The channel may be perpendicular to the first and the second interface, particularly the plane thereof.

The sample or a part thereof may be collected and/or concentrated at the first interface and then at the second interface. Conditions on one or both sides of the first interface may be varied to cause collection and/or concentration at the first interface. Conditions on one or both sides of the second interface may be varied to cause collection and/or concentration at the first interface. Conditions on one or both sides of the first interface may be varied to cause collection and/or concentration at the second interface. Conditions on one or both sides of the second interface may be varied to cause collection and/or concentration at the second interface. The conditions which are varied may be one or more of reagent or reagents present, the reagent or reagents concentration, pH, temperature, conductivity of the components present or electrical potential present. The conditions may vary at or in proximity with the interface.

The electrical potential may be applied by a voltage across a first electrode and a second electrode. The first electrode may be provided to one side of the interface, particularly the first and the second interfaces. The second electrode may be provided to the other side of the interface, particularly the first and second interfaces. The first electrode may be provided in a channel or chamber connected to, but spaced from the channel through which the sample is introduced and/or in which the interface is provided. The second electrode made be provided in the channel for electrophoresis. The second electrode may be provided beyond the channel for electrophoresis, compared with the position of the first electrode.

The second interface may lead to the channel for electrophoresis. The second interface may be in contact with the matrix within the channel for electrophoresis.

The sample may be introduced to a channel, that channel being in contact with a first interface. That channel may be in contact with a second interface. The first and second interfaces may be provided at opposing ends of the channel. The first and second interfaces may be provided in opposition to one another, with a length of channel there between. The length of channel there between may include the channel in which electrophoresis is provided.

An electrode may be provided on the side of the first interface away from the channel. An electrode may be provided on the side of the second interface away from the channel. An electrical potential may be applied to one or both of the electrodes, preferably across the electrodes.

At least a part of the sample, such as DNA in the sample, may be moved towards the first interface by the electrical potential. The first interface may be downstream of a second interface relative to the direction in which the sample flows into the channel. The electrical potential may be applied as the sample flows through the channel. The flow may be from an inlet to an outlet. A waste sample chamber may be provided downstream of the channel.

The first and/or second interface may be impermeable to one or more components of the sample, such as DNA. The first and/or second interface may be impermeable to components of greater than 5 kDa, or even greater than 8 kDa.

A further material may flow into the channel, preferably after the sample flow and/or after the at least a part of the sample is at the first interface. The further material may displace the sample flow from the channel. One or more other materials may flow through the channel between the sample flow and the further material.

The further material may provide a matrix for the electrophoresis in the channel. The further material may be introduced into the channel and then altered to provide the matrix for electrophoresis. The further material may be altered by the application of light, such as UV light, and/or heating. The further material may be altered by polymerisation.

The one or more other materials may include one or more buffers and/or one or more salt removal agents and/or one or more DNA purification reagents and/or one or more PCR primer removal reagents.

The sample may be introduced to a channel, that channel being in contact with a first interface. The first interface may be provided at one end of a channel, with a length of channel there between. The length of channel there between may include the channel in which electrophoresis is provided.

An electrode may be provided on the side of the interface away from the channel. An electrode may be provided on the other side of the interface, with the length of the channel provided between that electrode and the other electrode. An electrical potential may be applied to one or both of the electrodes, preferably across the electrodes.

At least a part of the sample, such as DNA in the sample, may be moved towards the interface by the electrical potential. The interface may be provided in a wall of the channel through which the sample flows and/or may be across a channel extending off the channel within which the sample flows. The electrical potential may be applied as the sample flows through the channel. The flow may be from an inlet to an outlet. A waste sample chamber may be provided downstream of the channel.

The first interface may be impermeable to one or more components of the sample, such as DNA. The first interface may be impermeable to components of greater than 5 kDa, or even greater than 8 kDa.

The electrical potential may be used to transfer the at least a part of the sample from the interface to the matrix in which electrophoresis is conducted. The electrical potential may be reversed to provide this transfer.

One or more other materials may flow through the channel after the sample flow. The one or more other materials may include one or more buffers and/or one or more salt removal agents and/or one or more DNA purification reagents and/or one or more PCR primer removal reagents.

The channel may be connected to the electrophoresis channel. The electrophoresis channel may be linear. The electrophoresis channel may have a side channel, preferably the side channel is connected to the channel. The electrophoresis channel may have a second side channel. The second side channel may be axially aligned with the first side channel or may be offset relative thereto. The electrophoresis channel may be provided with an electrode an one end of a separation length and a second electrode at the other end of a separation length. The first side channel and/or second side channel may be provided with an electrode. One or more of the electrodes may have a coating, for instance a platinum coating, gold coating, carbon coating, nickel coating. One or more of the electrodes may be of platinum, gold, carbon or nickel.

The channel may be provided with a first side channel through which the sample or at least a part thereof is introduced. The first side channel may provide flow in the direction of gravity to the channel. The channel may be provided with a second side channel, preferably through which the sample or a part thereof exits the channel. The second side channel ma provide flow in a direction against gravity away from the channel. The junction between the first side channel and the channel may be spaced along the channel when compared with the junction between the second side channel and the channel.

A detection location may be provided at a position along the separation length.

The sample amplification step may include a split into a first channel and a second channel. The first channel may be connected to the amplification step and/or amplification chamber as described above. The second channel may be connected to a second amplification step and/or amplification channel.

The sample amplification step may include a supply of sample to a first channel and a second channel. The first channel may be connected to the amplification step and/or amplification chamber as described above. The second channel may be connected to a second amplification step and/or amplification channel.

The first amplification step and/or amplification chamber may be connected in series with the second amplification step and/or amplification chamber. The first amplification step and/or amplification chamber may be connected in parallel with the second amplification step and/or amplification chamber.

The second amplification step and/or second amplification chamber may have any of the features, options and possibilities set out elsewhere, including those of the amplification step and/or amplification chamber.

The second amplification step and/or second amplification chamber may be provided with a quantification unit, for instance for the amount of sample therein, ideally the amount of DNA. The quantification unit may provide the amount of sample at one or more times before, during or after amplification in the second amplification step and/or second amplification chamber.

The quantification unit may include one or more reagents provided in or introduced to the second amplification step and/or second amplification chamber.

The quantification unit may include a device sensitive to a characteristic of the sample and the amount thereof. The characteristic may be light, particularly fluorescent light. The quantification unit may include the optical system and/or light detector used in the electrophoresis step and/or analysis step.

The sample preparation step may include one or more chambers, preferably into which the sample passes.

The chamber may be provided connected to one or more further chambers. Each of the chambers may be provided with a one or more particles. The particles may be beads. One or more of the particles may be magnetic. The one or more particles may have a magnetic material within a surface layer or layers. The particles may be provided with one or more reagents or materials which releasable bind and/or link and/or combine with a part of the sample, for instance DNA. The particles, such as beads, may be stored in the chamber before use. The particles, such as beads, may be introduced to the chamber to prepare it for use, for instance within 5 hours, or even within 1 hour, of use occurring.

A plurality of chamber may be provided, connected in series. Two or more of the chambers may have an inlet in the upper portion of the chamber, for instance the upper 20%. The inlet may be in the top wall of the chamber. Two or more of the chambers may have an outlet in the lower portion of the chamber, for instance the lower 20%, more preferably lower 10% and ideally the lowest part of the chamber. The outlet may be in the bottom wall of the chamber.

The chambers may be connected to each other by one or more channels. Preferably the channel has a plurality of sections. The channel may have one or more vertical sections and/or a one or more horizontal sections.

Two or more of the chambers may have the same configuration and/or shape. One or more of the chambers may have a different configuration and/or shape to one or more of the others.

One or more of the chambers may be a channel or passageway which is larger in respect of one or more dimensions than the channel leading to it and/or from it. The one or more particles may be provided in the channel or passageway which is larger.

One or more chambers may be provided having a particulate collection and/or holding location. Flow into the chamber preferably passes through the particulate collection and/or holding location, preferably preferentially to flow through other locations in the chamber. The particulate collection and/or holding location may be a recess in the bottom of the chamber.

One or more chambers, channels or passageways may be provided in which the one or more particles are provided in a channel connected to the one or more chambers, channels or passageways. The one or more particles may be displaceable from the channel into the one or more chambers, channels or passageways. A material may be provided in the channel to displace the one or more particles.

Any of the aspects of the invention may include any of the following options, features or possibilities.

The sample may be received from one or more of: a swab, a buccal swab, a cotton swab, a soft swab, a solution, a suspension, an item of clothing, an item placed in the mouth, a cigarette or piece thereof, chewing gum or saliva.

The sample may be a skin sample, blood sample, cell sample, bodily fluid sample, hair sample, saliva sample or sample containing one or more of these.

The sample may be a forensic sample. The sample may be a medical sample.

The analysis may be for diagnostic purposes. The analysis may be for forensic purposes.

The analysis may be for use in the consideration of marker targets, diagnostic assays, disease markers, biobanking applications, STR based targets in transplants, identification of drug resistant microorganisms, blood testing, mutation detection, DNA sequencing, food analysis, pharmogenetics and pharmogenomics, medical fields, biotech fields, in determining familial relationships, paternity testing and pedigree testing in animals.

The analysis may be for use in border control, security or customs situations and/or uses.

The device may be a microfluidic device. The instrument may incorporate a microfluidic device. The device may be a device processing a sample of less than 50 μl preferably less than 30 μl more preferably less than 20 μl potentially less than 10 μl in one or more steps. The device may be a device processing a fluid, particularly a liquid, of less than 50 μl preferably less than 30 μl more preferably less than 20 μl potentially less than 10 μl in one or more steps.

The device may process and/or contain a fluid, particularly a liquid, of less than 1 ml, possibly less than 500 μl possibly less than 250 μl, potentially less than 200 μl, possibly less than 175 μl, possibly less than 50 μl preferably less than 30 μl more preferably less than 20 μl potentially less than 10 μl in one or more of the following steps: a sample receiving step and/or sample preparation step and/or sample extraction step and/or sample retention step and/or purification step and/or washing step and/or elution step and/or amplification step and/or PCR step and/or denaturing step and/or investigation step and/or electrophoresis step and/or detection step and/or analysis step and/or results output step.

The device may incorporate one or more channels or chambers with a maximum dimension of less than 1000 μm, possible less than 750 μm and preferably less than 550 μm.

The device may incorporate one or more channels or chambers with a maximum dimension of less than 500 μm, possible less than 250 μm and preferably less than 100 μm.

The device may include a chambers provided with one or more reagents. One or more chambers may be so provided. The reagents may be different. The reagents may be in liquid form. The reagents may be provided on and/or in the surface of a solid. The solid may be one or more beads. The solid may be magnetic.

One or more reagents may be provided for cell lysis. One of more reagents may be provided for a selective extraction of DNA containing material from other material. One or more reagents may be provided for washing. One or more reagents may be provided for elution, particularly from the surface of a solid. One or more reagents may be provided for amplification, particularly PCR based amplification. One or more reagents may be provided for denaturing. One or more reagents may be provided for electrophoresis.

Preferably the device has a stored form and a use form. In the use form, the sample to be processed may be loaded into the device. Preferably one or more reagents are pre-loaded into the device and/or are present in the device when in the stored form. One or more reagents may be loaded into the device in the use form.

The device and/or method may include one or more pumps. Preferably the device only includes pumps of a single type. Preferably the pumps of the single type are identical with respect to chamber shape and/or electrode positions and/or electrode materials and/or orientation and/or chamber volume and/or pump electrolyte and/or pump electrolyte concentration.

One or more, preferably all, of the pumps may be electrochemical pumps.

The device may have an orientation of use, preferably one electrode in the pump chamber is provided above the other. The pump chamber may have a height greater than its width. The pump chamber may have a width greater than its depth.

The pump chamber may have an outlet. Preferably the outlet is provided in the upper section of the pump chamber. The upper section may be the upper 20%, preferably 10%, and more preferably 5% of the height of the chamber. The outlet may be in the top wall of the chamber.

The pump chamber may contain NaCl. The molarity of the electrolyte in the pump chamber may be between 0.2M and 3M, preferably 1M+/−15%.

The electrophoresis step and/or electrophoresis cartridge section may be provided with a channel, for instance a capillary for electrophoresis.

The channel may be provided with a matrix. Preferably the matrix resists the passage of elements, the resistance being related to the size of the element. Preferably different size elements migrate through the matrix at different rates, the larger migrating slower.

The channel may be provided with an inert bed of particulate material to form the matrix.

The channel may be provided with a gel, particularly a polymer gel. The channel may be provided with polyhydroacrylamide, polydimethylacrylamide or mixtures there of. The channel may be provided with a cross-linked polymer. The cross-linking of the polymer may be provided in situ.

One or more surfaces of the channel may be treated, for instance with a hydrophilic coating, for instance poly(hydroxyethlacrylamide).

The channel may be provided with a matrix during electrophoresis. The channel may be provided without a matrix prior to electrophoresis, with the matrix being introduced before electrophoresis commences. The matrix or a material for forming the matrix may be stored at a location removed from the channel in which electrophoresis is provided. The matrix or material for forming the matrix may be stored in a chamber. The chamber may be connected by a channel to the channel in which electrophoresis is provided.

The matrix and/or material for forming the matrix may be altered before use in the electrophoresis step. The alteration may be provided before and/or during and/or after the matrix and/or material for forming the matrix is provided in the channel. The alteration may be polymerisation. The alteration may be caused and/or triggered by heating and/or the application of light, such as U/V light. The alteration may be applied to all of the matrix and/or material for forming the matrix or only a part thereof. One or more parts of the matrix may be prevented from alteration, for instance by masking those parts and/or excluding heat and/or excluding light from them.

Preferably the further sample receiving step may include the transfer of a sample from outside the device and/or instrument, to inside the device and/or instrument. The sample receiving step may include the transfer of a sample from outside the device and/or instrument, to inside the device and/or instrument. Preferably the further sample receiving step may receive the sample from a collection device or from a storage device. The sample receiving step may receive the sample from a collection device or from a storage device. Preferably the sample receiving step may include the transfer of the sample to a channel or chamber within the device. The sample receiving step may include the transfer of the sample to a channel or chamber within the device.

Particularly in embodiments where one or more of a sample receiving step and/or sample preparation step and/or sample extraction step and/or sample retention step and/or purification step and/or washing step and/or elution step are provided by the instrument and/or device, then the following features may individually and/or in combinations be provided.

The sample preparation step may include contacting the sample with one or more reagents and/or one or more other components. The reagents and/or other component may be used to prepare the sample for one or more of the subsequent steps. The sample extraction step may be part of or separate from the sample preparation step. The sample extraction step may include contacting the sample with one or more reagents and/or components which select the sample component(s) relative to one or more waste components in the sample. The selected sample component(s) may be removed from the waste component(s) and/or the waste component(s) may be removed from the selected sample components. The waste component(s) may flow away from the extraction step. The waste component(s) may be washed away from the extraction step using one or more further reagents and/or components. The sample retention step may be a part of or may be separate from the sample preparation step and/or sample extraction step. The sample retention step may include contacting the sample with one or more reagents and/or components which retain the sample component(s) relative to one or more waste components in the sample. The sample component(s) may be retained on one or more beads. The beads may be magnetic. The retained sample component(s) may be removed from the waste component(s) and/or the waste component(s) may be removed from the retained sample components. The waste component(s) may flow away from the retention step. The waste component(s) may be washed away from the retention step using one or more further reagents and/or components. The waste component(s) may flow past the location of retention. The waste component(s) may be washed away using one or more further reagents and/or components which flow past the location of retention. The retained and/or selected sample may be eluted, preferably with the eluent conveying the retained and/or selected sample to the next step. The purification step may be a part of or may be separate from the sample preparation step and/or sample extraction step and/or sample retention step. The purification step may separate the selected sample components, for instance DNA, from one or more waste components of the sample, for instance cellular material, PCR inhibitors and chemical inhibitors. The washing step may be a part of or may be separate from the sample preparation step and/or sample extraction step and/or sample retention step and/or purification step. The washing step may remove one or more components of the sample from the location of one or more other components of the sample. The elution step may be a part of or may be separate from the sample preparation step and/or sample extraction step and/or sample retention step and/or purification step and/or washing step. The elution step may remove one or more components of the sample from a first form into a second form. The first form may be bound to a surface or substrate, for instance on a bead. The second form may be in a liquid, for instance the eluent.

The amplification step may include contacting the sample with one or more reagents and/or components to cause amplification. The amplification step may include contacting the sample with conditions, preferably of a cyclic nature, to cause amplification. The amplification may be provided by a PCR step.

The denaturing step may prepare the sample for electrophoresis. The denaturing step may include contacting the sample with one or more reagents and/or components. The denaturing step may include contacting the sample with conditions, preferably of a cyclic nature, to cause denaturing.

The investigation step may provide a characteristic for one component of the sample which differs from the characteristic for one or more other components of the sample. The characteristic may be one or more detectable positions and/or one or more signals and/or one or more intensities and/or one or more colours and/or one or more concentrations and/or presence of one or more characteristics and/or absence of one or more characteristics.

The electrophoresis step may be part of or may be separate from the investigation step. The electrophoresis step may include transferring the sample to a start location for electrophoresis and/or a mobility based separation and/or a size based separation. The start location may be in a channel. The electrophoresis step may include one or more voltage conditions. One or more voltage conditions may be used to transfer the sample to the start location. One or more voltage conditions may be used to provide the separation.

The analysis step may establish one or more of the characteristics of the sample. The analysis may interrogate the instrument, particularly the device, and/or may seek a response from the instrument, particularly the device. The analysis may subject the instrument, particularly the device, to an operation, for instance the application of light. The analysis may consider the response to the operation, for instance the light returning.

The analysis step may include one or more operations involving an interaction with the device. The analysis step may include one or more operations not involving an interaction with the device. One or more of the interactions may be electromagnetic interactions.

The analysis step may apply light to the device. The analysis step may receive light from the device. The analysis step may establish the relative position of the elements having a characteristic, for instance an allele having a fluorescent dye. The analysis step may establish the relative size of the elements having a characteristic, for instance an allele having a fluorescent dye. The analysis step may generate one or more results. The light may be of visible and/or non-visible wavelengths.

The results output step may display the one or more results from the analysis step and/or a processed form thereof.

The results output step may transmit the one or more results from the analysis step and/or a processed form thereof to a remote location. The results output step may compile the one or more results into a transmission form. The transmission may be via a telecommunications network. The results may be provided in a format compatible with one or more software applications, for instance one or more software applications for

The results output step may be followed by a further processing step. The further processing may interpret the results to provide further results. The further processing step may analyse the results to provide a DNA profile for the sample. The further processing step may provide an indication of a match between the sample and a database record of a sample. The further processing step may be provided at a location remote from the instrument. The further processing step may be provided at a location connected to the instrument, at least part of the time, by a telecommunications network. The further processing step may return to the instrument and/or a computer, preferably within 200 m of the site of the instrument, the further processed results.

The results may be processed on the instrument to give processed results. The processed results may extract from the results the signals, sections of signals or positions attributable to a characteristic being analysed for, such as an allele. The results and/or processed results may be provided to the results output step.

According to a fourth aspect of the invention, there is provided a method of providing a storable sample, the method including:
a) introducing a sample to a device; and
b) conveying at least a part of the sample to a receiving location to provide a storable sample.

The method may include conveying a liquid to the receiving location. The method may include conveying a DNA containing material to the receiving location.

The method may provide a storable sample of a sample on which one or more processes and/or reactions are performed. The storable sample may be provided before one or more processes and/or reactions are performed. The storable sample may be provided after one or more processed and/or reactions are performed.

The method may provide a storable sample which is one part of the sample. One or more other parts of the sample may be used in one or more processes and/or reactions. The sample may have one or more processes and/or reactions performed on it prior to taking the one part of the sample to provide the storable sample.

The one or more processes and/or one or more reactions may include one or more of: cell lysis, mixing, a surface based reaction, washing, elution, selective separation of DNA from one or more other materials, application of a magnetic field, removal of a magnetic field and one or more repeats thereof. The one or more processes and/or one or more reactions may include one or more of amplification, PCR, detection and denaturation.

The at least a part of the sample may be conveyed using one or more components of the device. The components may include one or more channels and/or chambers and/or valves.

The method may include one or more of the following steps:

passing the sample through one or more channels and/or chambers to mix the sample with one or more fluids and/or solids;

increasing the temperature of the sample and/or a mixture including the sample, preferably whilst in a chamber;

holding the sample and/or a mixture including the sample in a chamber for a period of time;

passing the sample through one or more further channels and/or further chambers;

retaining at least a part of the sample in a chamber, preferably on a surface of one or more solids, preferably using a magnetic field;

washing at least another part of the sample from the chamber where the at least a part of the sample is retained;

eluting the retained part of the sample into a fluid.

The method may include transferring at least a part of the sample from a reaction chamber to the receiving location. The method may include passing the storable sample through the reaction chamber and then on to the receiving location. The method may include passing the storable sample through an inlet into the reaction chamber and out through a separate outlet from the reaction chamber. The reaction chamber may be a PCR reaction chamber. Preferably the at least a part of the sample may be transferred prior to performing a reaction in the reaction chamber. The at least a part of the sample may be transferred during the performance of a reaction in the reaction chamber. The at least a part of the sample may be transferred after performing a reaction in the reaction chamber.

The method may provide sample to a reaction chamber, with part of the sample progressing to the receiving location when the amount of sample in the reaction chamber exceeds a predetermined amount.

The method may include transferring at least a part of the sample to the receiving location before that part of the sample reaches a reaction chamber, particularly a PCR reaction chamber. The method may include providing a split in a channel and/or chamber to feed part of the sample to a reaction chamber and part of the sample to the receiving location, preferably without entering the reaction chamber. Preferably the at least a part of the sample may be transferred prior to performing a reaction in the reaction chamber. The at least a part of the sample may be transferred during the performance of a reaction in the reaction chamber. The at least a part of the sample may be transferred after performing a reaction in the reaction chamber.

The method may provide sample to the reaction chamber, with part of the sample progressing to the receiving location when the reaction chamber is full of sample.

The at least a part of the sample transferred to the receiving location may be surplus sample.

The receiving location may be provided in a section of the device that may be detached from the device. The method may include detaching the receiving location and/or section from the device, preferably after the storable sample has been provided to the receiving location.

The method may include detaching the receiving location and/or section from the device by snapping the material joining the two. The method may include detaching the receiving location and/or section from the device by breaking the material joining the two, for instance along a line of weakness.

The method may include sealing the channel leading to the receiving location. The method may include sealing a channel and/or vent leading from the receiving location. One or both of the seals are preferably provided on the section after the section is detached from the device.

The method may include sealing the channel on the device side of the location where the channel is detached when the section is detached from the device.

The method may seal the channel between the reaction chamber and the receiving location in a horizontal section and/or a vertical section and/or diagonal section. Preferably the method seals the channel, at one or more of the locations, on a horizontal section, and ideally between one or two vertical sections.

According to a fifth aspect of the invention there is provided a device, the device having:
a) an entry location;
b) a channel connected to the entry location;
c) a receiving location, the receiving location being connected to the channel.

The receiving location may be a container for a liquid. The receiving location may be a container for a DNA containing material.

The receiving location may be an integral part of the device.

The receiving location may be detachable from the device. The receiving location may be provided in a section of the device. Preferably the section is detachable from the device. An area or line of weakness may be provided between the device and the receiving location and/or section. The section may be connected to the device at a line of weakness.

The device may be provided with a first valve in the section, preferably for sealing the channel between the receiving location and the part of the channel disrupted when the section is detached from the device. The device may be provided with a second valve not on the section, preferably for sealing the channel between the part of the channel disrupted when the section is detached from the device and the remainder of the device. The device may be provided with a third valve in the section, preferably for sealing the vent of the receiving location and/or a channel leading from the receiving location to the vent of the receiving location.

The receiving location may be a chamber. The receiving location may have an inlet in the top of the receiving location, the device having an orientation of use. The receiving location may have an outlet in the top of the receiving location, the device having an orientation of use.

The section may extend from the device. The section may be provided on one side of the device.

The maximum dimension of the section may be less than 20% the maximum dimension of the device, preferably less than 10%, more preferably less than 7.5% and ideally less than 5%. The maximum dimension of the section may be the width of the section.

The device may have an orientation of use, the maximum height of the section may be less than 20% the maximum height of the device, preferably less than 10%, more preferably less than 7.5% and ideally less than 5%.

The device may have an orientation of use, the maximum width of the section may be less than 20% the maximum dimension of the device, preferably less than 10%, more preferably less than 7.5% and ideally less than 5%.

The device may have an orientation of use, the depth of the section may be the same as the remainder of the device.

The volume of the section may be less than 5% of the volume of the device excluding the section, more preferably less than 3% and ideally less than 1%.

The device may have an orientation of use, the channel between the device and the receiving location may include a horizontal section and/or a vertical section. Preferably the sealing of the channel, at one or more of the locations, is provided on a horizontal section, and ideally between one or two vertical sections.

The section may be provided with an identifier, such as a barcode. The identifier may be the same identifier information or include the same identifier information as an identifier provided on the remainder of the device.

The device may provide one or more processing locations and/or reaction locations between the entry location and the receiving location. The device may provide one or more processing locations and/or reaction locations between the entry location and an output location. The device may provide a splitting location. The device may provide a splitting location from which a channel extends to the receiving location and/or a separate channel extends to the output location. The channels may extend directly or via one or more intermediate chambers, locations or other channels. Preferably no processing locations and/or reaction locations are provided between the splitting location and the receiving location. The device may provide one or more processing locations and/or reaction locations between the splitting location and the output location.

The one or more processing locations may be channels and/or chambers. The processing locations may include one or more of: a mixing location, a washing location, a selective separation location for DNA from one or more other material, an amplification process location, a location at which a magnetic field is applied and/or removed and/or varied and one or more repeats of these.

The one or more reaction locations may be channels and/or chambers. The reaction locations may be one or more of: a cell lysis location, a surface based reaction location, a selective separation location of DNA from one or more other materials, an amplification reaction location, a location at which a magnetic field is applied and/or removed and/or varied and one or more repeats of these.

The device may include one or more chambers. The device may include one or more channels. The device may include one or more valves. The device may include one or more vents. The device may include one or more pumps, particularly electrochemical pumps.

The device may include a reaction chamber connected to the receiving location. The device may include an inlet to a reaction chamber and an outlet from the reaction chamber to the receiving location. The reaction chamber may be a PCR reaction chamber.

The device may have an orientation of use, the device potentially including a reaction chamber, with an outlet positioned at a predetermined height in the reaction chamber. The reaction chamber may have a predetermined volume below the height of the outlet.

The splitting location may be provided with one channel connecting to the receiving location and another channel connecting to a reaction chamber, particularly a PCR reaction chamber.

According to a sixth aspect of the invention we provide a method of producing a device, the method including:
a) forming an entry location in one or more components of the device;
b) forming a channel in one or more components of the device;
c) providing a receiving location in one or more components of the device;
d) assembling the one or more components to a device;
wherein the entry location is connected to the channel and the channel is connected to the receiving location.

The fourth and/or fifth and/or sixth aspects of the invention may include any of the features, options or possibilities set out elsewhere in this application, including in the other aspects of the invention, the specific description of the embodiments and the drawings.

According to a seventh aspect, the invention provides a device, the device including one or more chambers.

The chamber may have an orientation of use. A chamber may be provided with an inclined base. The base may be inclined at 20⁰±/−10⁰, preferably +/−5⁰ and more preferably +/−3⁰.

The chamber may have a vertical side wall. Preferably the chamber has two side walls and both are vertical side walls. The side wall or side walls may be curved.

Preferably an inlet for a fluid and/or an inlet from a previous chamber is provided in the top wall of the chamber or in the top section of the side wall of the chamber. The top section may be the upper 20% of the height of the chamber, more preferably upper 10%. Preferably the inlet is provided in an upper corner of the chamber.

Preferably the outlet for the chamber is provided in the bottom wall of the chamber or in the bottom section of the side wall of the chamber. The bottom section may be the lower 10% of the height of the chamber, more preferably lower 5%. Preferably the outlet is provided in a lower corner of the chamber.

Preferably the inlet and the outlet are provided in opposing corners of the chamber.

The chamber may provide a flow path for a liquid entering the chamber, that flow path being non-laminar. Preferably the flow path extends from the inlet down the inclined base of the chamber to an outlet.

The top wall, excluding any recesses present, may be may be horizontal +/−10⁰, preferably +/−5⁰ and more preferably +/−3⁰.

Two or more such chambers may be provided in series.

The chamber may have an orientation of use. A chamber may be provided with a horizontal base. The base may be horizontal +/−10⁰, preferably +/−5⁰ and more preferably +/−3⁰.

The chamber may have an inclined side wall. Preferably the chamber has two side walls and both are inclined side walls. The side wall(s) may be inclined at between 50⁰ and 85⁰ to the horizontal, preferably between 65⁰ and 80⁰. The second side wall is preferably inclined in the opposite direction to the first side wall. The first and second side wall may be inclined at the same angle.

Preferably an inlet for a displacing fluid and/or an inlet from a pump is provided in the top wall of the chamber or in the top section of the side wall of the chamber. The top section may be the upper 20% of the height of the chamber, more preferably upper 10%. Preferably the inlet is provided in an upper corner of the chamber.

Preferably the outlet for the chamber is provided in the bottom wall of the chamber or in the bottom section of the side wall of the chamber. The bottom section may be the lower 10% of the height of the chamber, more preferably lower 5%. Preferably the outlet is provided in a lower corner of the chamber.

Preferably the inlet and the outlet are provided in corners of the chamber on the same side of the chamber.

Preferably an inlet for a sample and/or an inlet from a sample containing chamber is provided in the bottom wall of the chamber or in the bottom section of the side wall of the chamber. The bottom section may be the lower 10% of the height of the chamber, more preferably lower 5%. Preferably the inlet is provided away from the corners of the base of the chamber.

The chamber may be provided with one or more vents. One or more of the vents may be provided with one or more valves, preferably valves moving from an open state to a closed state. One or more vents may be provided in the upper section of the chamber. The upper section may be the upper 20% of the height of the chamber, more preferably upper 10%. One or more vents may connect to the chamber at a position higher than the inlet for a displacing and/or an inlet from a pump. The top wall, excluding any recesses present, may be may be horizontal +/−10⁰, preferably +/−5⁰ and more preferably +/−3⁰.

The chamber may have an orientation of use. A chamber may be provided with a horizontal base. The horizontal base may provide a retention location for one or more particles in the chamber. The one or more particles may be drawn to the retention location by a magnetic field. The highest strength magnetic field within the chamber is preferably provided at the base. The base may be horizontal +/−10⁰, preferably +/−5⁰ and more preferably +/−3⁰.

The chamber may have an inclined side wall. Preferably the chamber has two side walls and both are inclined side walls. The side wall(s) may be inclined at between 20⁰ and 80⁰ to the horizontal, preferably between 30⁰ and 60⁰. The second side wall is preferably inclined in the opposite direction to the first side wall. The first and second side wall may be inclined at the same angle.

Preferably an inlet for a wash and/or an inlet from a wash storage chamber is provided in the top wall of the chamber or in the top section of the side wall of the chamber. The top section may be the upper 20% of the height of the chamber, more preferably upper 10%. Preferably the inlet is provided in an upper corner of the chamber.

Preferably the outlet for the wash and/or outlet to a waste storage chamber is provided in the bottom wall of the chamber or in the bottom section of the side wall of the chamber. The bottom section may be the lower 10% of the height of the chamber, more preferably lower 5%. Preferably the outlet is provided in a lower corner of the chamber.

Preferably the inlet and the outlet are provided in opposing corners of the chamber.

The chamber may provide a flow path for a liquid entering the chamber, that liquid being denser than the liquid in the chamber before. Preferably the flow path extends from the inlet down an inclined side wall of chamber and/or across the bottom of the chamber to an outlet. Preferably the flow path passes through the region of the chamber with the highest magnetic field strength.

Preferably an inlet for an eluent and/or an inlet from an eluent storage chamber is provided in the top wall of the chamber or in the top section of the side wall of the chamber. The top section may be the upper 20% of the height of the chamber, more preferably upper 10%. Preferably the inlet is provided in an upper corner of the chamber.

Preferably the outlet for the eluent and/or outlet to a further chamber, preferably a PCR reaction chamber, is provided in the bottom wall of the chamber or in the bottom section of the side wall of the chamber. The bottom section may be the lower 10% of the height of the chamber, more preferably lower 5%. Preferably the outlet is provided in a lower corner of the chamber.

Preferably the inlet and the outlet are provided in opposing corners of the chamber.

The chamber may provide a flow path for a liquid entering the chamber, that liquid being denser than the liquid in the chamber before. Preferably the flow path extends from the inlet down an inclined side wall of chamber and/or across the bottom of the chamber to an outlet. Preferably the flow path passes through the region of the chamber with the highest magnetic field strength.

Preferably the inlet for the wash and/or inlet from a wash storage chamber is provided in one, preferably upper, corner of the chamber and an inlet for an eluent and/or an inlet from an eluent storage chamber is provided in another, preferably upper, corner of the chamber. Preferably the outlet for the wash and/or outlet to a waste storage chamber is provided in one, preferably lower, corner of the chamber and an outlet for the eluent and/or outlet to a further chamber is provided in another, preferably lower, corner of the chamber.

The chamber may be provided with one or more vents. One or more of the vents may be provided with one or more valves, preferably valves moving from an open state to a closed state. One or more vents may be provided in the upper section of the chamber. The upper section may be the upper 20% of the height of the chamber, more preferably upper 10%. One or more vents may connect to the chamber at a position higher than the inlet for a wash and/or an inlet from a wash storage chamber and/or the inlet for an eluent and/or an inlet from an eluent storage chamber. One or more vents may be provided in a recess extending above the top wall of the chamber. The recess may be semi-circular. The top wall, excluding the recess if present, may be may be horizontal +/−10⁰, preferably +/−5⁰ and more preferably +/−3⁰.

The chamber may have an orientation of use. A chamber may be provided with a curved base. The base may be semi circular. The base may be a hemisphere or proportion thereof. The chamber may be provided with a curved top. The top may be semi circular. The top may be hemispherical or a portion thereof.

The top may be a larger volume than the bottom. The top hemisphere or portion thereof may be larger then the lower hemisphere or portion thereof.

A transition surface may extend between the base of the chamber and the top of the chamber.

The chamber may include a support location for one or more particles, such as a bead. The one or more particles may provide one or more or all the reagents for a reaction, particularly an amplification, such as PCR. The support location may define a position of rest for the one or more particles. Preferably in the position of rest, the one or more particles do not block or obscure an inlet to and/or outlet from the chamber. Preferably in the position of rest at least 50%, preferably at least 60% and more preferably at least 70% of the surface area of the one or more particles are exposed to the chamber.

Preferably an inlet for a sample and/or an inlet from a previous chamber is provided in a side wall of the chamber. The inlet may be provided in the mid section of the height of the chamber, preferably the middle 20%, more preferably the middle 10%.

Preferably the outlet for the sample and/or outlet to a receiving location and/or other chamber is provided in a side wall of the chamber. The outlet may be provided in the mid section of the height of the chamber, preferably the middle 20%, more preferably the middle 10%.

The inlet and the outlet are preferably provided opposite one another. The inlet and the outlet are preferably provided at the same height in the chamber.

The chamber may have an orientation of use. A chamber may be provided with a horizontal base and/or a horizontal top. The base and/or top, may be horizontal +/−10⁰, preferably +/−5⁰ and more preferably +/−3⁰.

The chamber may be provided with one or more side walls. The side wall(s) may be vertical +/−10⁰, preferably +/−5⁰ and more preferably +/−3⁰.

The chamber may include a support location for one or more particles, such as a bead. The one or more particles may provide one or more or all the reagents for a reaction, particularly an amplification, such as PCR. The support location may define a position of rest for the one or more particles. Preferably in the position of rest, the one or more particles do not block or obscure an inlet to and/or outlet from the chamber. Preferably in the position of rest at least 50%, preferably at least 60% and more preferably at least 70% of the surface area of the one or more particles are exposed to the chamber.

Preferably an inlet for a sample and/or an inlet from a previous chamber is provided in the top of the chamber or in the upper section of the chamber. The upper section may be the upper 20%, more preferably the upper 10%.

Preferably the outlet for the sample and/or outlet to a receiving location and/or other chamber is provided in the top of the chamber. The upper section may be the upper 20%, more preferably the upper 10%. The inlet and the outlet may be the same.

Preferably the chamber is provided with a chamber filling outlet. Preferably fluid enters the chamber via the inlet and flows out of the chamber through the chamber filling outlet during the filling of the chamber. The chamber filling outlet is preferably provided in the base or lower section of the chamber, for instance the lower 20% or more preferably 10%.

The chamber may have an orientation of use. A chamber may be provided with a horizontal base and/or a horizontal top. The base and/or top, may be horizontal +/−10⁰, preferably +/−5⁰ and more preferably +/−3⁰.

The chamber may be provided with one or more side walls. The side wall(s) may be vertical +/−10⁰, preferably +/−5⁰ and more preferably +/−3⁰.

The junction between the base and the side walls may be curved. The junction between the top and the side walls may be curved. The junction between the top and the side walls may be provided by an intermediate wall. The intermediate wall may be inclined relative to the top and/or side walls.

The chamber may include a support location for one or more particles, such as a bead. The one or more particles may provide one or more or all the reagents for a reaction, particularly an amplification, such as PCR. The support location may define a position of rest for the one or more particles. Preferably in the position of rest, the one or more particles do not block or obscure an inlet to and/or outlet from the chamber. Preferably in the position of rest at least 50%, preferably at least 60% and more preferably at least 70% of the surface area of the one or more particles are exposed to the chamber. The support location may be provided by the base of the chamber.

Preferably an inlet for a sample and/or an inlet from a previous chamber is provided in the top of the chamber or in the upper section of the chamber. The upper section may be the upper 20%, more preferably the upper 10%.

The inlet may be provided in a corner of the chamber.

Preferably the outlet for the sample and/or outlet to a receiving location and/or other chamber is provided in the top of the chamber. The upper section may be the upper 20%, more preferably the upper 10%. The inlet and the outlet may be provided at the same height.

The outlet may be provided in a corner of the chamber.

An inlet channel may be provided which leads to the inlet. An outlet channel made be provided which leads away from the outlet. A by-pass channel may be provided for the chamber. The by pass channel may connect a part of the inlet channel to a part of the outlet channel.

The by-pass channel may be a continuation of the channel from which the inlet channel and/or outlet channel branch. The by-pass channel and channel may have a common axis.

The by-pass channel may be a branch from the channel from which the inlet channel branches. The by-pass channel and/or inlet channel may be provided with an axis which is not a continuation of the axis of the channel from which they branch. Preferably, the by-pass channel is provided with an axis which is not a continuation of the axis of the channel from which it branches, with still more preferably the inlet channel being provided with on a common axis to that of the portion of the channel which adjoins it.

The by-pass channel may be a branch from the channel from which the outlet channel branches. The by-pass channel and/or outlet channel may be provided with an axis which is not a continuation of the axis of the channel from which they branch. Preferably, the by-pass channel is provided with an axis which is not a continuation of the axis of the channel from which it branches, with still more preferably the outlet channel being provided with on a common axis to that of the portion of the channel which adjoins it.

Preferably one or more dimensions of the outlet channel are smaller than the corresponding dimension of the inlet channel. The value of the one or more dimensions may be considered at the location within the inlet channel and/or outlet channel where that dimension has its lowest value. The one or more dimensions may include one or more or all of the width and/or height and/or cross-sectional area. The cross-sectional area may be measured perpendicular to the direction of flow in the inlet channel and/or outlet channel and/or perpendicular to the alignment or axis of the inlet channel and/or outlet channel.

The resistance to fluid flow provided by the outlet and/or outlet channel may be greater than the resistance to fluid flow provided by the inlet and/or inlet channel. The resistance to fluid flow provided by the outlet and/or outlet channel may be greater than the resistance to fluid flow provided by the by-pass channel.

The path of least resistance for the fluid may be through the inlet and into the chamber until the fluid reaches the outlet and/or outlet channel. The path of least resistance for the fluid may be through the by-pass channel once the fluid has reached the outlet and/or outlet channel.

The fluid flow may switch from the inlet channel to the by-pass channel when a predetermined volume of fluid is provided in the chamber.

The chamber may have an orientation of use. A chamber may be provided with a curved base. The base may be semi circular. The base may be a hemisphere or proportion thereof. The chamber may be provided with a top wall, such as a planar top wall. The top wall may be provided in one or more portions. The plane of one or more of those portions may be different to the plane of one or more of the other portions. Preferably the planes are parallel.

An inclined transition surface may extend between the base of the chamber and the side walls of the chamber. The side wall may connect to the top of the chamber. The side walls may be vertical in the orientation of use.

The chamber may include a support location for one or more particles, such as a bead. The one or more particles may provide one or more or all the reagents for a reaction, particularly an amplification, such as PCR. The support location may define a position of rest for the one or more particles. Preferably in the position of rest, the one or more particles do not block or obscure an inlet to and/or outlet from the chamber. Preferably in the position of rest at least 50%, preferably at least 60% and more preferably at least 70% of the surface area of the one or more particles are exposed to the chamber.

Preferably an inlet for a sample and/or an inlet from a previous chamber is provided in a side wall of the chamber. The inlet may be provided in the lower section of the height of the chamber, preferably the lower 30%, more preferably the lower 10%.

Preferably the outlet for the sample and/or outlet to a receiving location and/or other chamber is provided in a top wall of the chamber. The outlet may be provided in the top section of the height of the chamber, preferably the top 20%, more preferably the top 10%.

The inlet and the outlet are preferably provided opposite one another. The inlet and the outlet are preferably provided at different heights in the chamber.

The chamber may at least in part be defined by a rotatable element. The rotatable element may provide one or more walls of the chamber. The rotatable element may provide the front, back and side wall of the chamber. The chamber may be a cylinder or section thereof. The rotatable element may provide one or more of the front and back walls of the chamber, with the device providing the other walls not provided by the chamber. One or more through apertures may be provided in a wall or walls of the chamber. The front and/or back walls may be planar.

One or more parts may be provided on the rotatable element and/or device to limit rotation of the rotatable element, for instance at the first and/or second and/or third positions.

The rotatable element may be a snug fit within a recess in the device, such as a cartridge. One or more contacts between the rotatable element and the device may be provided with a seal and/or sealing material.

The chamber may be rotated by engaging an actuator with the chamber, for instance with the front or rear wall thereof.

The rotatable element may have a first position and a second position. In the first position one or more channels may be in fluid communication with the inside of the chamber. In the first position one or more channels may not be in fluid communication with the inside of the chamber. In the second position one or more different channels may be in fluid communication with the inside of the chamber. In the second position one or more different channels may not be in fluid communication with the inside of the chamber.

In the first position an inlet channel may be in fluid communication with the inside of the chamber. In the first position an outlet channel, such as a venting channel, may be in fluid communication with the inside of the chamber. In the first position a further outlet channel, such as a discharge outlet channel, may not be in fluid communication with the inside of the chamber.

In the second position an inlet channel may not be in fluid communication with the inside of the chamber. In the second position an outlet channel, such as a venting channel, may be in fluid communication with the inside of the chamber. In the second position a further outlet channel, such as a discharge outlet channel, may be in fluid communication with the inside of the chamber.

In the first position an inlet channel may be in fluid communication with the inside of the chamber. In the first position an outlet channel, such as a venting channel, may be in fluid communication with the inside of the chamber. In the first position a further inlet channel may not be in fluid communication with the inside of the chamber. In the first position a further outlet channel, such as a discharge outlet channel, may not be in fluid communication with the inside of the chamber.

In the second position an inlet channel may not be in fluid communication with the inside of the chamber. In the second position an outlet channel, such as a venting channel, may not be in fluid communication with the inside of the chamber. In the second position a further inlet channel may be in fluid communication with the inside of the chamber. In the second position a further outlet channel, such as a discharge outlet channel, may be in fluid communication with the inside of the chamber.

A third position may be provided. The third position may be intermediate the first and second positions. In the third position the combination of channels in fluid communication with the chamber and/or not in fluid communication with the chamber may be different than in the first and/or second position. The third position may provide that no channels are in fluid communication with the inside of the chamber. One or more steps or processes may be applied to the contents of the chamber when in the third position. The one or more steps or processes may include an amplification step and/or PCR step or one or more sub-steps thereof.

One or more of the channels may be used to inspect the contents of the chamber, for instance by introducing light and/or considering the light returning from the chamber.

According to an eighth aspect, the invention provides a method of controlling the passage of one or more materials within a device, the method including:

moving one or more materials from a channel into a chamber connected to the channel;

moving one or more of the materials from the chamber into a channel connected to the chamber.

According to a ninth aspect, the invention provides a method of producing a device, the method including:

forming a recess in one or more components of the device;

forming a channel in one or more components of the device;

assembling the one or more components to form a chamber from the recess;

wherein the chamber is connected to the channel.

The seventh and/or eighth and/or ninth aspects of the invention may include any of the features, options or possibilities set out elsewhere in this application, including in the other aspects of the invention, the specific description of the embodiments and the drawings.

According to a tenth aspect of the invention, there is provided a device, the device having:
a) a chamber;
b) a channel; and
c) a vent element;
wherein the chamber is connected to the channel, the channel is connected to the vent element and the vent element leads towards the outside of the device.

The vent element may be an element which is separate from the channel and/or the channel walls. The vent element may be applied to the channel, for instance to one or more of the channel walls.

The vent element may allow the passage of and/or be permeable to air and/or other gases.

The vent element may resist the passage of water and/or other liquids. The vent element may prevent the passage of and/or be impermeable to water and/or other liquids.

The vent element may resist the passage of particulate material. The vent element may prevent the passage of and/or be impermeable to particulate matter. The particulate material may be or include: cells, dust, DNA containing material.

The vent element may be hydrophobic. The vent element may be formed of a hydrophobic material. The vent element may include one or more surfaces provided with a hydrophobic coating.

The vent material may be or include polypropylene. The vent material may include a polysulphone based polymer coating.

The vent element may be or include a filter element.

The vent element may be provided in a vent chamber in the device. The vent chamber may be filled by the vent element. The vent chamber may have an inlet from the channel and an outlet to the outside of the device. The outlet may lead directly to the outside of device or may lead via a vent channel to the outside of the device. The vent element is preferably provided across the path between the inlet to the vent chamber and the outlet from the vent chamber. The vent chamber may have a circular cross-section, particularly perpendicular to the axis of the channel and/or the vent channel. The vent chamber may be cylindrical.

The device may have an orientation of use. In the orientation of use, the vent element may be positioned above the channel. In the orientation of use, the vent element may be positioned above the chamber. In the orientation of use, the part of the channel which is connected to the vent element may be vertically orientated. In the orientation of use, the channel may include a further part which is horizontally orientated.

According to a eleventh aspect of the invention, there is provided a method of producing a device, the method including:
forming a recess in one or more components of the device;
forming a channel in one or more components of the device;
providing a vent element in one or more components of the device;
assembling the one or more components to form a chamber from the recess;
wherein the chamber is connected to the channel, the channel is connected to the vent element and the vent element leads towards the outside of the device.
   According to an twelfth aspect of the invention, there is provided a method of controlling the passage of one or more materials between the inside of a device and the outside of a device, the method including:

moving one or more materials from a chamber into a channel connected to the chamber;

moving one or more of the materials from the channel into a vent element connected to the channel;

moving one or more of the materials from the vent element to the outside of the device.

The fluid pressure on the inside of the vent element may be greater than the fluid pressure on the outside of the vent. Preferably the fluid pressure on the inside of the vent element may be greater than the fluid pressure on the outside of the vent when a connection exists between the outside of the device and channel and/or the chamber. Preferably the vent element is under positive pressure from the inside when a connection exists between the outside of the device and the channel and/or the chamber. Preferably any flow of fluid through the vent element is from the inside of the device to the outside of the device.

The method may include a first stage during which the fluid in the channel is at a higher pressure than the pressure on the outside of the vent element. The method may include a first stage in which fluid flows through the channel and flows through the vent element. Preferably the fluid of the first stage is a gas. Preferably the fluid of the first stage is air.

The method may include a second stage during which the fluid in the channel is at a higher pressure than the pressure on the outside of the vent element. The method may include a second stage in which the fluid does not flow through the vent element. Preferably the fluid of the second stage is a liquid. Preferably the fluid of the second stage is water.

The transition from the first stage to the second stage may occur when the boundary between a first fluid and a second fluid reaches the vent element. The transition from the first stage to the second stage may occur when the boundary between a first fluid and a second fluid reaches a hydrophobic material. The boundary may be between a gas as the first fluid and a liquid as the second fluid. The boundary may be between air as the first fluid and water as the second fluid. The method may include a second stage in which fluid flows through the channel

The seventh and/or eighth and/or ninth aspects of the invention may include any of the features, options or possibilities set out elsewhere in this application, including in the other aspects of the invention, the specific description of the embodiments and the drawings.

According to a thirteenth aspect, there is provided a device, the device including a valve.

Preferably the device only has two types of valve. Preferably all of the valves of each of the two types are identical.

The valve may be an open to closed valve, preferably such that the channel the valve is connected to, is open before the valve is activated and is closed after the valve is activated. Preferably all the valves of the open to closed type are identical in terms of component parts and/or volume and/or length and/or height and/or depth and/or meltable material and/or orientation.

The open to closed valve may include a conduit which connects the valve to the channel to be acted on. The conduit may also connect to a valve reservoir, for instance provided with a meltable material, for instance paraffin wax. The valve reservoir may be connected to a further conduit, such as a gas passage. The further conduit may be connected to a further valve reservoir, for instance provided with air.

Preferably the device has an orientation of use, in the orientation of use, the valve being provided above the channel the valve is to act upon. The section of the channel that the valve is to act upon may be horizontal, for instance +/−10⁰, preferably +/−5⁰ and more preferably +/−3⁰. The conduit and/or further conduit may be vertical, for instance +/−10⁰, preferably +/−5⁰ and more preferably +/−3⁰.

A heater may be provided for the valve. The heater may be provided outside of the device, for instance on another component. The heater may directly or indirectly abut a part of the valve.

The transition from the open state to the closed state may be provided by applying heat to the valve. The heat may cause the meltable material to become a liquid. The heat may cause the contents, particularly air, in the further valve reservoir to expand. Expansion of the contents of the further valve reservoir may assist in moving the contents of the valve reservoir into the channel. The transition from open state to the closed state may be provided by removing a heat source after a period during which heat was applied. The removal of the heat source may cause the meltable material to solidify in the channel.

The section of the channel the valve is to act upon may be provided between one or more further sections. One or more of the further sections may be inclined from the horizontal, for instance by more than 45°, preferably by more than 65° and more preferably by more than 80°. One, preferably two, of the further sections are preferably inclined upwards relative to the section. One, preferably two, of the further sections are provided adjacent the section and/or connected directly thereto.

One or more different melting point meltable materials may be used in a device and/or within a single valve. Within a single valve, the different melting point meltable materials may be used within a single valve reservoir or in separate valve reservoirs. The different melting point meltable materials may be mixed with one another, for instance before and/or during and/or after activation of the valve. A lower melting point material and a higher melting point material may be provided. The higher melting point material may be provided with a melting point greater than 90° C., more preferably greater than 95° C.

The valve may be a closed to open valve, preferably such that the channel the valve is connected to, is closed before the valve is activated and is open after the valve is activated. Preferably all the valves of the closed to open type are identical in terms of component parts and/or volume and/or length and/or height and/or depth and/or meltable material and/or orientation.

The closed to open valve may include a valve chamber which is a part of the channel, having an inlet from the channel and an outlet to the channel. The valve chamber may include a meltable element, the meltable element blocking the channel through the valve chamber in the closed state. The meltable material may be paraffin wax. The valve chamber may include a lower chamber section, preferably provided below the channel and/or flow path through the valve chamber.

Preferably the device has an orientation of use, in the orientation of use, the valve chamber being provided in a horizontal section of the channel the valve is to act upon. The section of the channel that the valve is to act upon may be horizontal, for instance +/−10⁰, preferably +/−5⁰ and more preferably +/−3⁰.

A heater may be provided for the valve. The heater may be provided outside of the device, for instance on another component. The heater may directly or indirectly abut a part of the valve.

The transition from the closed state to the open state may be provided by applying heat to the valve. The heat may cause the meltable material to become a liquid. The heat may cause the meltable material to flow from the blocking position into the lower chamber section. The flow of the meltable material into the lower chamber section may open the channel and/or flow path through the valve chamber. Pressure may be applied behind the meltable material to assist its flow. The transition from closed state to the open state may be provided by removing a heat source after a period during which heat was applied. The removal of the heat source may cause the meltable material to solidify in the lower chamber section.

According to a fourteenth aspect, there is provide a method of producing a device, the method including:

forming a channel in one or more components of the device;

forming a valve connected to the channel;

assembling the one or more components to form a device.

According to a fifteenth aspect, the invention provides a method of controlling the passage of one or more materials within a device, the method including:

moving one or more materials from a first location in the device to a second location in the device;

controlling the passage of the one or more materials using a valve.

The thirteenth and/or fourteenth and/of fifteenth aspects of the invention may include any of the features, options or possibilities set out elsewhere in this application, including in the other aspects of the invention, the specific description of the embodiments and the drawings.

Any of the aspects of the invention may include any of the following options, features or possibilities.

The sample may be received from one or more of: a swab, a buccal swab, a cotton swab, a soft swab, a solution, a suspension, an item of clothing, an item placed in the mouth, a cigarette or piece thereof, chewing gum or saliva.

The sample may be a skin sample, blood sample, cell sample, bodily fluid sample, hair sample, saliva sample or sample containing one or more of these.

The sample may be a forensic sample. The sample may be a medical sample.

The analysis may be for diagnostic purposes. The analysis may be for forensic purposes.

The analysis may be for use in the consideration of marker targets, diagnostic assays, disease markers, biobanking applications, STR based targets in transplants, identification of drug resistant microorganisms, blood testing, mutation detection, DNA sequencing, food analysis, pharmogenetics and pharmogenomics, medical fields, biotech fields, in determining familial relationships, paternity testing and pedigree testing in animals.

The analysis may be for use in border control, security or customs situations and/or uses.

The device may be a microfluidic device. The instrument may incorporate a microfluidic device. The device may be a device processing a sample of less than 1 ml, possibly less than 500 μl, possibly less than 250 μl, potentially less than 200 μl, possibly less than 175 μl, possibly less than 50 μl, preferably less than 30 μl, more preferably less than 20 μl, potentially less than 10 μl in one or more steps. The device may be a device processing a fluid, particularly a liquid, of less than 50 μl, preferably less than 30 μl, more preferably less than 20 μl, potentially less than 10 μl in one or more steps.

The device may process and/or contain a fluid, particularly a liquid, of less than 50 μl, preferably less than 30 μl, more preferably less than 20 μl, potentially less than 10 μl in one or more of the following steps: a sample receiving step and/or sample preparation step and/or sample extraction step and/or sample retention step and/or purification step and/or washing step and/or elution step and/or amplification step and/or PCR step and/or denaturing step and/or investigation step and/or electrophoresis step and/or analysis step and/or results output step.

The device may incorporate one or more channels or chambers with a maximum dimension of less than 1000 μm, possible less than 750 μm and preferably less than 550 μm.

The device may incorporate one or more channels or chambers with a maximum dimension of less than 500 μm, possible less than 250 μm and preferably less than 100 μm.

The device may include a chambers provided with one or more reagents. One or more chambers may be so provided. The reagents may be different. The reagents may be in liquid form. The reagents may be provided on and/or in the surface of a solid. The reagents may be provided on and/or in the surface of a solid in gel form. The solid may be one or more beads. The solid may be magnetic. The reagents may be released as a result of a change in conditions. The change in conditions may be a change in temperature and/or a change in pH.

One or more reagents may be provided for cell lysis. One of more reagents may be provided for a selective extraction of DNA containing material from other material. One or more reagents may be provided for washing. One or more reagents may be provided for elution, particularly from the surface of a solid. One or more reagents may be provided for amplification, particularly PCR based amplification. One or more reagents may be provided for denaturing. One or more reagents may be provided for electrophoresis.

Preferably the device has a stored form and a use form. In the use form, the sample to be processed may be loaded into the device. Preferably one or more reagents are pre-loaded into the device and/or are present in the device when in the stored form. One or more reagents may be loaded into the device in the use form.

The device and/or method may include one or more pumps. Preferably the device only includes pumps of a single type. Preferably the pumps of the single type are identical with respect to chamber shape and/or electrode positions and/or electrode materials and/or orientation and/or chamber volume and/or pump electrolyte and/or pump electrolyte concentration.

One or more, preferably all, of the pumps may be electrochemical pumps.

The device may have an orientation of use, preferably one electrode in the pump chamber is provided above the other. The pump chamber may have a height greater than its width. The pump chamber may have a width greater than its depth.

The pump chamber may have an outlet. Preferably the outlet is provided in the upper section of the pump chamber. The upper section may be the upper 20%, preferably 10%, and more preferably 5% of the height of the chamber. The outlet may be in the top wall of the chamber.

The pump chamber may contain NaCl. The molarity of the electrolyte in the pump chamber may be between 0.2M and 3M, preferably 1M+/−15%.

The electrophoresis step and/or electrophoresis cartridge section may be provided with a channel, for instance a capillary for electrophoresis.

The channel may be provided with a matrix. Preferably the matrix resists the passage of elements, the resistance being related to the size of the element. Preferably different size elements migrate through the matrix at different rates, the larger migrating slower.

The channel may be provided with an inert bed of particulate material to form the matrix.

The channel may be provided with a gel, particularly a polymer gel. The channel may be provided with polyhydroacrylamide, polydimethylacrylamide or mixtures there of. The channel may be provided with a cross-linked polymer. The cross-linking of the polymer may be provided in situ.

One or more surfaces of the channel may be treated, for instance with a hydrophilic coating, for instance poly(hydroxyethlacrylamide).

The channel may be provided with a matrix during electrophoresis. The channel may be provided without a matrix prior to electrophoresis, with the matrix being introduced before electrophoresis commences. The matrix or a material for forming the matrix may be stored at a location removed from the channel in which electrophoresis is provided. The matrix or material for forming the matrix may be stored in a chamber. The chamber may be connected by a channel to the channel in which electrophoresis is provided.

The matrix and/or material for forming the matrix may be altered before use in the electrophoresis step. The alteration may be provided before and/or during and/or after the matrix and/or material for forming the matrix is provided in the channel. The alteration may be polymerisation. The alteration may be caused and/or triggered by heating and/or the application of light, such as U/V light. The alteration may be applied to all of the matrix and/or material for forming the matrix or only a part thereof. One or more parts of the matrix may be prevented from alteration, for instance by masking those parts and/or excluding heat and/or excluding light from them.

Preferably the further sample receiving step may include the transfer of a sample from outside the device and/or instrument, to inside the device and/or instrument. The further sample receiving step may receive the sample from a collection device or from a storage device. The further sample receiving step may include the transfer of the sample to a channel or chamber within the device. The sample receiving step may include the transfer of a sample from outside the device and/or instrument, to inside the device and/or instrument. The sample receiving step may receive the sample from a collection device or from a storage device. The sample receiving step may include the transfer of the sample to a channel or chamber within the device.

Particularly in embodiments where one or more of a sample receiving step and/or sample preparation step and/or sample extraction step and/or sample retention step and/or purification step and/or washing step and/or elution step are provided by the instrument and/or device, then the following features may individually and/or in combinations be provided.

The sample preparation step may include contacting the sample with one or more reagents and/or one or more other components. The reagents and/or other component may be used to prepare the sample for one or more of the subsequent steps. The sample extraction step may be part of or separate from the sample preparation step. The sample extraction step may include contacting the sample with one or more reagents and/or components which select the sample component(s) relative to one or more waste components in the sample. The selected sample component(s) may be removed from the waste component(s) and/or the waste component(s) may be removed from the selected sample components. The waste component(s) may flow away from the extraction step. The waste component(s) may be washed away from the extraction step using one or more further reagents and/or components. The sample retention step may be a part of or may be separate from the sample preparation step and/or sample extraction step. The sample retention step may include contacting the sample with one or more reagents and/or components which retain the sample component(s) relative to one or more waste components in the sample. The sample component(s) may be retained on one or more beads. The beads may be magnetic. The retained sample component(s) may be removed from the waste component(s) and/or the waste component(s) may be removed from the retained sample components. The waste component(s) may flow away from the retention step. The waste component(s) may be washed away from the retention step using one or more further reagents and/or components. The waste component(s) may flow past the location of retention. The waste component(s) may be washed away using one or more further reagents and/or components which flow past the location of retention. The retained and/or selected sample may be eluted, preferably with the eluent conveying the retained and/or selected sample to the next step. The purification step may be a part of or may be separate from the sample preparation step and/or sample extraction step and/or sample retention step. The purification step may separate the selected sample components, for instance DNA, from one or more waste components of the sample, for instance cellular material, PCR inhibitors and chemical inhibitors. The washing step may be a part of or may be separate from the sample preparation step and/or sample extraction step and/or sample retention step and/or purification step. The washing step may remove one or more components of the sample from the location of one or more other components of the sample. The elution step may be a part of or may be separate from the sample preparation step and/or sample extraction step and/or sample retention step and/or purification step and/or washing step. The elution step may remove one or more components of the sample from a first form into a second form. The first form may be bound to a surface or substrate, for instance on a bead. The second form may be in a liquid, for instance the eluent.

The amplification step may include contacting the sample with one or more reagents and/or components to cause amplification. The amplification step may include contacting the sample with conditions, preferably of a cyclic nature, to cause amplification. The amplification may be provided by a PCR step.

The denaturing step may prepare the sample for electrophoresis. The denaturing step may include contacting the sample with one or more reagents and/or components. The denaturing step may include contacting the sample with conditions, preferably of a cyclic nature, to cause denaturing.

The investigation step may provide a characteristic for one component of the sample which differs from the characteristic for one or more other components of the sample. The characteristic may be one or more detectable positions and/or one or more signals and/or one or more intensities and/or one or more colours and/or one or more concentrations and/or presence of one or more characteristics and/or absence of one or more characteristics.

The electrophoresis step may be part of or may be separate from the investigation step. The electrophoresis step may include transferring the sample to a start location for electrophoresis and/or a mobility based separation and/or a size based separation. The start location may be in a channel. The electrophoresis step may include one or more voltage conditions. One or more voltage conditions may be used to transfer the sample to the start location. One or more voltage conditions may be used to provide the separation.

The analysis step may establish one or more of the characteristics of the sample. The analysis may interrogate the instrument, particularly the device, and/or may seek a response from the instrument, particularly the device. The analysis may subject the instrument, particularly the device, to an operation, for instance the application of light. The analysis may consider the response to the operation, for instance the light returning.

The analysis step may include one or more operations involving an interaction with the device. The analysis step may include one or more operations not involving an interaction with the device. One or more of the interactions may be electromagnetic interactions.

The analysis step may apply light to the device. The analysis step may receive light from the device. The analysis step may establish the relative position of the elements having a characteristic, for instance an allele having a fluorescent dye. The analysis step may establish the relative size of the elements having a characteristic, for instance an allele having a fluorescent dye. The analysis step may generate one or more results. The light may be of visible and/or non-visible wavelengths. The results output step may display the one or more results from the analysis step and/or a processed form thereof.

The results output step may transmit the one or more results from the analysis step and/or a processed form thereof to a remote location. The results output step may compile the one or more results into a transmission form. The transmission may be via a telecommunications network. The results may be provided in a format compatible with one or more software applications, for instance one or more software applications for

The results output step may be followed by a further processing step. The further processing may interpret the results to provide further results. The further processing step may analyse the results to provide a DNA profile for the sample. The further processing step may provide an indication of a match between the sample and a database record of a sample. The further processing step may be provided at a location remote from the instrument. The further processing step may be provided at a location connected to the instrument, at least part of the time, by a telecommunications network. The further processing step may return to the instrument and/or a computer, preferably within 200 m of the site of the instrument, the further processed results.

The results may be processed on the instrument to give processed results. The processed results may extract from the results the signals, sections of signals or positions attributable to a characteristic being analysed for, such as an allele. The results and/or processed results may be provided to the results output step.

According to a sixteenth aspect of the invention, there is provided an instrument for analysing a sample, the instrument including:

one or more sample processors;

electronics for operating the sample processors.

According to a seventeenth aspect of the invention, there is provided a device, for processing a sample, the device including:

one or more sample processors.

According to a eighteenth aspect of the invention, there is provided a method of producing a device, the method including:

forming one or more sample processors;

providing electronics for operating the sample processors.

According to a nineteenth aspect of the invention, there is provided a method of analysing a sample, the method including:

applying one or more process steps to the sample;

obtaining one or more results from the method.

The instrument may provide some of a set of process steps and/or sample processors. One or more process steps and/or sample processors may be provided separately from the instrument. The device may provide some of a set of process steps and/or sample processors. One or more process steps and/or sample processors may be provided separately from the device. The process steps and/or sample processors may include a sample receiving step and/or sample preparation step and/or sample extraction step and/or sample retention step and/or purification step and/or washing step and/or elution step and/or further sample receiving step and/or amplification step and/or PCR step and/or denaturing step and/or investigation step and/or detection step and/or electrophoresis step and/or analysis step and/or results output step. The process steps and/or sample processors may include a sample receiving step and/or sample preparation step and/or sample extraction step and/or sample retention step and/or purification step and/or washing step and/or elution step provided separately from the instrument. The process steps and/or sample processors may include a sample receiving step and/or sample preparation step and/or sample extraction step and/or sample retention step and/or purification step and/or washing step and/or elution step provided separately from the device.

The instrument may provide an integrated set of process steps and/or sample processors. The process steps and/or sample processors may include a sample receiving step and/or sample preparation step and/or sample extraction step and/or sample retention step and/or purification step and/or washing step and/or elution step and/or further sample receiving step and/or amplification step and/or PCR step and/or denaturing step and/or investigation step and/or detection step and/or electrophoresis step and/or analysis step and/or results output step.

The instrument may provide for a first and a second amplification and/or PCR step. The first amplification and/or PCR step may include a lower number of amplification cycles and/or have a shorter duration than the second. The first amplification and/or PCR step may provide a first amplification product. The first amplification product may be analysed in the chamber where it is amplified and/or before the second amplification product is analysed. The instrument may include a light source and/or optics and/or detector for analysis of the first amplification product. The instrument may include a light source and/or optics and/or detector which are separate from a light source and/or optics and/or detector which are used to analyse the second amplification product. One or more components may be shared.

One or more negative and/or positive controls may be analysed by the instrument, potentially in parallel to a sample. The same or a different cartridge may be used for the control(s).

The instrument may provide an integrated set of process steps and/or sample processors which progresses from start to finish without user intervention. The start may be the loading of the sample into the instrument, for instance loading the sample into the cartridge and/or loading the cartridge into the instrument. The finish may be the amplification step and/or PCR step and/or denaturing step and/or investigation step and/or electrophoresis step and/or analysis step and/or results output step.

One or more of the steps, and preferably each step, includes one or more operations checks. The operation check may determine whether or not the step occurred correctly, for instance in terms of duration and/or temperature and/or pressure and/or activation. One or more of the steps, and preferably each step, includes a position check. The position check may confirm the orientation and/or alignment and/or horizontal alignment and/or vertical alignment and/or lateral alignment of one component relative to another component.

The instrument may provide one or more of the process steps and/or sample processors on a device, such as a cartridge.

The instrument may receive and/or process one or more devices, such as cartridges. The cartridges may be of the same type. One or more of the cartridges may be of a different type to one or more of the other cartridges. Two or more devices, such as cartridges, may be processed by the instrument simultaneously. Two or more devices, such as cartridges, may be being processed at the same time. At that same time, the devices, such as cartridges, may be at the same or different parts of their processing.

The device may be a single sample only device. The device may be a multi-sample device. The device may provide duplicate channel and/or chamber structures and/or arrangements to process the multiple samples. The device may be a single use only device. The device may be discarded or stored after the single use. The whole and/or a part of the device may be discarded and/or stored after use, such as a single use.

Preferably the process steps and/or sample processors of a further sample receiving step and/or amplification step and/or PCR step and/or denaturing step and/or investigation step and/or electrophoresis step and/or analysis step may be performed on or in the device. The process steps and/or sample processors of a sample receiving step and/or sample preparation step and/or sample extraction step and/or sample retention step and/or purification step and/or washing step and/or elution step and/or further sample receiving step and/or amplification step and/or PCR step and/or denaturing step and/or investigation step and/or electrophoresis step and/or analysis step may be performed on or in the device.

Preferably the process steps and/or sample processors of a sample receiving step and/or sample preparation step and/or sample extraction step and/or sample retention step and/or purification step and/or washing step and/or elution step outside of the device and/or outside of the instrument. Some process steps and/or sample processors may be performed outside of the device but by the instrument.

The process steps and/or sample processors of an analysis step and/or a results output step may be performed on or in the instrument outside of the device.

The analysis step may be partly performed on or in the device and partly performed on or in the instrument aside from the device.

The instrument may provide a set of process steps and/or sample processors which take a time period of less than 300 minutes from start to finish. Preferably the time period is less than 240 minutes, more preferably less than 180 minutes and more preferably less than 150 minutes. The instrument may provide a set of process steps and/or sample processors which take a time period of greater than 30 minutes, preferably greater than 45 minutes and more preferably greater than 60 minutes. The start may be the loading of the sample into the instrument, for instance loading the sample into the cartridge and/or loading the cartridge into the instrument. The finish may be the completion of the investigation step and/or electrophoresis step and/or analysis step and/or results output step.

The instrument may provide a time period of less than 6 minutes from start to finish for the sample receiving step and/or further sample receiving step. Preferably the time period is less than 4 minutes, more preferably less than 3 minutes and more preferably less than 2 minutes. The instrument may provide a set of process steps and/or sample processors from start to finish for the sample receiving step and/or further sample receiving step which take a time period of greater than 10 seconds, preferably greater than 20 seconds and more preferably greater than 30 seconds.

The method and/or overall process and/or instrument may provide a time period from start to finish for the sample preparation step. The sample preparation step may include one or more of an extraction step and/or a sample retention step and/or a purification step and/or a washing step and/or an elution step. The time period may be less than 40 minutes from start to finish, preferably a time period of less than 30 minutes, more preferably less than 25 minutes and still more preferably less than 20 minutes. The time period may be greater than 10 minutes, preferably greater than 15 minutes and more preferably greater than 18 minutes. The start may be defined by the sample leaving the chamber or location into which the sample is loaded. The finish may be the completion of the loading of the sample into an amplification step.

The instrument may provide a time period from start to finish for the amplification step. The amplification step may include a PCR step. The time period may be less than 150 minutes from start to finish, preferably a time period of less than 120 minutes, more preferably less than 90 minutes and still more preferably less than 70 minutes, potentially less than 50 minutes, potentially less than 40 minutes or potentially less than 30 minutes. The time period may be greater than 30 minutes, preferably greater than 40 minutes and more preferably greater than 50 minutes. The start may be defined by the sample entering the chamber or location at which amplification is provided. The finish may be defined by the sample leaving the chamber or location at which amplification has been provided.

The instrument may provide a time period from start to finish for the investigation step and/or electrophoresis step. The investigation step and/or electrophoresis step may include a denaturing step and/or an analysis step. The time period may be less than 45 minutes from start to finish, preferably a time period of less than 30 minutes, more preferably less than 20 minutes and still more preferably less than 18 minutes. The time period may be greater than 8 minutes, preferably greater than 10 minutes and more preferably greater than 12 minutes. The start may be defined by the sample entering the chamber or location at which denaturation is provided. The start may be defined by the sample entering the channel in which electrophoresis is provided. The start may be defined by the sample entering the channel in which the investigation step is provided. The finish may be defined by the receipt of the last signal from investigation step and/or the electrophoresis step and/or from the channel by the light detector. The finish may be defined by the electrophoresis voltage being turned off.

The instrument may include a housing. The housing may have a volume of less than 300,000 cm³, potentially less than 250,000 cm³, preferably less than 200,000 cm³, possibly less than 175,000 cm³, preferably less than 125,000 cm³, more preferably less than 75,000 cm³ and ideally less than 50,000 cm³.

The instrument may have a maximum height of 100 cm, preferably 76 cm and more preferably 65 cm and possibly 50 cm.

The instrument may have a maximum depth of 75 cm, preferably 65 cm and more preferably 55 cm and still more preferably 45 cm.

The instrument may have a maximum width of 50 cm, preferably 45 cm, possibly 40 cm and even possibly 30 cm

The instrument may occupy an area on the surface on which the instrument stands, the maximum area occupied may be less than 2500 cm², preferably less than 2000 cm², more preferably less than 1750 cm², possibly less than 1500 cm² and even possibly less than 1000 cm².

The instrument may weigh less than 20 kg. The instrument may weigh less than 10 kg.

The instrument may be portable. The casing of the instrument may be provided with one or more carrying handles.

The instrument may require only a single connection to a power supply of 110 to 240V and/or 50 Hz. The instrument may require a two or three pin electrical plug for the power supply. The instrument may be provided with a portable power supply, such as a battery based power supply. The portable power supply may be the only power supply for the instrument. The portable power supply may be a backup power supply for the instrument.

The sample may be received from one or more of: a swab, a buccal swab, a cotton swab, a soft swab, a solution, a suspension, an item of clothing, an item placed in the mouth, a cigarette or piece thereof, chewing gum, one or more hairs, a bone sample, a tissue sample or saliva. The sample may be received in solution and/or suspended in a liquid.

The sample receiving step may include the transfer of a sample from outside the device and/or instrument, to inside the device and/or instrument. The sample receiving step may receive the sample from a collection device or from a storage device. The sample receiving step may include the transfer of the sample to a channel or chamber within the device.

The sample preparation step may include contacting the sample with one or more reagents and/or one or more other components. The reagents and/or other component may be used to prepare the sample for one or more of the subsequent steps.

The sample extraction step may be part of or separate from the sample preparation step. The sample extraction step may include contacting the sample with one or more reagents and/or components which select the sample component(s) relative to one or more waste components in the sample. The selected sample component(s) may be removed from the waste component(s) and/or the waste component(s) may be removed from the selected sample components. The waste component(s) may flow away from the extraction step. The waste component(s) may be washed away from the extraction step using one or more further reagents and/or components.

The sample retention step may be a part of or may be separate from the sample preparation step and/or sample extraction step. The sample retention step may include contacting the sample with one or more reagents and/or components which retain the sample component(s) relative to one or more waste components in the sample. The sample component(s) may be retained on one or more beads. The beads may be magnetic. The retained sample component(s) may be removed from the waste component(s) and/or the waste component(s) may be removed from the retained sample components. The waste component(s) may flow away from the retention step. The waste component(s) may be washed away from the retention step using one or more further reagents and/or components. The waste component(s) may flow past the location of retention. The waste component(s) may be washed away using one or more further reagents and/or components which flow past the location of retention.

The retained and/or selected sample may be eluted, preferably with the eluent conveying the retained and/or selected sample to the next step.

The purification step may be a part of or may be separate from the sample preparation step and/or sample extraction step and/or sample retention step. The purification step may separate the selected sample components, for instance DNA, from one or more waste components of the sample, for instance cellular material, PCR inhibitors and chemical inhibitors.

The washing step may be a part of or may be separate from the sample preparation step and/or sample extraction step and/or sample retention step and/or purification step. The washing step may remove one or more components of the sample from the location of one or more other components of the sample.

The elution step may be a part of or may be separate from the sample preparation step and/or sample extraction step and/or sample retention step and/or purification step and/or washing step. The elution step may remove one or more components of the sample from a first form into a second form. The first form may be bound to a surface or substrate, for instance on a bead. The second form may be in a liquid, for instance the eluent.

The instrument may provide some of a set of process steps and/or sample processors. One or more process steps and/or sample processors may be provided separately from the instrument. The process steps and/or sample processors may include a sample collection step and/or a sample transportation step and/or sample storage step and/or a sample receiving step and/or sample preparation step and/or sample extraction step and/or sample retention step and/or purification step and/or washing step and/or elution step provided separately from the instrument and/or from the device.

The sample collection step may include the collection of the sample from a person. The sample collection step may include the collection of the sample from a location, for instance from a person and/or from a location. The location may be a location to which the sample was transferred from a person.

The sample transportation step may provide for the sample being conveyed by person, post, courier or other delivery methods, for instance from a collection location or storage location. The sample transportation step may be provided under ambient temperature conditions.

The sample storage step may provide for the sample being held at a location for a period of time, for instance between the sample collection step and one or more of the subsequent steps, such as a sample receiving step. The sample storage step may be provided under ambient temperature conditions.

The sample receiving step may include the arrival of a sample in the environments of the instrument and/or device, for instance within 500 m thereof. The sample may arrive due to the collection of the sample from a person. The sample may arrive due to the delivery of the sample from a location at which the sample was collected, for instance from a person and/or from a location. The location may be a location to which the sample was transferred from a person. The sample may arrive by post, courier or other delivery methods.

The sample may arrive and/or be received from one or more of: a solid matrix, a solid matrix containing fibres, paper, a swab, a buccal swab, a cotton swab, a soft swab, a solution, a suspension, an item of clothing, an item placed in the mouth, a cigarette or piece thereof, chewing gum, one or more hairs, a bone sample, a tissue sample or saliva.

The sample may be physically bound to a sample collection device. The sample may be chemically bound to a sample collection device. The sample may be dried onto a sample collection device. The sample collection device may include one or more chemicals and/or reagents.

The sample receiving step and/or sample preparation step and/or sample extraction step and/or sample retention step and/or purification step and/or washing step and/or elution step may be provided with one or more chemicals and/or reagents. The chemicals and/or reagents may buffer the sample during one or more steps. The chemicals and/or reagents may alter the sample during one or more steps, for instance by lysing one or more parts of the sample. The chemicals and/or reagents may detach and/or remove and/or release the sample or a part thereof from the form in which the sample is received from the previous step, for instance from a sample collection device or part thereof.

The sample receiving step and/or sample preparation step and/or sample extraction step and/or sample retention step and/or purification step and/or washing step and/or elution step may be provided with one or more conditions which differ from ambient conditions. The conditions may differ in the temperature compared with ambient and/or the level of gravity compared with ambient, for instance due to heating of the sample and/or a container therefore and/or centrifuging. The conditions may alter the sample during one or more steps. The conditions may detach and/or remove and/or release the sample or a part thereof from the form in which the sample is received from the previous step, for instance from a sample collection device or part thereof.

The sample receiving step may receive the sample attached to a solid, such as a matrix.

The sample receiving step may include the transfer of a sample from outside a container to inside a container. The container may be opened, the sample may be introduced and the container may be closed again. The transfer of a sample may include detaching a part of the sample from another part of the sample, for instance part of the matrix from another part of the matrix. The part of the sample may be detached by punching a part of the matrix out of the rest of the matrix. The part of the sample may be detached by cutting a part of the matrix off the rest of the matrix.

The sample preparation step may include contacting the sample with one or more reagents and/or chemicals and/or other components. The reagents and/or chemicals and/or other components may be used to prepare the sample for one or more of the subsequent steps.

The sample extraction step may be part of or separate from the sample preparation step. The sample extraction step may include contacting the sample with one or more reagents and/or chemicals and/or components which select the sample component(s) relative to one or more waste components in the sample. The selected sample component(s) may be removed from the waste component(s) and/or the waste component(s) may be removed from the selected sample components. The waste component(s) may flow away from the extraction step. The waste component(s) may be washed away from the extraction step using one or more further reagents and/or components. The waste components may be retained by the sample collection device and/or part thereof and/or matrix, for instance with the sample being released there from.

The sample extraction step may include eluting at least a part of the sample, for instance from a sample collection device or part thereof. Preferably the eluent conveys the sample to the next step.

The purification step may be a part of or may be separate from the sample preparation step and/or sample extraction step and/or sample retention step. The purification step may separate the selected sample components, for instance DNA, from one or more waste components of the sample, for instance cellular material, PCR inhibitors and chemical inhibitors.

The washing step may be a part of or may be separate from the sample preparation step and/or sample extraction step and/or sample retention step and/or purification step. The washing step may remove one or more components of the sample from the location of one or more other components of the sample.

The elution step may be a part of or may be separate from the sample preparation step and/or sample extraction step and/or sample retention step and/or purification step and/or washing step. The elution step may remove one or more components of the sample from a first form into a second form. The first form may be bound to a matrix or surface or substrate, for instance on a bead. The second form may be in a liquid, for instance the eluent.

The sample preparation step and/or sample extraction step and/or the steps combined may include a purification step followed by an elution step.

The sample preparation step and/or sample extraction step and/or the steps combined may include contacting the sample with water and applying heat to the sample and water.

The sample preparation step and/or sample extraction step and/or the steps combined may include contacting the sample with one or more chemicals, applying mechanical energy, such as agitation to the sample and chemicals, applying heat to the sample and chemicals, applying increased gravity to the sample and chemicals, for instance by centrifuging, removing and/or decanting one or more parts of the mixture, contacting the remainder with one or more chemicals and applying increased gravity to the sample and chemicals, for instance by centrifuging.

The sample preparation step and/or sample extraction step and/or the steps combined may include contacting the sample with one or more chemicals and applying heat to the sample and chemicals.

One or more reagents and/or chemicals and/or other components may be introduced during the sample preparation step and/or extraction step and/or purification step and/or washing step and/or elution step. The container may be opened to provide these reagents and/or chemicals and/or other components. The container may be sealed after they are introduced. The container may be provided with these reagents and/or chemicals.

The sample preparation step and/or sample extraction step may at least partially be provided during the sample transportation step and/or sample storage step, for instance lysis may occur during the sample transportation step and/or sample storage step.

The further sample receiving step may include the transfer of a sample from outside the device and/or instrument, to inside the device and/or instrument. The further sample receiving step may receive the sample from a transport device and/or from a storage device. The transport and/or storage device may be a container. The container may be openable, for instance to provide the container with the sample. The container may be sealable, for instance to retain the sample in the container and/or to exclude contamination from container and/or sample. The container may be provided with one or more chemicals and/or reagents. The container may have one or more chemicals and/or reagents introduced into it. The further sample receiving step may include the transfer of the sample to a channel or chamber within the device, for instance by connecting the container to the device.

Particularly in embodiments where one or more of a sample receiving step and/or sample preparation step and/or sample extraction step and/or sample retention step and/or purification step and/or washing step and/or elution step are provided by the instrument and/or device, then the following features may individually and/or in combinations be provided.

The sample may be received from one or more of: a swab, a buccal swab, a cotton swab, a soft swab, a solution, a suspension, an item of clothing, an item placed in the mouth, a cigarette or piece thereof, chewing gum, one or more hairs, a bone sample, a tissue sample or saliva. The sample may be received in solution and/or suspended in a liquid. The sample receiving step may include the transfer of a sample from outside the device and/or instrument, to inside the device and/or instrument. The sample receiving step may receive the sample from a collection device or from a storage device. The sample receiving step may include the transfer of the sample to a channel or chamber within the device. The sample preparation step may include contacting the sample with one or more reagents and/or one or more other components. The reagents and/or other component may be used to prepare the sample for one or more of the subsequent steps. The sample extraction step may be part of or separate from the sample preparation step. The sample extraction step may include contacting the sample with one or more reagents and/or components which select the sample component(s) relative to one or more waste components in the sample. The selected sample component(s) may be removed from the waste component(s) and/or the waste component(s) may be removed from the selected sample components. The waste component(s) may flow away from the extraction step. The waste component(s) may be washed away from the extraction step using one or more further reagents and/or components. The sample retention step may be a part of or may be separate from the sample preparation step and/or sample extraction step. The sample retention step may include contacting the sample with one or more reagents and/or components which retain the sample component(s) relative to one or more waste components in the sample. The sample component(s) may be retained on one or more beads. The beads may be magnetic. The retained sample component(s) may be removed from the waste component(s) and/or the waste component(s) may be removed from the retained sample components. The waste component(s) may flow away from the retention step. The waste component(s) may be washed away from the retention step using one or more further reagents and/or components. The waste component(s) may flow past the location of retention. The waste component(s) may be washed away using one or more further reagents and/or components which flow past the location of retention. The retained and/or selected sample may be eluted, preferably with the eluent conveying the retained and/or selected sample to the next step. The purification step may be a part of or may be separate from the sample preparation step and/or sample extraction step and/or sample retention step. The purification step may separate the selected sample components, for instance DNA, from one or more waste components of the sample, for instance cellular material, PCR inhibitors and chemical inhibitors. The washing step may be a part of or may be separate from the sample preparation step and/or sample extraction step and/or sample retention step and/or purification step. The washing step may remove one or more components of the sample from the location of one or more other components of the sample. The elution step may be a part of or may be separate from the sample preparation step and/or sample extraction step and/or sample retention step and/or purification step and/or washing step. The elution step may remove one or more components of the sample from a first form into a second form. The first form may be bound to a surface or substrate, for instance on a bead. The second form may be in a liquid, for instance the eluent.

The amplification step may include contacting the sample with one or more reagents and/or components to cause amplification. The amplification step may include contacting the sample with conditions, preferably of a cyclic nature, to cause amplification. The amplification may be provided by a PCR step.

The denaturing step may prepare the sample for electrophoresis. The denaturing step may include contacting the sample with one or more reagents and/or components. The denaturing step may include contacting the sample with conditions, preferably of a cyclic nature, to cause denaturing.

The investigation step may provide a characteristic for one component of the sample which differs from the characteristic for one or more other components of the sample. The characteristic may be one or more detectable positions and/or one or more signals and/or one or more intensities and/or one or more colours and/or one or more concentrations and/or presence of one or more characteristics and/or absence of one or more characteristics.

The electrophoresis step may be part of or may be separate from the investigation step. The electrophoresis step may include transferring the sample to a start location for electrophoresis and/or a mobility based separation and/or a size based separation. The start location may be in a channel. The electrophoresis step may include one or more voltage conditions. One or more voltage conditions may be used to transfer the sample to the start location. One or more voltage conditions may be used to provide the separation.

The analysis step may establish one or more of the characteristics of the sample. The analysis may interrogate the instrument, particularly the device, and/or may seek a response from the instrument, particularly the device. The analysis may subject the instrument, particularly the device, to an operation, for instance the application of light. The analysis may consider the response to the operation, for instance the light returning.

The analysis step may include one or more operations involving an interaction with the device. The analysis step may include one or more operations not involving an interaction with the device. One or more of the interactions may be electromagnetic interactions.

The analysis step may apply light to the device. The analysis step may receive light from the device. The analysis step may establish the relative position of the elements having a characteristic, for instance an allele having a fluorescent dye. The analysis step may establish the relative size of the elements having a characteristic, for instance an allele having a fluorescent dye. The analysis step may generate one or more results. The light may be of visible and/or non-visible wavelengths. The results output step may display the one or more results from the analysis step and/or a processed form thereof.

The results output step may transmit the one or more results from the analysis step and/or a processed form thereof to a remote location. The results output step may compile the one or more results into a transmission form. The transmission may be via a telecommunications network. The results may be provided in a format compatible with one or more software applications, for instance one or more software applications for

The results output step may be followed by a further processing step. The further processing may interpret the results to provide further results. The further processing step may analyse the results to provide a DNA profile for the sample. The further processing step may provide an indication of a match between the sample and a database record of a sample. The further processing step may be provided at a location remote from the instrument. The further processing step may be provided at a location connected to the instrument, at least part of the time, by a telecommunications network. The further processing step may return to the instrument and/or a computer, preferably within 200 m of the site of the instrument, the further processed results.

The results may be processed on the instrument to give processed results. The processed results may extract from the results the signals, sections of signals or positions attributable to a characteristic being analysed for, such as an allele. The results and/or processed results may be provided to the results output step.

The instrument may include a housing, such as a casing. A single housing may be provided for the instrument. The housing may enclose one or more, preferably all of: a cartridge location; cartridge to instrument interface; operating electronics for the cartridge; operating electronics for the cartridge to instrument interface; data processor; powers supply; light source; optical system; light detector; computer software; computer hardware; telecommunications unit.

The casing may include one or more removable panels and/or portions, for instance to allow access to components provided within the casing.

The housing may be provided with a door.

The door may be provided with a restraint, such as a latch, to hold the door in the closed position. The door may be held closed by gravity.

The instrument may have an orientation of use, the door may be provided in the upper half of the instrument, preferably the upper third of the instrument, potentially between 50% and 80% of the way up the instrument. The door may be provided in the upper 20% of the height of the instrument. The door may form part of the upper surface of the casing and/or the front surface of the casing. The hinge for the door may be provided on the upper surface of the casing.

The door may be provided with one or more contact switches. The one or more contact switches may be used to control one or more processes and/or steps and/or actions within the device, for instance the availability of a voltage to certain components within the instrument and/or the availability of light from certain components. The one or more contact switches, when open, may isolate and/or prevent operation of one or more components within the instrument, for instance the power supply or one or more parts of the power supply and/or the laser or other light source.

The cartridge location is preferably provided inside the instrument, preferably behind the door. This may be behind the door by being below it vertically and/or behind the door by being behind it horizontally. The cartridge to instrument interface is preferably provided inside the instrument, preferably behind the door.

The door may have an open position which allows access to a work surface within the instrument. The work surface may include the access route, such as a slot, to the cartridge location.

The cartridge location may be a planar location. The cartridge location may extend parallel to the door, for instance parallel +/−10°, more preferably +/−5°.

With a cartridge in the instrument, in the cartridge location, the cartridge may extend parallel to the door, for instance parallel +/−10°, more preferably +/−5°.

The cartridge to instrument interface may be include a planar surface. The cartridge to instrument interface may include a planar surface which extends parallel to the door, for instance parallel +/−10°, more preferably +/−5°. Preferably the planar surface faces the cartridge location.

The cartridge to instrument interface may include one or more components, preferably exposed at the planar surface. The one or more components may include one or more heaters. The one or more components may include one or more coolers. The one or more components may include one or more Peltier effect components. The one or more components may include an actuator. The one or more components may include one or more sensors, for instance temperature sensors. The or one or more magnets may be moved through an aperture in the cartridge to instrument interface. The one or more magnets may include one or more permanent magnets and/or include one or more electro-magnets.

One or more of the heaters may be printed onto the cartridge to instrument interface. One or more of the heaters may have a square face. The cartridge to instrument interface may have an orientation of use, one or more edges of a heater being inclined relative to the horizontal, preferably by 45°+/−5°.

Preferably the actuator provides reciprocating motion. Preferably the actuator is connected to a mounting for a magnet. The magnet may be a permanent and/or electro magnet. The actuator may have a first state. The actuator may have a second state, the magnet being closer to the cartridge location in the second state than in the first state. The magnet may move along an axis perpendicular to the plane of the cartridge location and/or planar surface of the cartridge to instrument interface, +/−10°, more preferably +/−5°.

One or more of the components may have a planar face. Preferably the planar face of a component faces the cartridge location. Preferably the planar face of a component is parallel to the cartridge location and/or to the cartridge to instrument interface, particularly the planar surface thereof. The planar face of a component may be coplanar with the cartridge location. The planar face of a component may be coplanar with the cartridge to instrument interface, particularly the planar surface thereof. The planar face of a component may be raised compared with the adjoining part of the cartridge to instrument interface, potentially the whole of the planar face of the cartridge to instrument interface.

The cartridge to instrument interface may be a printed circuit board.

The cartridge to instrument interface may be connected to the operating electronics on the rear surface thereof.

The operating electronics for the cartridge may include one or more power supplies. Preferably a power supply is connected to the electrical connections for a pump provided in the cartridge, preferably for each pump. The operating electronics for the cartridge may include a controller or controllers allowing separate operation of each pump in the cartridge.

The connection between the pump and the operating electronics for the cartridge may be provided by abutting contacts. The contacts may abut when the cartridge is introduced to the cartridge location and/or when the door of the instrument is closed. The elements of the operating electronics for the cartridge which contact the cartridge and/or elements mounted thereon are preferably mounted on or in the cartridge to instrument interface.

The connection between the pump(s) and the operating electronics for the cartridge may be provided by one or more pins mounted on the cartridge. The one or more pins may be spring loaded. The one or more pins may be partially or fully recessed into a surface of the cartridge, particularly the planar face thereof. The connection may be provided or may be further provided by one or more pins mounted on the cartridge to instrument interface. The one or more pins may be spring loaded. The one or more pins may be partially or fully recessed into a surface of the cartridge to instrument interface, particularly the planar face thereof which opposes the cartridge. The connection may be made when the cartridge is put in the use position.

The connection between the pump(s) and the operating electronics for the cartridge may be provided in a recessed portion of the cartridge. The recessed portion may be provided by a recess in the perimeter of the cartridge, particularly a recess extending in the main plane of the cartridge.

The operating electronics for the cartridge to instrument interface may be, at least in part, mounted on or in the cartridge to instrument interface. The operating electronics for the cartridge to instrument interface may include one or more power supply controllers and/or one or more heater controllers and/or one or more temperature controllers and/or one or more actuator controllers and/or sensor monitors and/or voltage controllers.

The power supply may include a power supply for the pumps provided in the cartridge. The power supply may include a power supply for one or more heaters provided in or on the cartridge to instrument interface. The power supply may include a power supply for one or more coolers provided on or in the cartridge to instrument interface. The power supply may include a power supply for the actuator for a magnet. The power supply may include a power supply for one or more fans, particularly fans for a Peltier heater. The power supply may include a power supply for an electrophoresis step. The power supply may include a power supply for a light source. The power supply may include a power supply for a light detector. The power supply may include a power supply for an optics alignment and/or verification and/or calibration components.

The light source and/or optical system and/or light detector and/or power supply for one or more of these components may be provided in the lower half, preferably lower third of the instrument.

The light source may be a laser. The laser may emit at a wavelength between 480 nm and 520 nm, preferably between 488 nm and 508 nm. The laser may have a power of at least 15 mW, potentially 20 mW, preferably 25 mW, more preferably 35 mW and ideally at least 45 mW.

The light source may be one or more light emitting diodes.

The optical system may receive light from the light source and/or deliver light to a channel and/or receive light from a channel and/or deliver light to a light detector.

The light detector may be a charge coupled device. The light detector may be provided with one or more lenses.

The computer software may be computer software for implementing the interface with the user. The computer software may be computer software for implementing the operation of one or more of the heaters and/or one or more coolers and/or one or more sensors and/or actuator and/or one or more pumps and/or one or more reactions in the cartridge and/or one or more processes in the cartridge.

The data processor may be a computer. The computer hardware may operate the computer software.

The telecommunications unit may be connected to the data processor and/or computer hardware. The telecommunications unit may be configured to connect to a mobile telecommunications network, such as a mobile phone telecommunications network, satellite phone telecommunications network. The telecommunications unit may be configured to connect to the Internet. The telecommunications device may be configured to provide data from the instrument to a remote location, preferably electronically.

A display unit may be provided in the housing. The instrument may have an orientation of use, the display unit may be provided in upper half of the instrument, preferably the upper quarter of the instrument, potentially between 60% and 95% of the way up the instrument. The display unit may be provided in the lower 50% of the instrument.

The display unit may include a touch sensitive screen. The user may input commands to the instrument through the display. The display may include or be a touch sensitive screen. A stylus and/or stylus storage location may be provided near the display. The stylus may be used to input commands. The user may input commands to the instrument through one or more buttons and/or switches.

The display unit and/or a further display unit may provide a visual indication of the progress of the method relative to the total method. The visual indication may relate to the whole of the method being performed by the instrument and/or one or more of the steps in the whole method. An elongated bar which progressively lights up with progress may be used.

The seventeenth and/or eighteenth and/or nineteenth aspects of the invention may include any of the features, options or possibilities set out elsewhere in this application, including in the other aspects of the invention, the specific description of the embodiments and the drawings.

According to a twentieth aspect of the invention, there is provided an instrument for analysing a sample, the instrument including:

one or more sample processors;

electronics for operating the sample processors.

One or more of the sample processors may be provided on a device. The device may be inserted into the instrument. The device may be a cartridge. One or more of the sample processors may be provided on the instrument.

The device is preferably inserted into a device location. The device location is preferably provided inside the instrument, preferably behind a door.

The device to instrument interface is preferably provided inside the instrument, preferably behind the door. The device to instrument interface is preferably provided by the device abutting a component of the instrument, with the device in the device location. A compressive force may be applied to the device and/or to the component of the instrument, for instance to improve contact.

The device may provide all of the drivers for the process steps and/or processors. The device may provide all the elements which move fluids within the process steps and/or processors. The device may provide all the pumps for the process steps and/or processors. The device may provide all of the materials which form the valves and/or seals in the device. The device may provide all the moveable components for the process steps and/or processors, aside from the actuator for the magnet. The device may provide all the reagents for the process steps and/or processors.

The device may have no electrical power sources therein. The device may have no variable magnetic field source therein. The device may have no fluid expansion drivers therein. The device may have no heaters therein. The device may have no coolers therein. The device may have no sensors therein. The device may have no energy sources therein. No material or elements may enter the device from the interface element.

The instrument, particularly the interface element, may provide all of the electrical power sources for the device. The instrument, particularly the interface element, may provide all of the variable magnetic field sources for the device. The instrument, particularly the interface element, may provide all of the fluid expansion drivers, such as heaters, for the device. The instrument, particularly the interface element, may provide all the heaters for the device. The instrument, particularly the interface element, may provide all of the coolers for the device. The instrument, particularly the interface element, may provide all of the energy sources for the device.

The instrument, particularly the interface element, may have no direct contact with the contents of the device. The instrument, particularly the interface element, may provide none of the drivers for the process steps and/or processors. The instrument, particularly the interface element, may provide none of the elements which move fluids within the process steps and/or processors. The instrument, particularly the interface element, may provide none of the pumps for the process steps and/or processors. The instrument, particularly the interface element, may provide none of the materials which form the valves and/or seals in the device. The instrument, particularly the interface element, may provide none of the moveable components for the process steps and/or processors, aside from the actuator for the magnet. The instrument, particularly the interface element, may provide none of the reagents for the process steps and/or processors.

The interaction between the device and the interface element may be limited to radiation of heat into the device and/or conduction of heat into the device and/or one or more electrical contacts and/or a magnetic field passing into or through the device. No substance may enter the device from the instrument, particularly the interface element.

The device location may be a planar location. The device location may extend parallel to the door, for instance parallel +/−10°, more preferably +/−5°.

With a device in the instrument, in the device location, the device may extend parallel to the door, for instance parallel +/−10°, more preferably +/−5°.

The device may be inserted into the instrument by one or more steps.

The insertion may include the step of inserting a section of the device into a slot in the instrument. The slot may be provided in the bottom section of the device location, for instance the lower 25%, preferably lower 15%. The slot may be provided by a component of the instrument and particularly a component of the interface element. The device may be inclined relative to the plane of the slot and/or device location and/or interface element during the insertion.

The section of the device may be the section of the device which includes the amplification step and/or PCR step and/or a chamber for providing amplification and/or PCR.

The slot may be provided between a first element and a second element. The first element and/or second element may include a heater and/or a cooler and/or a fan. The first element and/or second element may include a Peltier device, preferably a Peltier heater. The first element may have two or more positions, particularly with one of the positions providing a larger separation in the slot between the first element and the second element, than one or more of the other positions. The second element may have two or more positions, particularly with one of the positions providing a larger separation in the slot between the first element and the second element, than one or more of the other positions. The first and/or second element may be biased towards a position with a separation in the slot which is less than in one or more of the positions. The positions may vary by the first and/or second elements moving perpendicular to the plane of the device location and/or device. The first and/or second element may be biased towards a position in which the one or both of the opposing faces of the first and second elements abut the device.

A first heater, such as a Peltier device, may be positioned against a second heater, such as a Peltier device. The first and second heaters may be stacked on one another. The first and second heaters may be provided abutting one another, and preferably with one of the first and second heaters abutting the cartridge in use.

One or more or all of the heaters provided in the instrument may be Peltier devices. One or more or all of the heaters provided in the instrument may be infra-red heaters. One or more or all of the heaters provided in the instrument may be resistance heaters. One or more or all of the heaters provided in the instrument may be microwave heaters.

The insertion may include a step in which the device is rotated about an area or axis, particularly after the section of the device has been inserted into the slot. The rotation may cause one or more elements on the device to cooperate with one or more elements on the instrument, particularly the interface element. One or more elements on the device, such as a cartridge, may cooperate with one or more elements on the interface element. The elements may be a male element, such as a dowel, on one of the device or instrument, preferably with a female element, such as a recess, on the other. Some or all of the male elements may be provided on the device. Some or all of the female elements may be provided on the device. Some or all of the male elements may be provided on the instrument. Some or all of the female elements may be provided on the instrument.

The arrangement of the elements may be such that the device can be positioned in the device location given one orientation of the device. The arrangement of the elements may be such that the device can be positioned in the device location given one orientation of the device. The arrangement of the elements may be such that the device cannot be positioned in the device location given one or more orientations of the device.

The cartridge to instrument interface may include a planar surface. The cartridge to instrument interface may include a planar surface which extends parallel to the door, for instance parallel +/−10°, more preferably +/−5°. Preferably the planar surface faces the cartridge location.

The compressive force may be applied to the device and/or to the component of the instrument by the door of the instrument. The door may be provided with one or more elements which abut the device. The one or more elements may be spring loaded.

The compressive load may be applied by one or more structures, such as a clip or a clamp, which have a first position with a first separation and a second position with a second separation, the second separation being smaller than the first separation, a part of the device and/or of the instrument being provided between the separation. Preferably the second position provides a compressive force to the device and/or instrument.

The device to instrument interface may include one or more components, preferably exposed at the planar surface. The one or more components may include one or more heaters. The one or more components may include one or more coolers. The one or more components may include an actuator. The one or more components may include one or more sensors, for instance temperature sensors. The magnet may be moved through an aperture in the device to instrument interface.

One or more of the heaters may be printed onto the device to instrument interface. One or more of the heaters may have a square face. The device to instrument interface may have an orientation of use, one or more edges of a heater being inclined relative to the horizontal, preferably by 45°+/−5°.

Preferably the actuator provides reciprocating motion. Preferably the actuator is connected to a mounting for a magnet. The magnet may be a permanent and/or electro magnet. The actuator may have a first state. The actuator may have a second state, the magnet being closer to the device location in the second state than in the first state. The magnet may move along an axis perpendicular to the plane of the device location and/or planar surface of the device to instrument interface, +/−10°, more preferably +/−5°.

One or more of the components may have a planar face. Preferably the planar face of a component faces the device location. Preferably the planar face of a component is parallel to the device location and/or to the device to instrument interface, particularly the planar surface thereof. The planar face of a component may be coplanar with the device location. The planar face of a component may be coplanar with the device to instrument interface, particularly the planar surface thereof. The planar face of a component may be raised compared with the adjoining part of the device to instrument interface, potentially the whole of the planar face of the device to instrument interface.

One or more of the components may be fixed relative to the interface element and/or device to instrument interface. One or more of the components may have a degree of movement relative to the interface element and/or device to instrument interface, for instance by spring loading the component. The degree of movement may be perpendicular to the plane of the device location and/or interface element. The degree of movement may be parallel to the plane of the device location and/or interface element. The one or more components may be biased towards the device location and/or away from the interface element.

One or more materials may be provided at the device to instrument interface. The material may be thermally conductive. The material may be uniform. The material may be uniform in the direction between the device and the interface element. The material may vary between locations in the plane of the device to instrument interface. One or more of the materials may be a solid, a paste or a liquid. One or more of the materials may include particles or nanoparticles. One or more of the materials may be provided on the device. One or more of the materials may be provided on the instrument. A compressible material may be provided at the device to instrument interface. A layer, for instance a protective layer, may be provided over the one or more materials. The layer may be a peelable layer. Preferably the layer is removed before the device is inserted into the instrument.

The device to instrument interface may be a printed circuit board.

The device to instrument interface may be connected to the operating electronics on the rear surface thereof.

The operating electronics for the device may include one or more power supplies. Preferably a power supply is connected to the electrical connections for a pump provided in the device, preferably for each pump. The operating electronics for the device may include a controller or controllers allowing separate operation of each pump in the device.

The door may have an open position which allows access to a work surface within the instrument. The work surface may include the access route, such as a slot, to the device location. The device location may be a cartridge location. The device location may be a planar location.

The device location may be opposed by a device to instrument interface, for instance a cartridge to instrument interface, which may itself include a planar surface. The device to instrument interface may include one or more components, preferably exposed at the planar surface.

The device may have an insertion position in which it opposes, but is not in contact with the device to instrument interface. The device may have a use position in which it opposes and is in contact with the device to instrument interface.

The device may be mounted on a carrier.

The device may be mounted on the carrier with the carrier outside of the instrument. The device may be mounted on the carrier before use. The device may be removed from the carrier with the carrier outside of the instrument. The device may be removed from the carrier after use.

The device may provide a first support. The first support may be rectilinear in profile. The first support may receive the device. The planar device may be presented to the planar first support.

The device may be connected to the first support by one or more releasable fasteners, such as screw threaded fasteners. The fasteners may interact with a plurality of engagement locations provided on the device, for instance in the corner portions thereof. The releasable fasteners may be retained on the first support. Initial contact may provide for the releasable fasteners engaging with the device. Tightening of the releasable fasteners may draw the device into contact with the support. The contact may be between one or more peripheral portions of the device of the periphery thereof and the first support. The releasable fasteners may hold the device against the first support, but still allow movement of the device away from the first support in response to a force above a certain level. A force, for instance above that level may be applied to move the device from the insertion position to a use position and/or away from the first support. The force may be removed and/or reduced below that level to allow the device to return to the insertion position and/or into contact with the first support.

One or more of the releasable fasteners may be of the following form. The fastener may include a compressible element, such as a spring and in particular a conical spring between a part of the fastener and a part of the first support. The compressible element may be provided on the side of the first support which is the opposing side to that contacted by the device. Tightening of the fastener and/or displacement of the device from the first support may compress the compressible element. Untightening of the fastener and/or the device approaching the first support may decompress the compressible element. The fastener may be provided with a locking nut and/or washer, preferably on the same side of the first support as that which is contacted by the device.

The device may include the structure providing the investigation step and/or detection step and/or electrophoresis step and/or analysis step and/or results output step. The device may not include the structure providing the investigation step and/or detection step and/or electrophoresis step and/or analysis step and/or results output step. The structure providing the investigation step and/or detection step and/or electrophoresis step and/or analysis step and/or results output step may be provided on an element. The element may be separate from the device. The structure may be the channel(s) and/or chamber(s) and/or electrode(s) for the step, for instance for an electrophoresis step.

The element may be made of a different material and/or to a different manufacturing tolerance to the device.

The element may be mounted on the carrier.

The element may be mounted on the carrier with the carrier outside of the instrument. The element may be mounted on the carrier before use. The element may be removed from the carrier with the carrier outside of the instrument. The element may be removed from the carrier after use.

The carrier may provide support and/or protection for the device and/or element.

The carrier may interact with the instrument and/or casing and/or device location and/or device to instrument interface, for instance to position the device and/or element correctly relative to the device to instrument location. The carrier may be held by the user when inserting the device and/or element into the instrument and/or when removing the device and/or element from the instrument.

The carrier may include a second support. The second support may receive the element. The second support may be rectilinear in profile.

The second support may extend in a second direction. The second direction may be perpendicular, +/−15°, to the first direction in which the first support extends. The maximum extent of the second support in the second direction may be less than 50% and even less than 35% of the maximum extent of the first support in the first direction.

The element may be connected to the second support by cooperation of one or more parts of the element with one or more parts of the second support. The one or more parts of the element may be opposing ends of the element. The one or more parts of the second support may be a pair of slots provided in the second element. The element may be inserted into the second support in a first direction and may be removed there from in a second direction which is the reverse of the first direction. The first and/or second directions may be in the plane of the element and/or second support.

A releasable and/or adjustable fastener provided on the second support may engage with a part of the element. For instance, a protrusion may cooperate with a recess.

The device may be connected to the element, for instance to allow the passage of fluid from the device to the element. The connection may be formed by the insertion of the device into the first support and/or the element into the second support. The connection may be a tube, for instance a flexible tube. The carrier may accommodate and/or support the tube. The carrier may include a first aperture through which the tube passes from the device. The aperture may lead to a void within the carrier. The carrier may include a second aperture through which the tube passes from the void to the element. The tube may make a first turn from the plane of the device into the plane of the element, ideally within the carrier. The tube may make a second turn into alignment with a channel within the device, ideally within the carrier.

The carrier, including the device and/or element, may be inserted into the instrument, for instance into a slot, for instance accessed from the work surface. The carrier may be inserted until one or more parts thereof abut one or more parts of the slot and/or work surface and/or instrument and/or casing.

The carrier may be in an insertion position when further inward movement is stopped. The insertion position may provide the device, in opposition to the device to instrument interface, but spaced there from.

The device may be moved from the insertion position to a use position, for instance in which the device contacts the device to instrument interface. The movement to the use position and/or a subsequent step may provide for one or more or all of the following: the application of force to the device to hold it in position against the device to instrument interface; the formation of contact between the element or that part of the device providing the electrophoresis step with a heater board; the formation of electrical contacts between the instrument and one or more electrical components, such as electrochemical pumps, provided on the device; the formation of electrical contacts between the instrument and one or more electrical components, such as electrodes, provided on the element or that part of the device providing the electrophoresis step and/or the deactivation of one or more interlocks, for instance for the power supply or one or more parts thereof or the light source.

The device may be moved from the insertion position to the use position by contact between a displacement element with the device. For instance, a platen may be advanced from a non-contact position to a contact position and then to a displacing position. In the non-contact position, the displacement element may be outside of the slot into which the carrier and cartridge are inserted. In the contact position, the displacement element may be inside the slot and/or in contact with the device. The displacement element may pass through an opening from which the carrier and/or first support is absent, but in which the device is present. In the displacing position, the displacement element may move the device away from the carrier and/or first support. The device is preferably still connected to the carrier and/or first support by the one or more releasable fasteners.

The device may engage one or more elements on the device to instrument interface and/or extending there through, particularly during the transition from insertion position to use position. The elements may be a male element, such as a dowel, on one of the device or instrument, preferably with a female element, such as a recess, on the other. Some or all of the male elements may be provided on the device. Some or all of the female elements may be provided on the device. Some or all of the male elements may be provided on the instrument. Some or all of the female elements may be provided on the instrument. The arrangement of the elements may be such that the device can be positioned in the device location given one orientation of the device. The arrangement of the elements may be such that the device can be positioned in the device location given one orientation of the device. The arrangement of the elements may be such that the device cannot be positioned in the device location given one or more orientations of the device.

The device may be moved from the use position to the insertion position by withdrawal of a displacement element. The displacement element may be withdrawn from a displacing position to a contact position, for instance to thereby allow the device to move from the use position to the insertion position, most preferably due to expansion of a compressible element. The displacement element may be withdrawn from a contact position to a non-contact position, for instance to allow the carrier and/or device to be removed, for instance from the slot.

The device may be capable of movement relative to the carrier when provided on the carrier. The device may be capable of independent lateral and/or vertical and/or horizontal movement relative to the carrier. The element may be capable of movement relative to the carrier when provided on the carrier. The element may be capable of independent lateral and/or vertical and/or horizontal movement relative to the carrier. The movement relative to the carrier may be used to move the device and/or element from a insertion position to a use position or more preferably from a first use position to a second optimised use position. In the optimised use position, the device and/or cartridge is positioned in the optimum position relative to the device to instrument interface and/or element to instrument interface and/or optical system and/or temperature control system.

The relative movement for the device and/or element may be made in response to one or more alignment checks, for instance conducted by the instrument.

The device may include a section providing one or more of a sample receiving step and/or sample preparation step and/or sample extraction step and/or sample retention step and/or purification step and/or washing step and/or elution step and/or amplification step and/or PCR step and/or denaturing step and/or investigation step and/or detection step and/or electrophoresis step and/or analysis step and/or results output step. The device may include a further section providing one or more of the steps not provided by the section. A carrier may be provided for the section and further section. The section and further section may be capable of movement relative to the carrier when provided on the carrier. The section and/or further section may be capable of independent lateral and/or vertical and/or horizontal movement relative to the carrier. The movement relative to the carrier may be used to move the section and/or further section from an insertion position to a use position or more preferably from a first use position to a second optimised use position. In the optimised use position, the device and/or cartridge is positioned in the optimum position relative to the device to instrument interface and/or element to instrument interface and/or optical system and/or temperature control system.

The relative movement for the section and/or further section may be made in response to one or more alignment checks, for instance conducted by the instrument.

Preferably the device may include one or more of a further sample receiving step and/or amplification step and/or PCR step and/or denaturing step and/or investigation step and/or detection step and/or electrophoresis step and/or analysis step and/or results output step. The device may include one or more of a sample receiving step and/or sample preparation step and/or sample extraction step and/or sample retention step and/or purification step and/or washing step and/or elution step and/or further sample receiving step and/or amplification step and/or PCR step and/or denaturing step and/or investigation step and/or detection step and/or electrophoresis step and/or analysis step and/or results output step. The device may be capable of movement relative to the carrier when provided on the carrier. The device may be capable of independent lateral and/or vertical and/or horizontal movement relative to the carrier. The movement relative to the carrier may be provided before and/or after one or more of the steps of a sample receiving step and/or sample preparation step and/or sample extraction step and/or sample retention step and/or purification step and/or washing step and/or elution step and/or further sample receiving step and/or amplification step and/or PCR step and/or denaturing step and/or investigation step and/or detection step and/or electrophoresis step and/or analysis step and/or results output step. The movement relative to the carrier may be provided before a sample extraction step. The movement relative to the carrier may be provided before an investigation step and/or detection step and/or electrophoresis step and/or analysis step and/or results output step. A plurality of different movements may be provided. A first movement may be provided to position and/or align and/or bring into contact the device and the device to instrument interface with respect to one or more of the steps of a sample receiving step and/or sample preparation step and/or sample extraction step and/or sample retention step and/or purification step and/or washing step and/or elution step and/or further sample receiving step and/or amplification step and/or PCR step and/or denaturing step. A second movement may be provided to position and/or align and/or bring into contact the device and the device to instrument interface with respect to one or more of the steps of an investigation step and/or detection step and/or electrophoresis step and/or analysis step and/or results output step

The relative movement for the section and/or further section may be made in response to one or more alignment checks, for instance conducted by the instrument.

The insertion of the carrier into the instrument may provide contact between the element or that part of the device providing the electrophoresis step and the device to instrument interface or a section thereof.

The contact may include one or more protrusions on the device to instrument interface interacting with one or more apertures provided in the carrier and/or second support and/or element or that part of the device providing the electrophoresis step. The contact may result in movement of the element or that part of the device providing the electrophoresis step relative to the carrier.

The contact may include contact between the element, or that part of the device providing the electrophoresis step, with a heated surface or location. The heated surface or location may be in the form of a thermally conductive block or heat sink, preferably provided in contact with one or more heaters. The heated surface or location may provide a planar surface portion. The heated surface or location may contact a planar surface by the channel, for instance by the electrophoresis cartridge section. The heated surface or location may have a planar surface portion whose boundaries match those of the planar surface of the channel, for instance as provided by the electrophoresis cartridge section. The heated surface or location may be bounded by one or more protruding or elevated sections. The protruding or elevated section(s) may surround the heated surface or location and/or the planar surface of the channel. The heated surface or location may be recessed compared with one or more sections provided in proximity thereto. The section(s) may surround the heated surface or location and/or the planar surface of the channel. The channel or electrophoresis cartridge section containing the channel may fit into the heating location. A snug fit may be provided.

The insertion may include the step of inserting a section of the device into a slot within the device location in the instrument. The slot may be provided in the upper section of the device location, for instance the upper 25%. The slot may be provided by a component of the instrument and particularly a component of the interface element. The component of the interface element may be in the form of a pair of supports, such as calipers, which extend partially across each side of the device. The section of the device may be the section of the device which includes the amplification step and/or PCR step and/or a chamber for providing amplification and/or PCR.

The slot may be provided between a first element and a second element. The first element and/or second element may include a heater and/or a cooler and/or a fan. The first element and/or second element may include a Peltier device, preferably a Peltier heater. The first element and/or second element may provide one or more temperature sensors. The first element may have two or more positions, particularly with one of the positions providing a larger separation in the slot between the first element and the second element, than one or more of the other positions. The second element may be fixed in position. The first element may be biased towards a position with a separation in the slot which is less than in one or more of the positions. The positions may be varied by the first element moving perpendicular to the plane of the device location and/or device.

The connection between the pump and the operating electronics for the device location may be provided by abutting contacts. The contacts may abut when the device is introduced to the device location and/or when the door of the instrument is closed. The elements of the operating electronics for the device which contact the device and/or elements mounted thereon are preferably mounted on or in the device to instrument interface.

The operating electronics for the device to instrument interface may be, at least in part, mounted on or in the device to instrument interface. The operating electronics for the device to instrument interface may include one or more power supply controllers and/or one or more heater controllers and/or one or more temperature controllers and/or one or more actuator controllers and/or sensor monitors and/or voltage controllers.

The power supply may include a power supply for the pumps provided in the device. The power supply may include a power supply for one or more heaters provided in or on the device to instrument interface. The power supply may include a power supply for one or more coolers provided on or in the device to instrument interface. The power supply may include a power supply for the actuator for a magnet. The power supply may include a power supply for one or more fans, particularly fans for a Peltier heater. The power supply may include a power supply for an electrophoresis step. The power supply may include a power supply for a light source. The power supply may include a power supply for a light detector. The power supply may include a power supply for an optics alignment and/or verification and/or calibration components.

According to a twenty first aspect of the invention, there is provided a method of producing a device, the method including:

forming one or more sample processors;

providing electronics for operating the sample processors.

According to a twenty second aspect of the invention, there is provided a method of analysing a sample, the method including:

applying one or more process steps to the sample;

obtaining one or more results from the method.

The twentieth and/or twenty first and/or twenty second aspects of the invention may include any of the features, options or possibilities set out elsewhere in this application, including in the other aspects of the invention, the specific description of the embodiments and the drawings.

Various embodiments of the present invention will now be described, by way of example only, and with reference to the accompanying drawings in which:

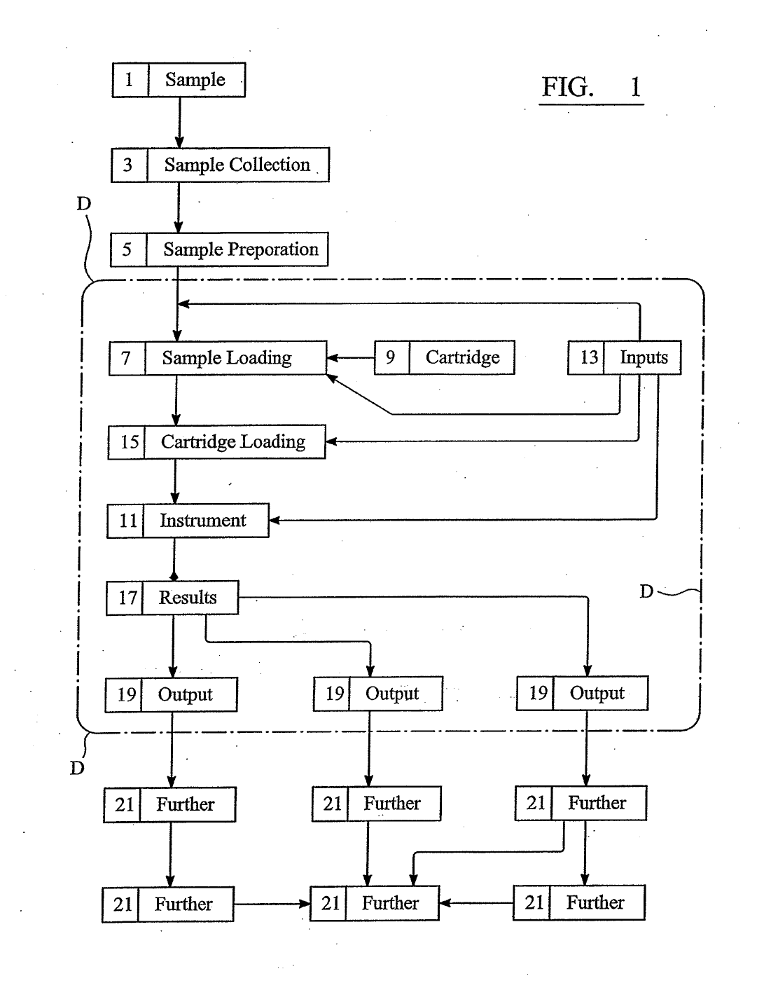

FIG. 1 is a schematic illustration of the stages involved in the consideration of a sample from collection to results;

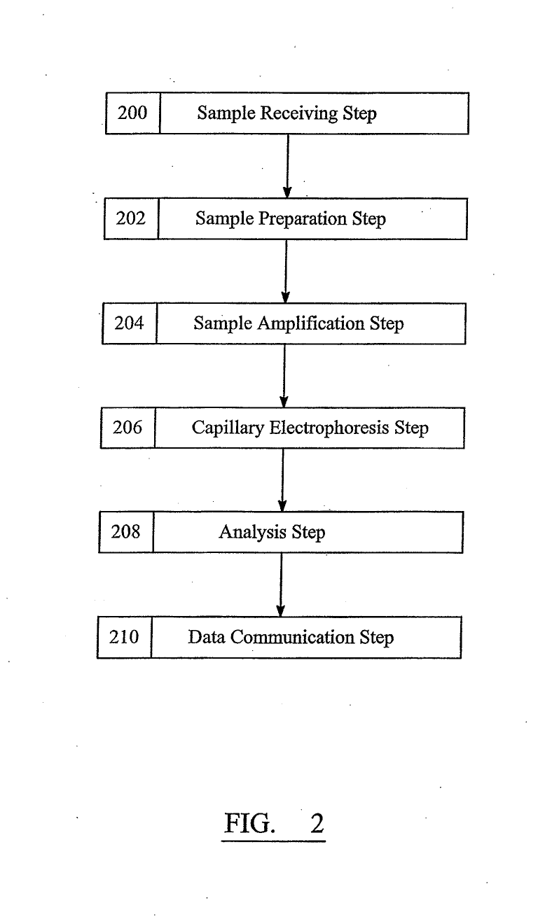

FIG. 2 is a schematic illustration of the key steps provided on or by an instrument;

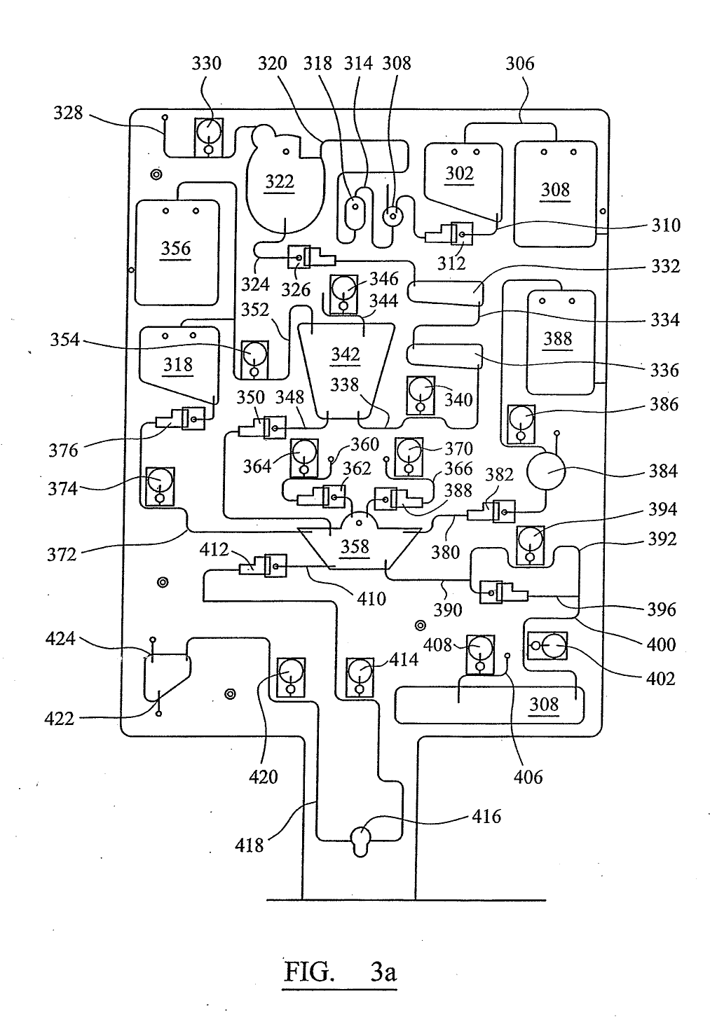

FIG. 3a is a front face view of part of a cartridge;

FIG. 3b is a table of dimensions and volumes for a cartridge and components thereof;

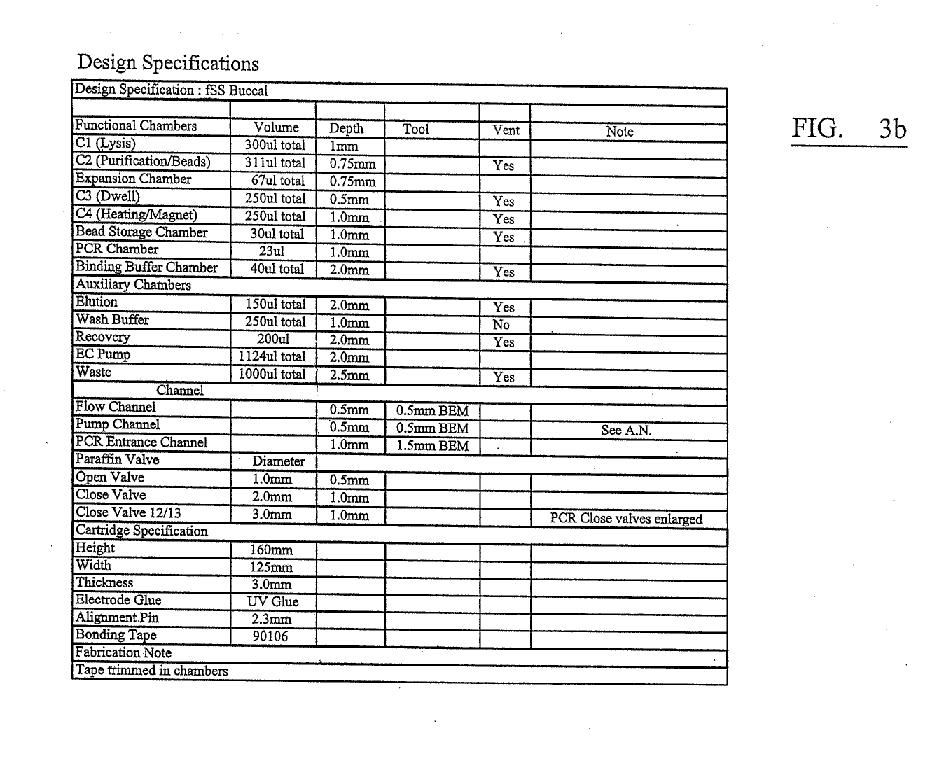

FIG. 4 is a front face view of a further part of the cartridge of FIG. 3a;

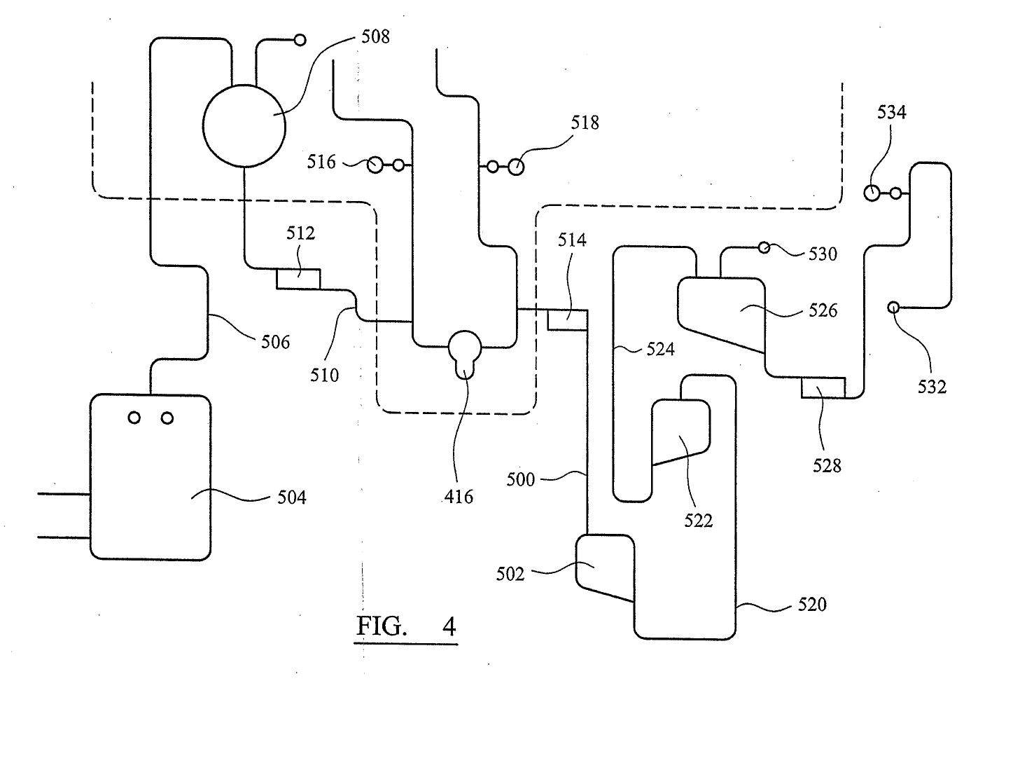

FIG. 5a is a side view of the section of the cartridge of FIGS. 3a and 4 where in joins the electrophoresis cartridge section;

FIG. 5b is a front view of the electrophoresis cartridge section shown in FIG. 5a, with the section of the cartridge omitted;

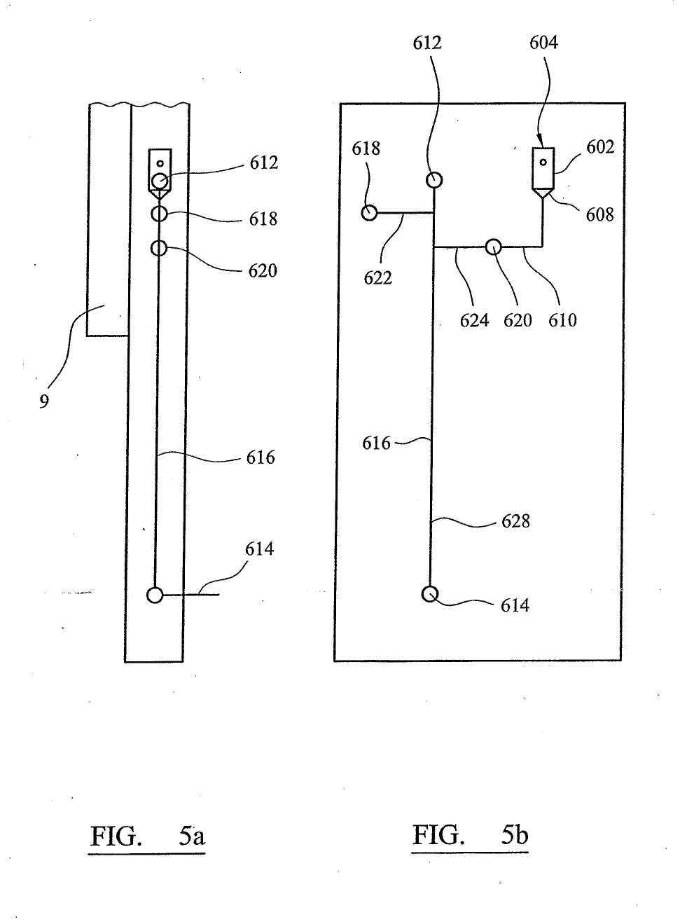

FIGS. 6a to 6e are schematic illustrations of alternative arrangements for contacting the fluid and beads;

FIG. 7 is an illustration of an alternative structure for providing sample to the PCR chamber;

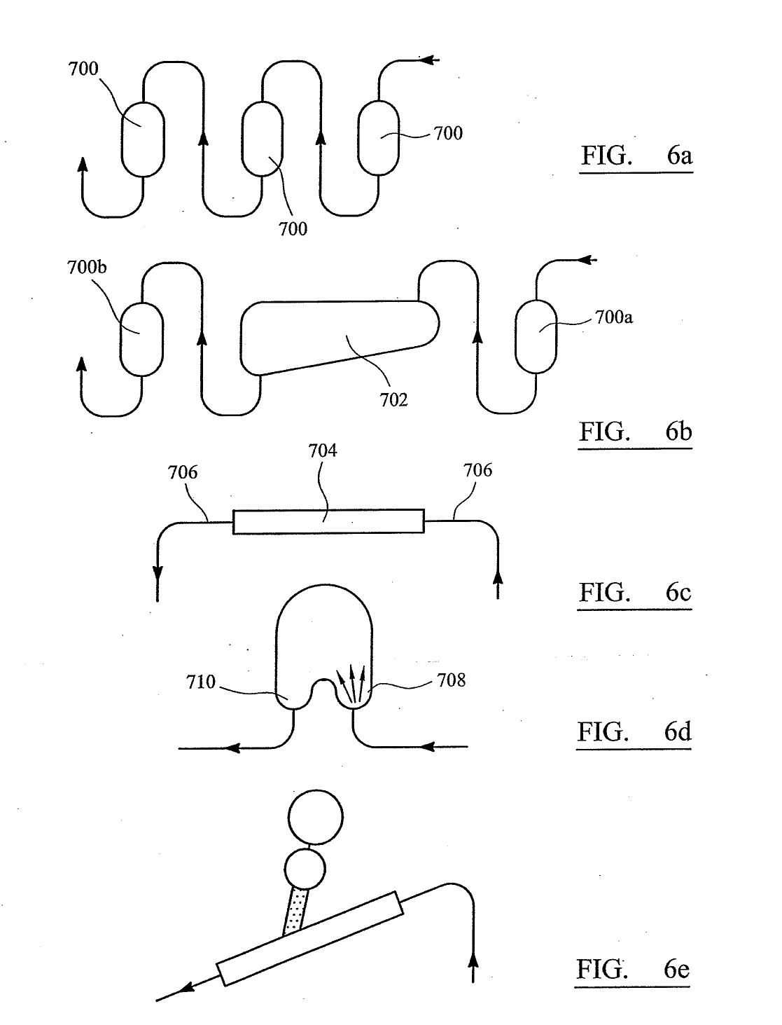

FIG. 8 is a front view of the electrophoresis cartridge section showing an alternative form of injector;

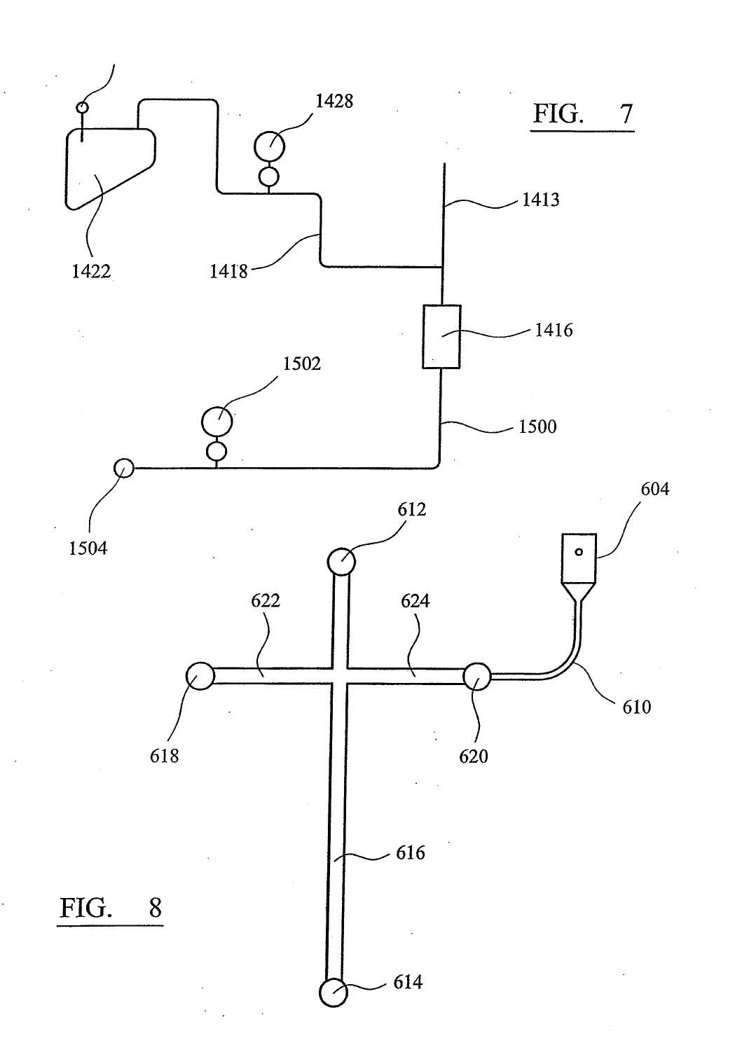

FIG. 9 is a schematic illustration of the parallel PCR chamber arrangement used in providing real time PCR and feedback of the results;

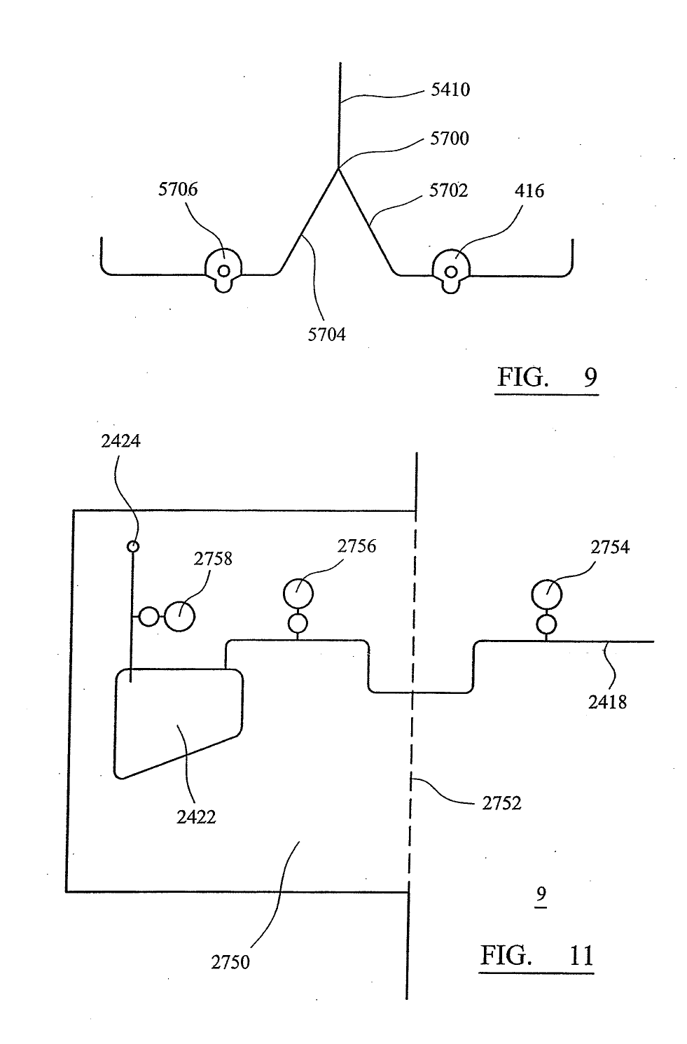

FIG. 10a is an illustration of a closing valve used in the present invention;

FIG. 10b is an illustration of an opening valve used in the present invention;

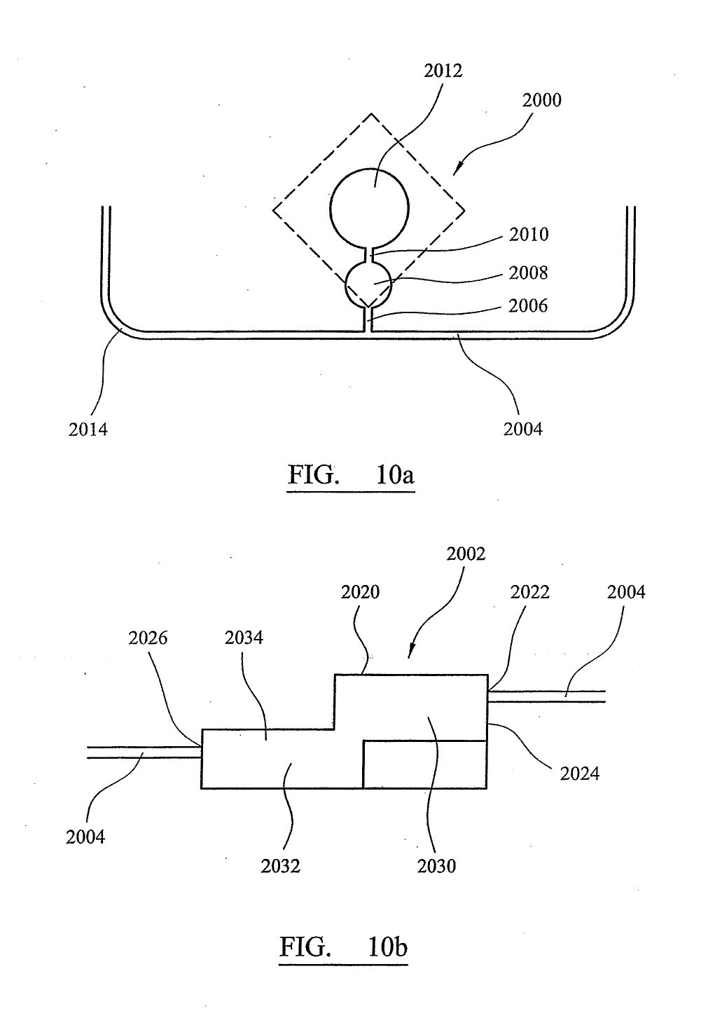

FIG. 11 shows an option for the archiving of a part of the sample handled;

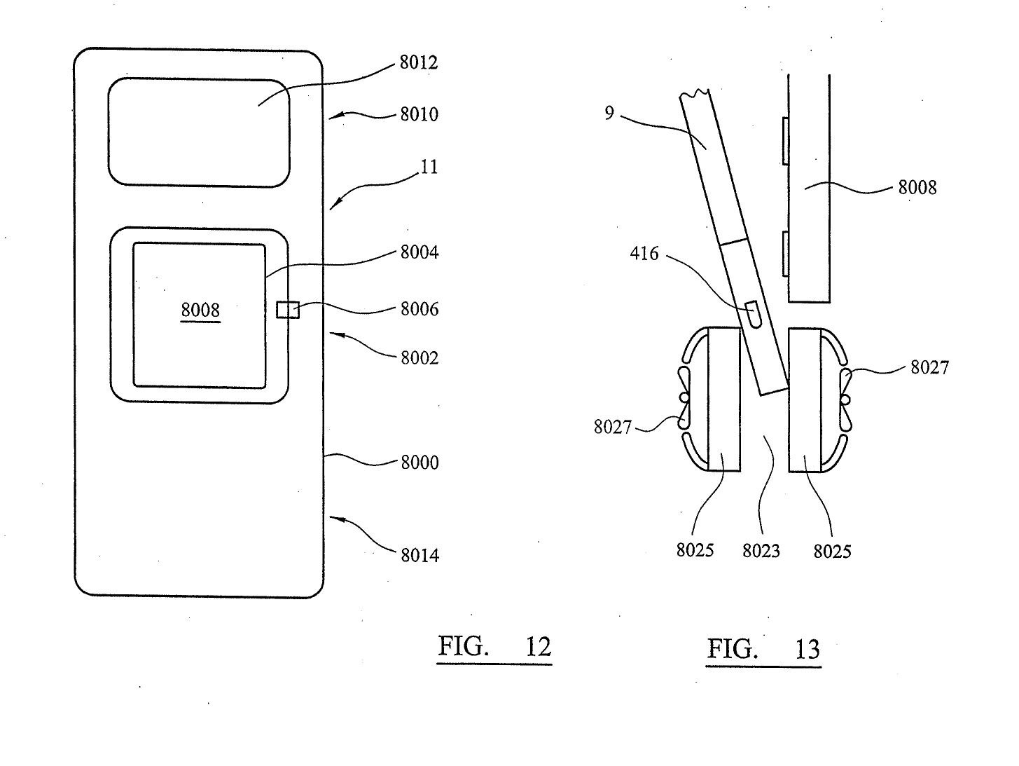

FIG. 12 is a schematic front view of an instrument;

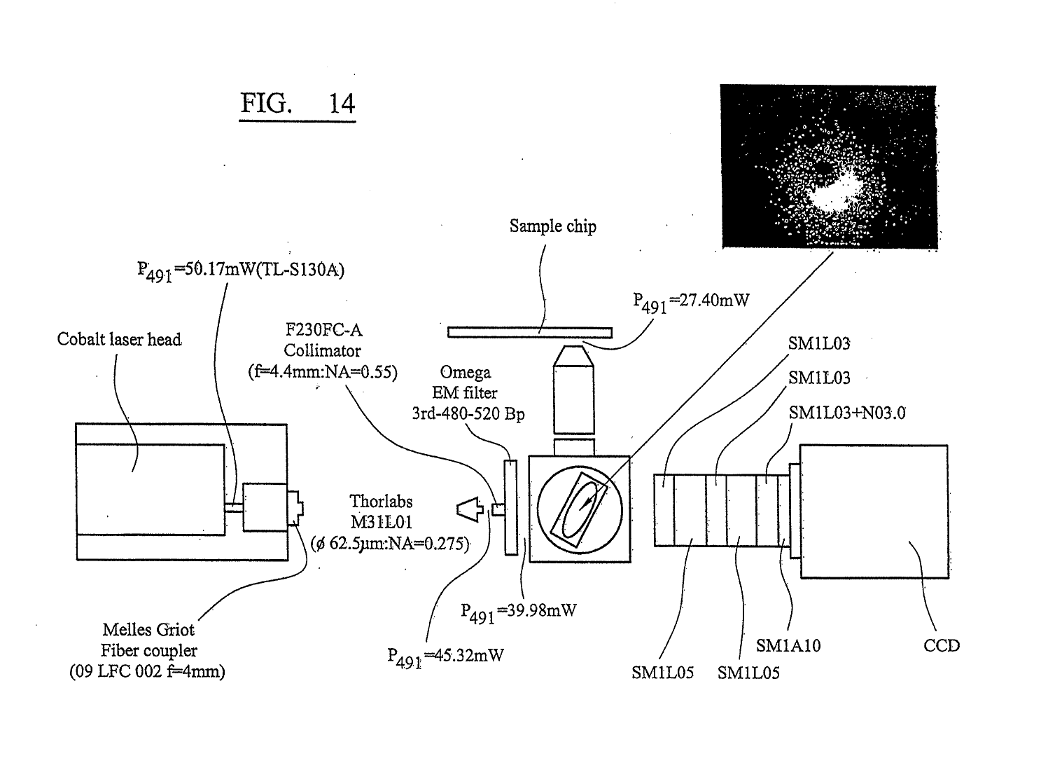

FIG. 13 is a side view showing the insertion of the cartridge into the instrument;

FIG. 14 is a schematic of the light source, optics and detector setup for the electrophoresis section of the instrument;

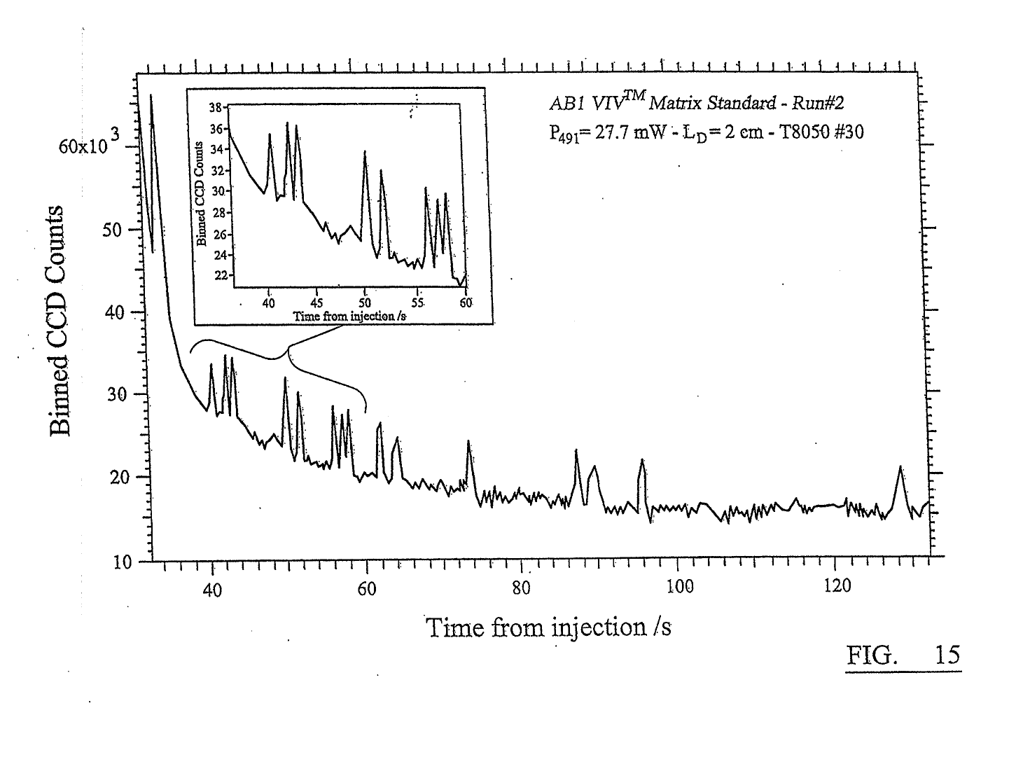

FIG. 15 is an electropherogram showing the variation in signal from the detector setup with time;

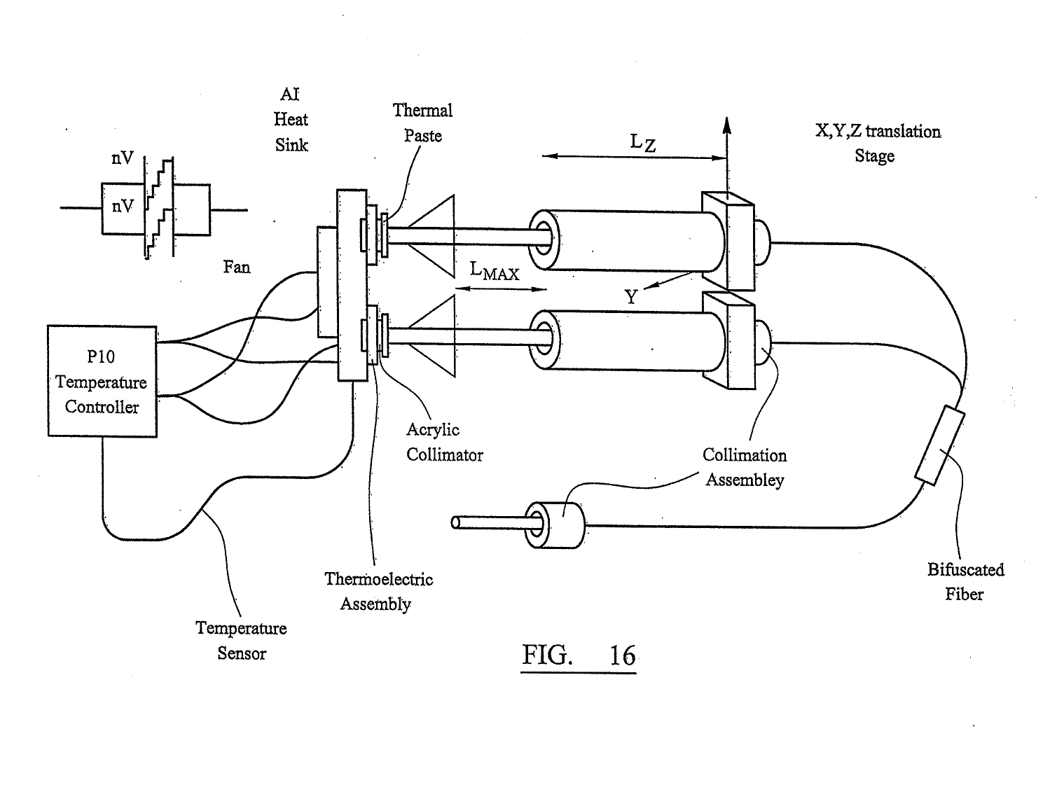

FIG. 16 is a schematic of an example of a system for detecting fluorescence;

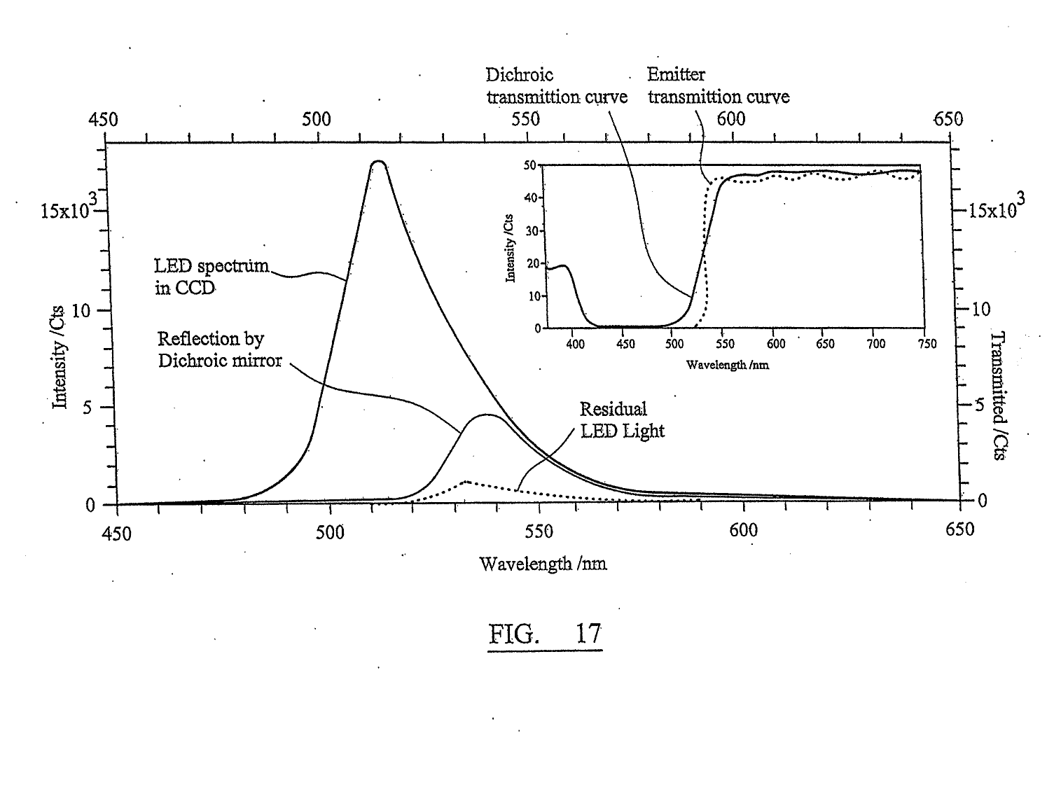

FIG. 17 is a plot of LED spectrum, light reflected, and residual LED light over a range of wavelengths;

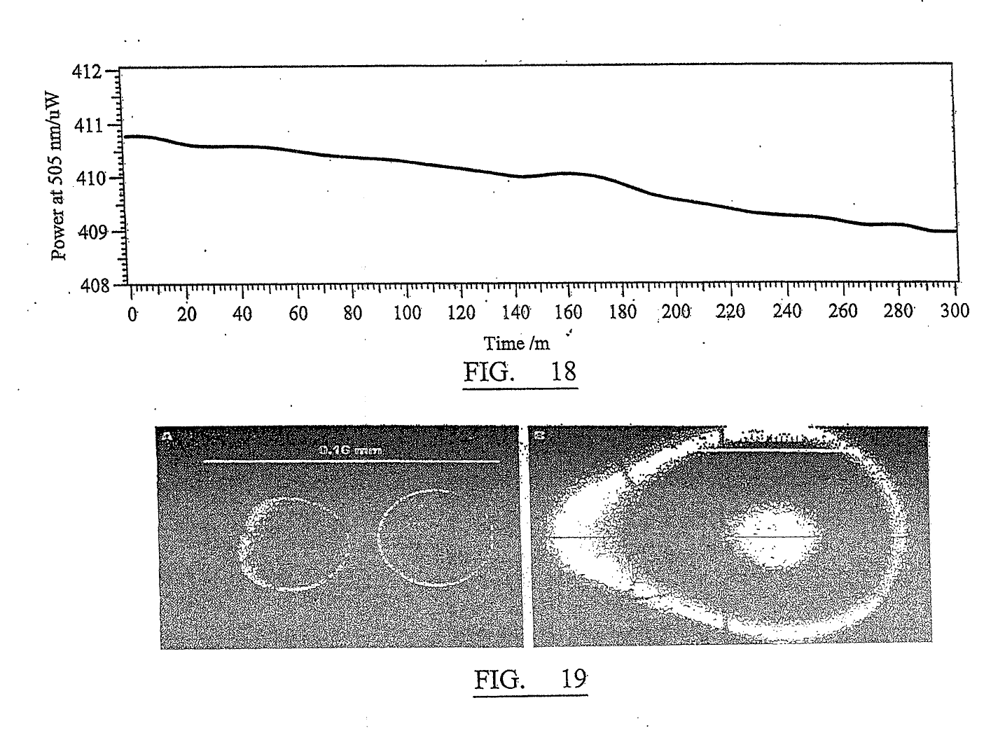

FIG. 18 is a plot of power of the LED-module over time;

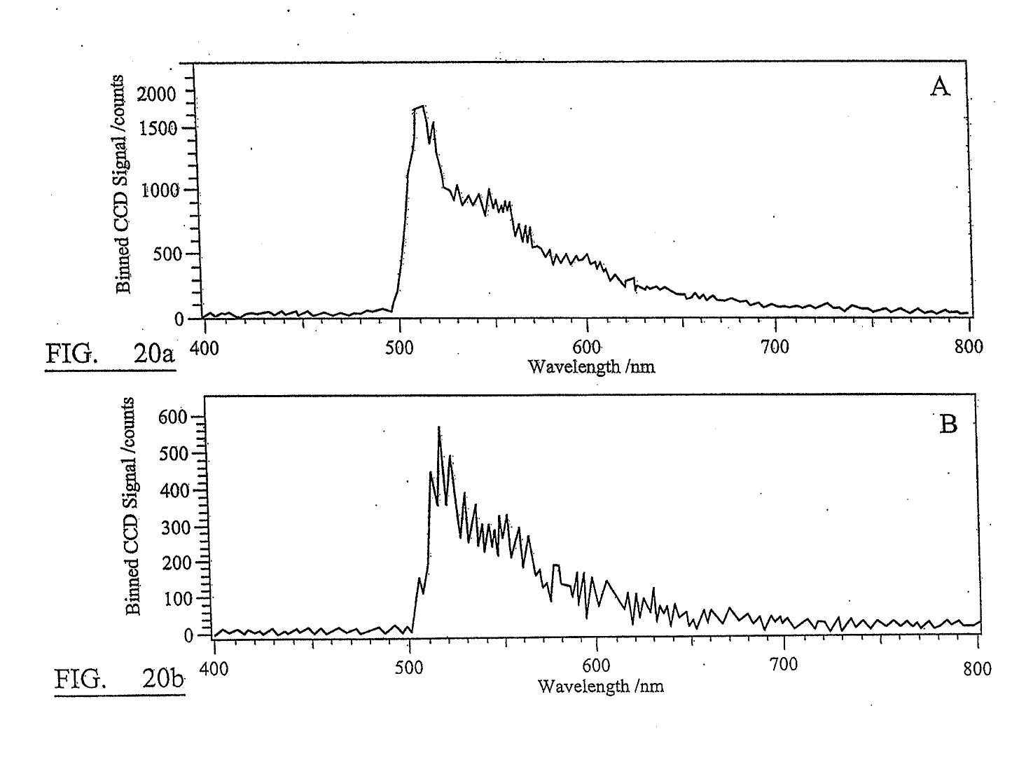

FIG. 19 is an illustration showing beam shape and size as measured by the laser camera;

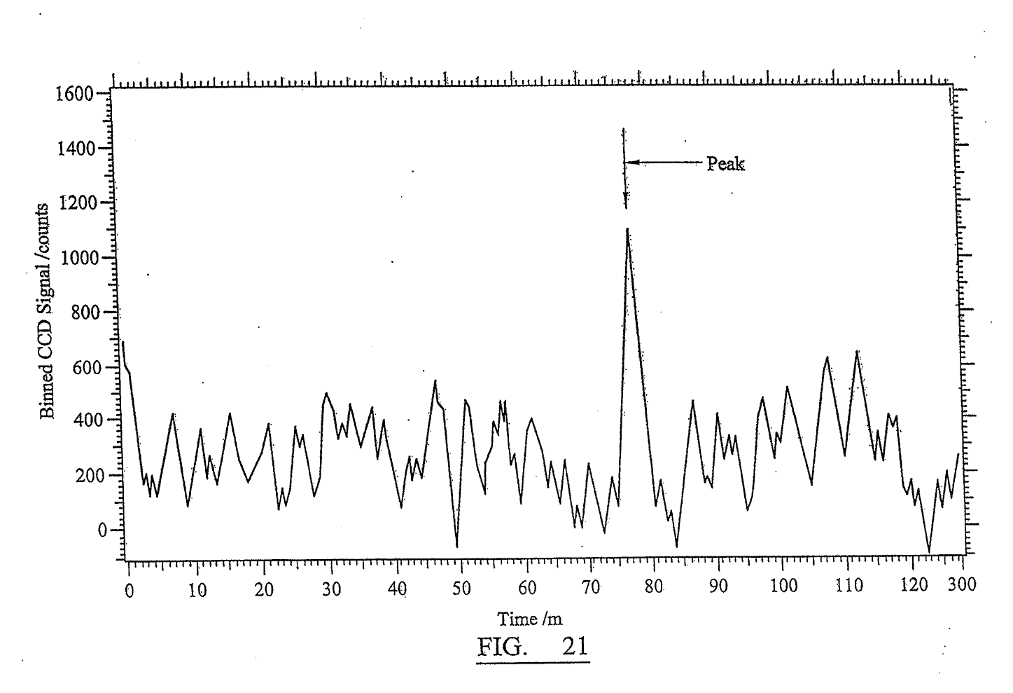

FIGS. 20A and 20B are plots of CCD signal v/s wavelengths for static fluorescence measurements; and

FIG. 21 is a plot of CCD signal v/s time for dynamic fluorescence measurements;

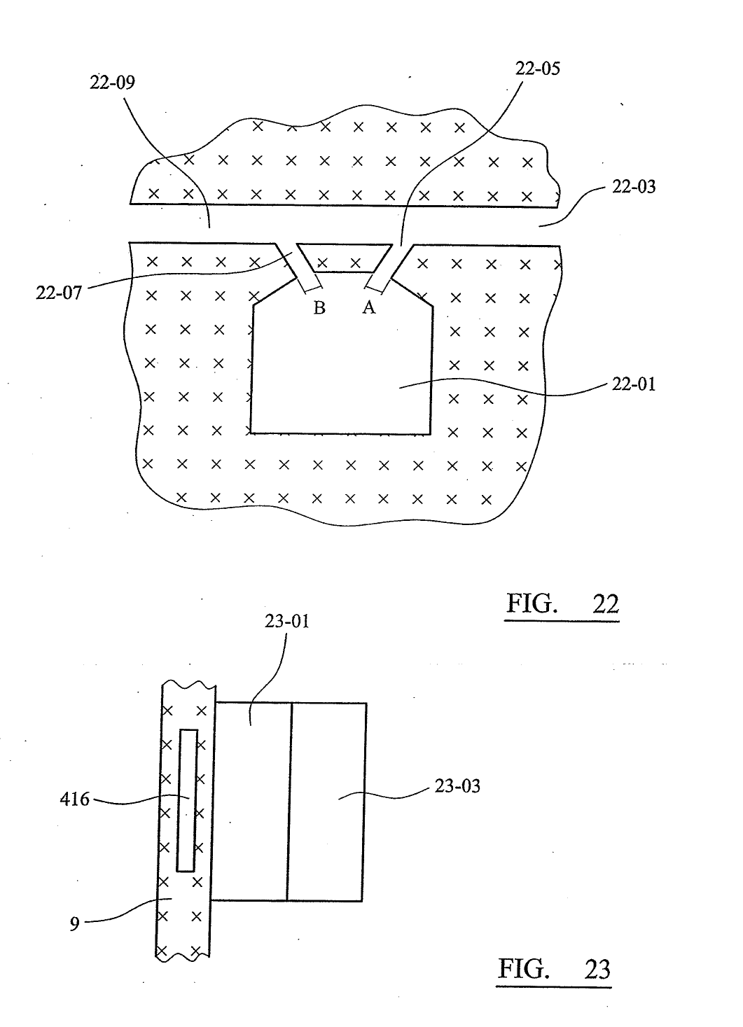

FIG. 22 is an illustration of a PCR chamber;

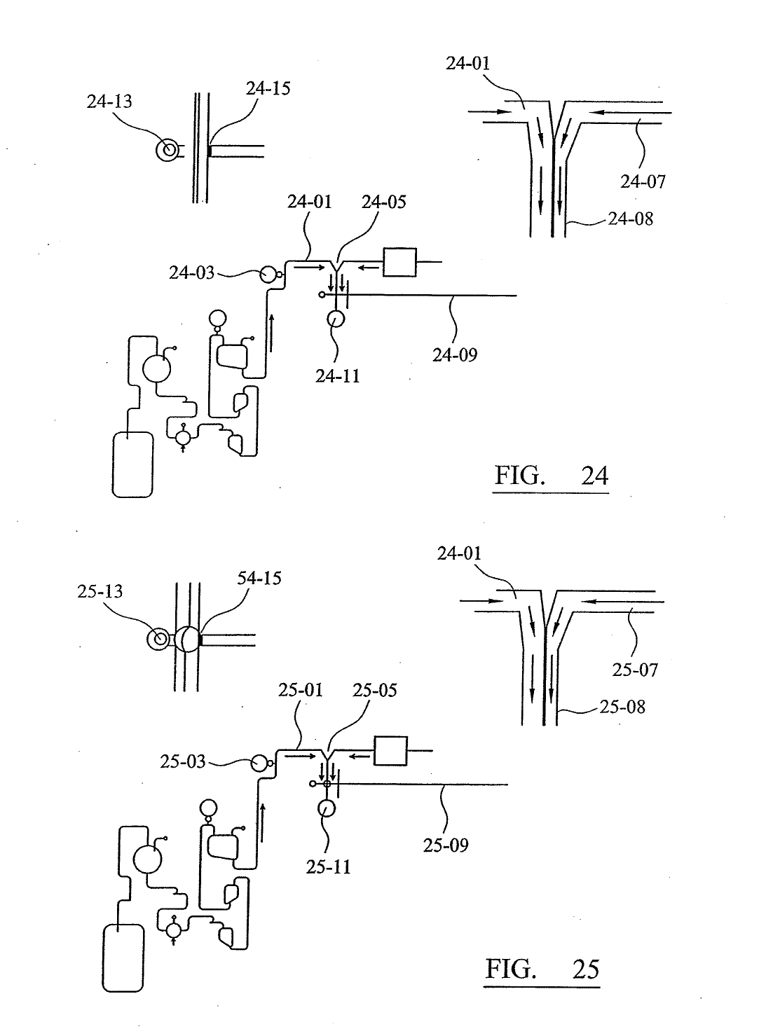

FIG. 23 is an illustration of the position of stacked Peltier effect devices;

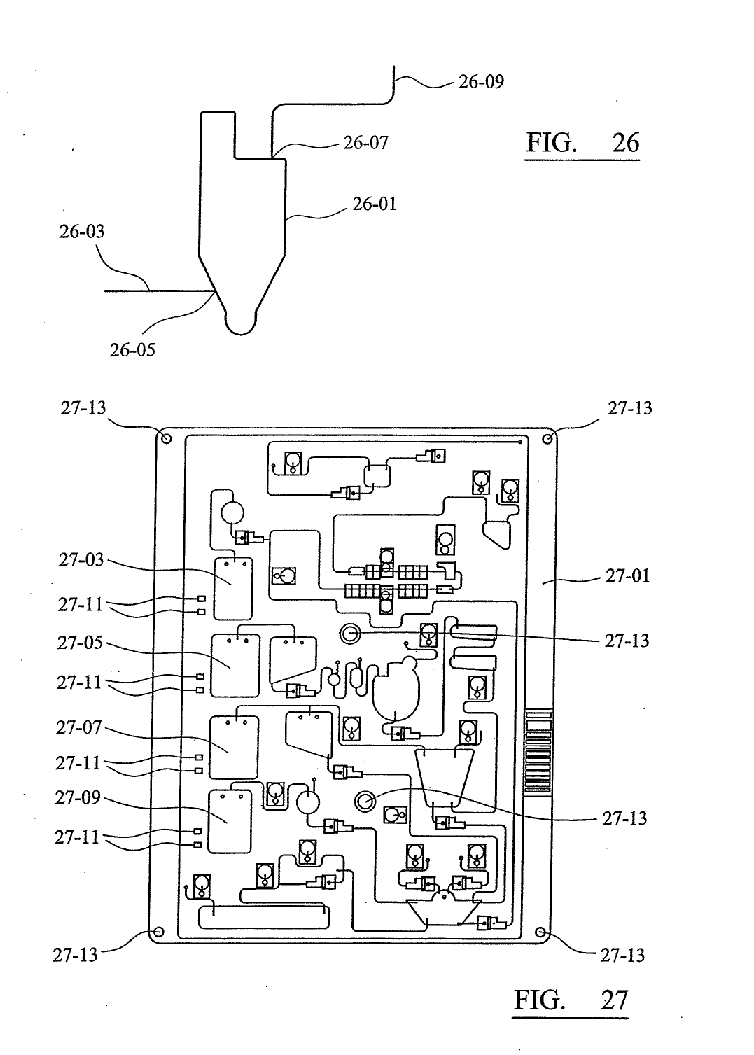

FIG. 24 is an illustration of an arrangement for loading a CE channel

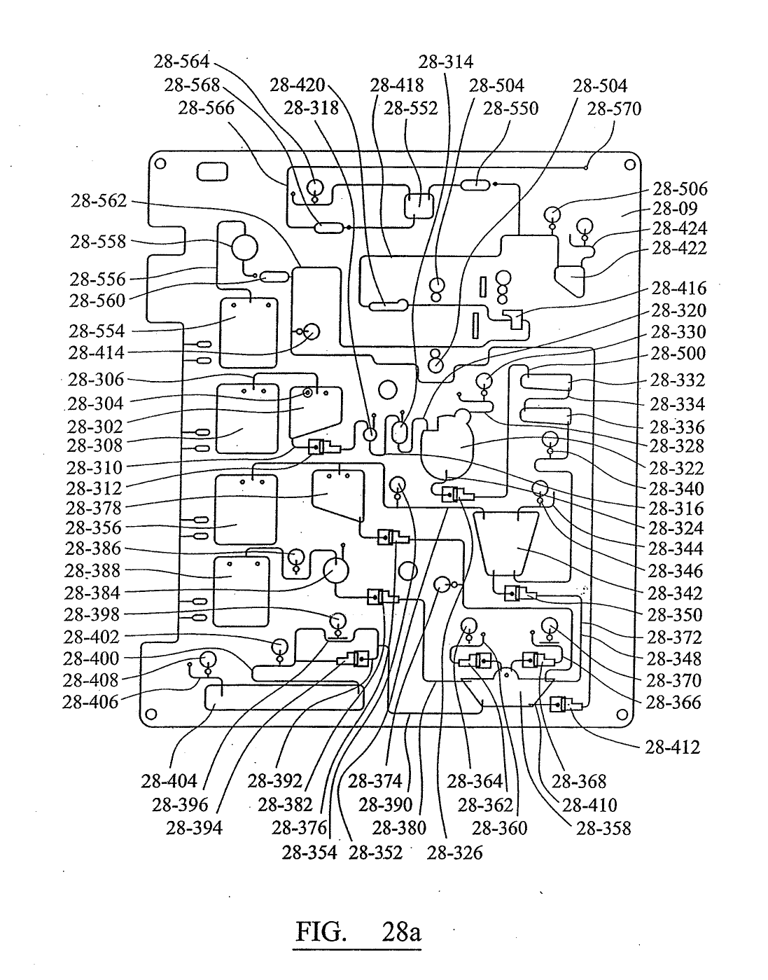

FIG. 25 is an illustration of a further arrangement for loading a CE channel;

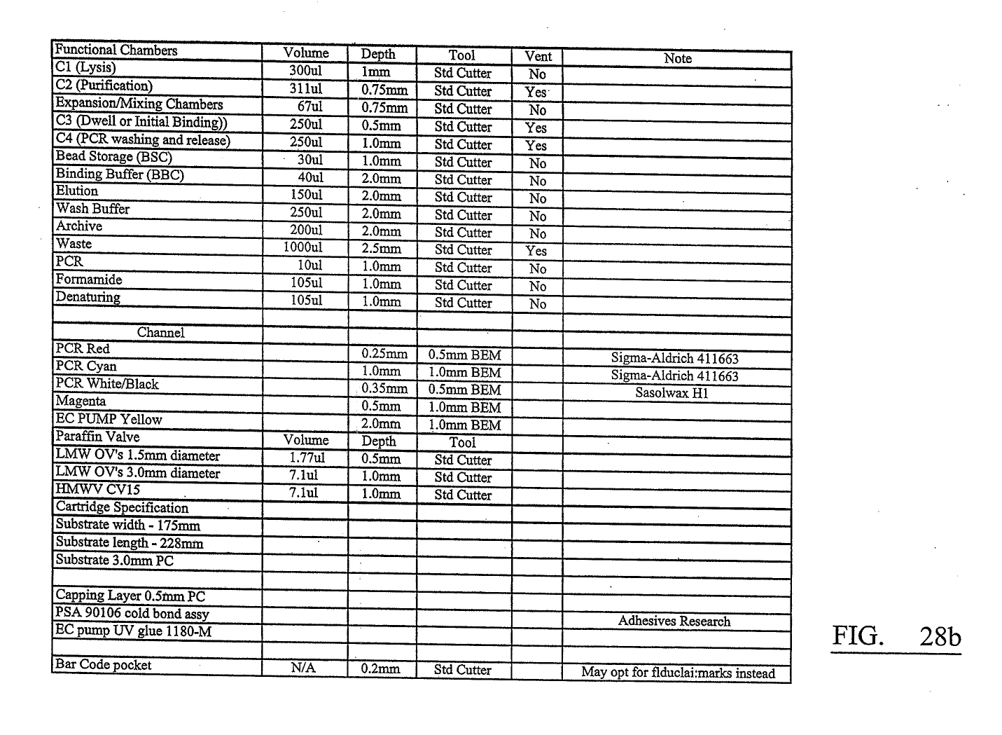

FIG. 26 is an illustration of a further arrangement of a PCR chamber;

FIG. 27 is a front face view of a cartridge;

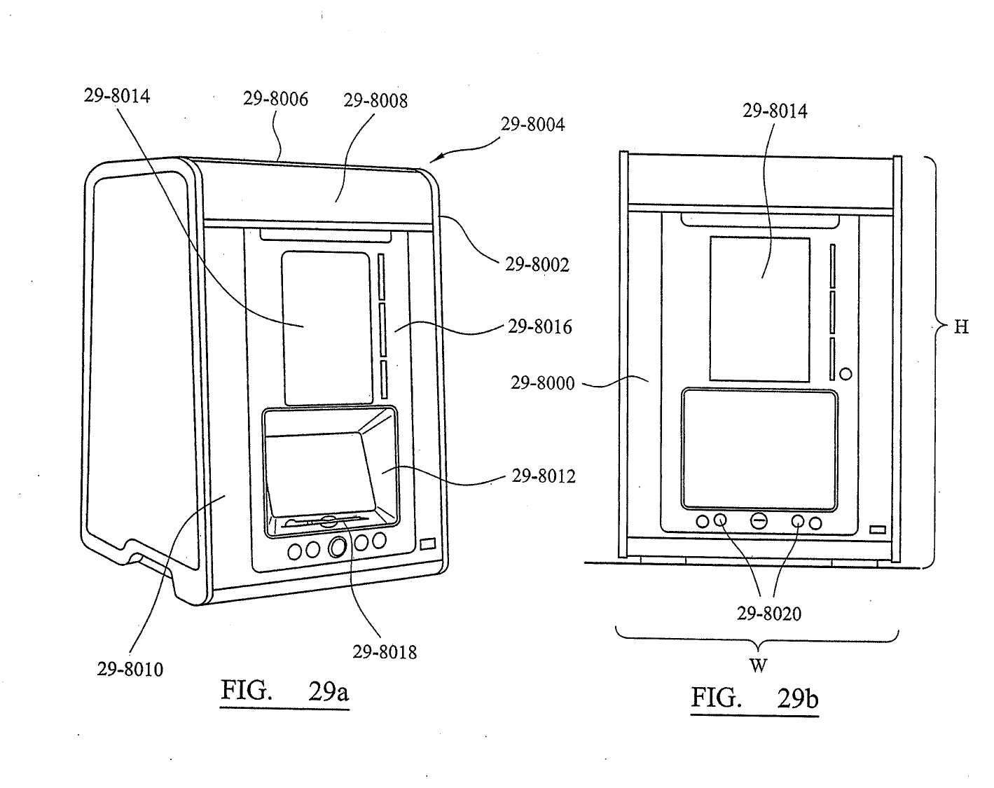

FIG. 28a is a front face view of another cartridge;

FIG. 28b is a table of dimensions and volumes for the FIG. 28a cartridge;

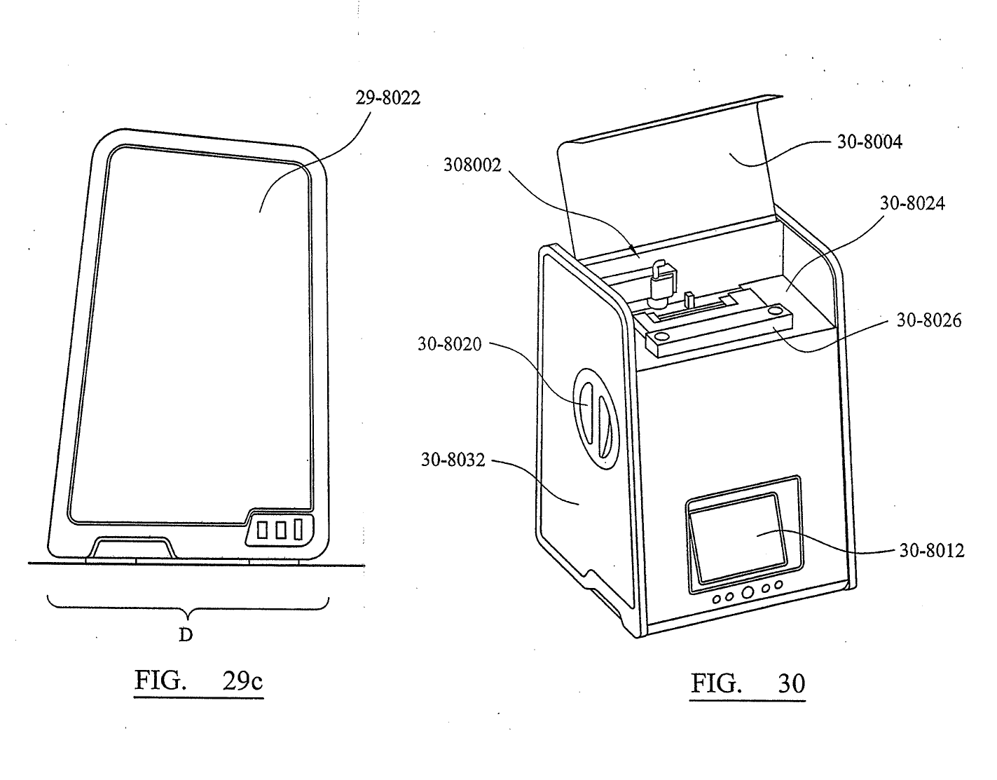

FIG. 29a is a perspective view of an instrument;

FIG. 29b is a front view of the instrument of FIG. 29a;

FIG. 29c is a side view of the instrument of FIG. 29a;

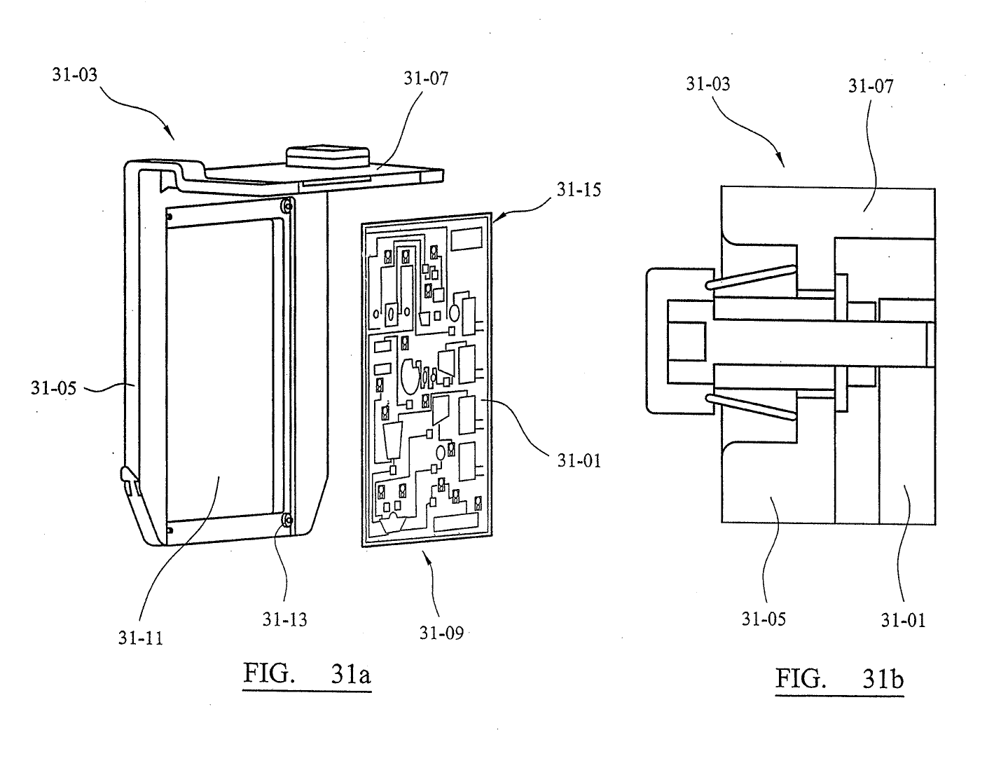

FIG. 30 is a perspective view of another instrument;

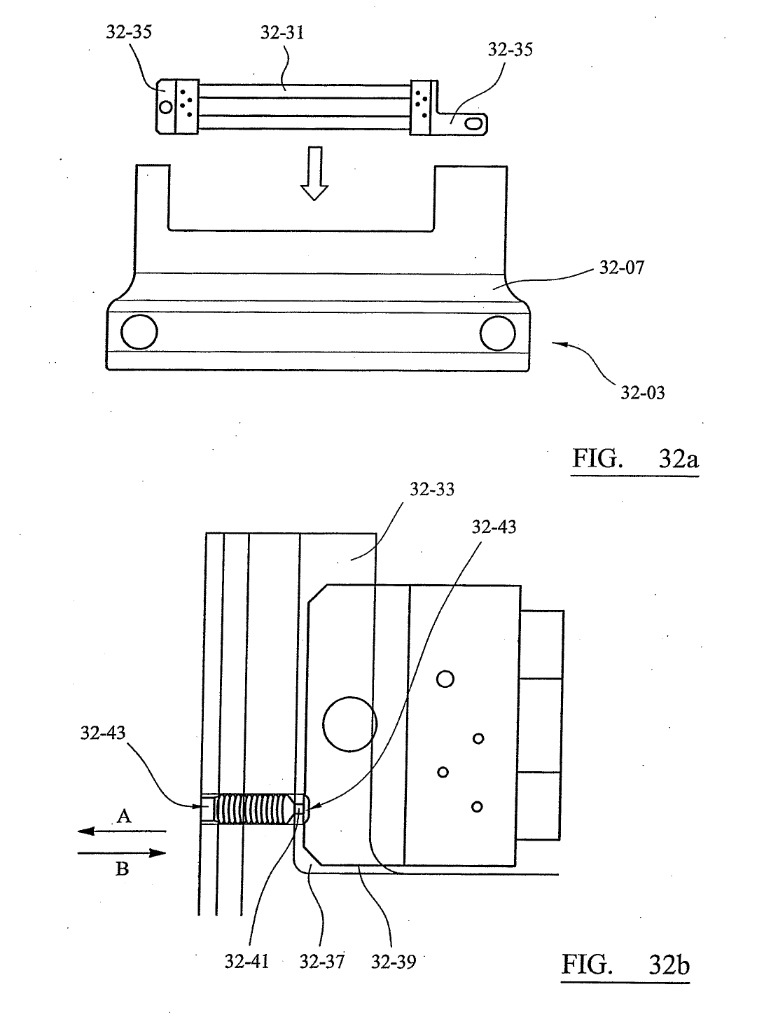

FIG. 31a is an illustration of a carrier, cartridge and CE chip;

FIG. 31b is an illustration of a detail of the carrier to cartridge engagement;

FIG. 32a is an illustration of a carrier to CE chip engagement;

FIG. 32b is a cut away illustration of a part of the FIG. 32a engagement;

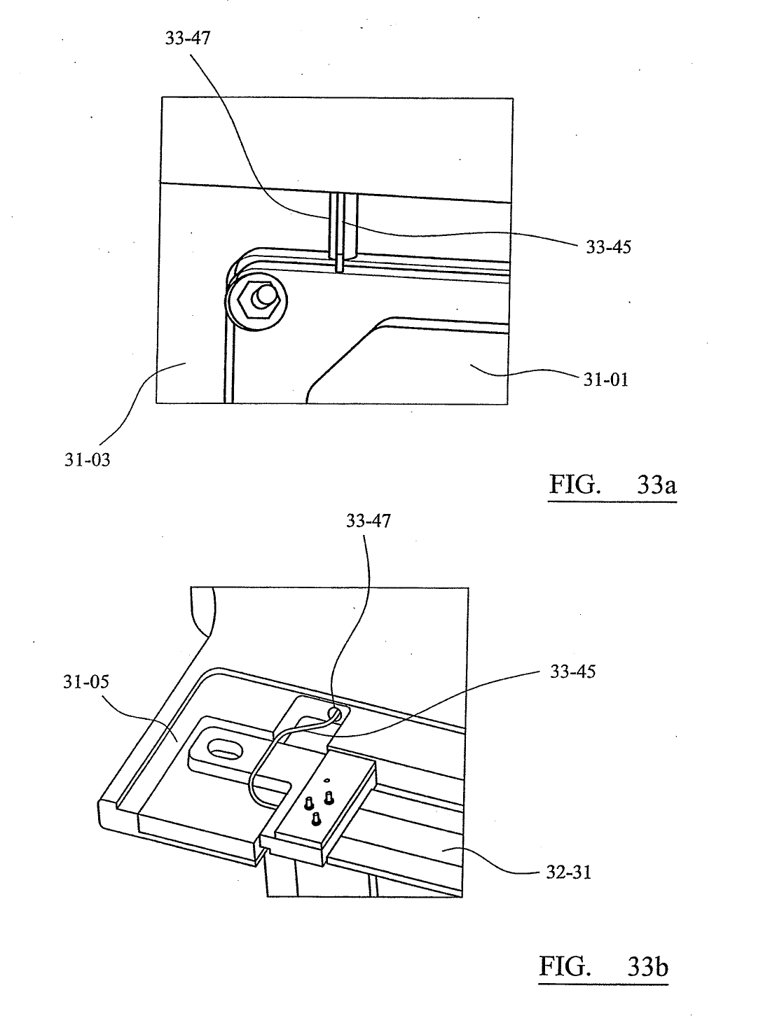

FIG. 33a is an illustration of the tube and cartridge connection;

FIG. 33b is an illustration of the tube to CE chip connection;

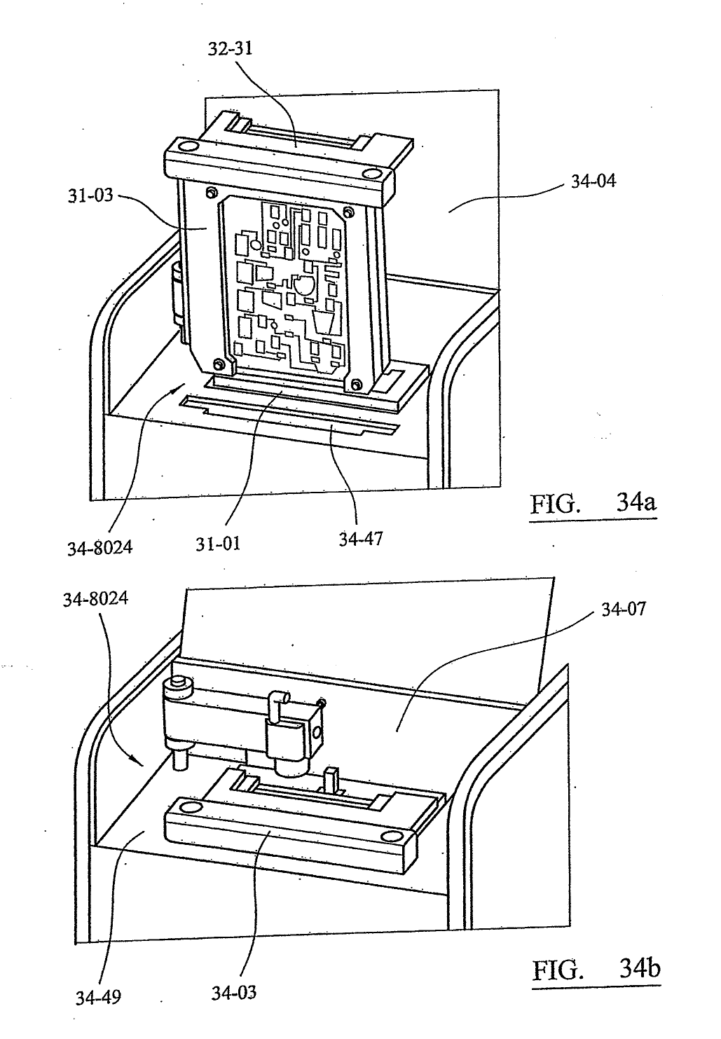

FIG. 34a is an illustration of the carrier being inserted into the instrument;

FIG. 34b is an illustration of the inserted carrier;

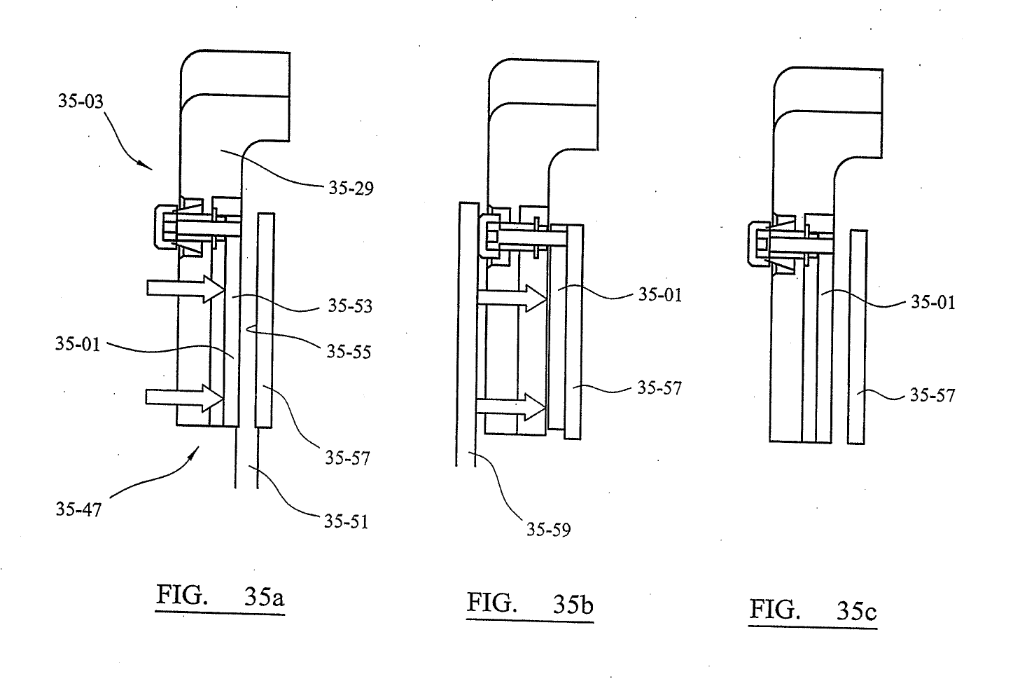

FIG. 35a is an illustration of the cartridge and carrier in the insertion form;

FIG. 35b is an illustration of the cartridge and carrier in the use form;

FIG. 35c is an illustration of the cartridge returned to the carrier;

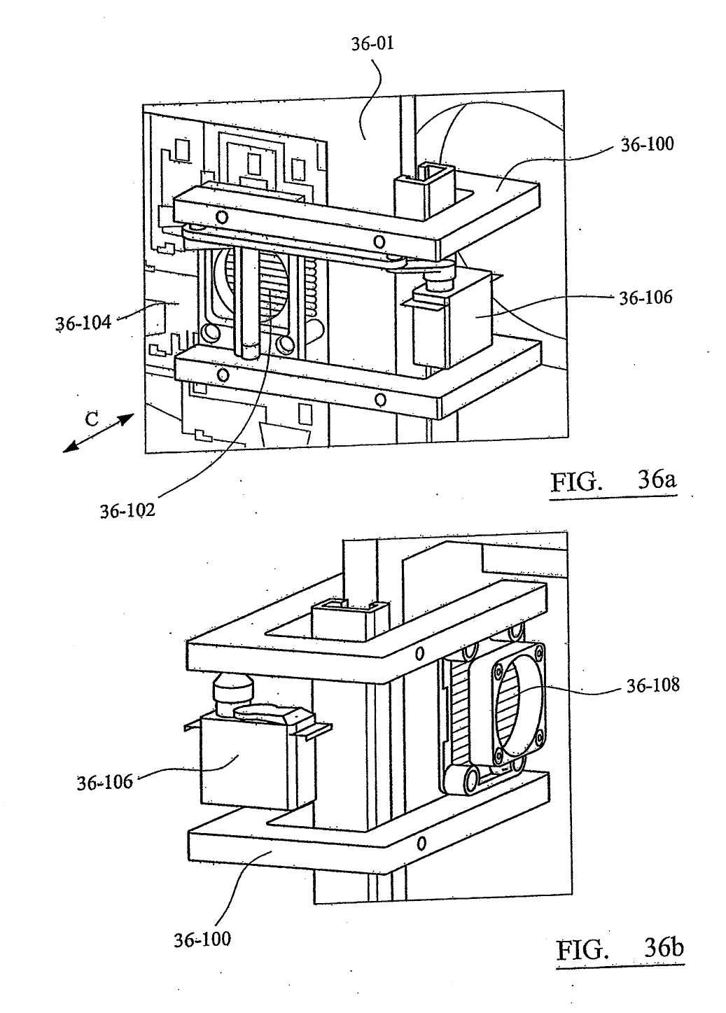

FIG. 36a is a perspective view of the position of the pair of calipers;

FIG. 36b is a perspective view of the back of the pair of calipers;

FIG. 36c is a plan view of the caliper structure in the open form;

FIG. 36d is a plan view of the caliper structure in the closed form;

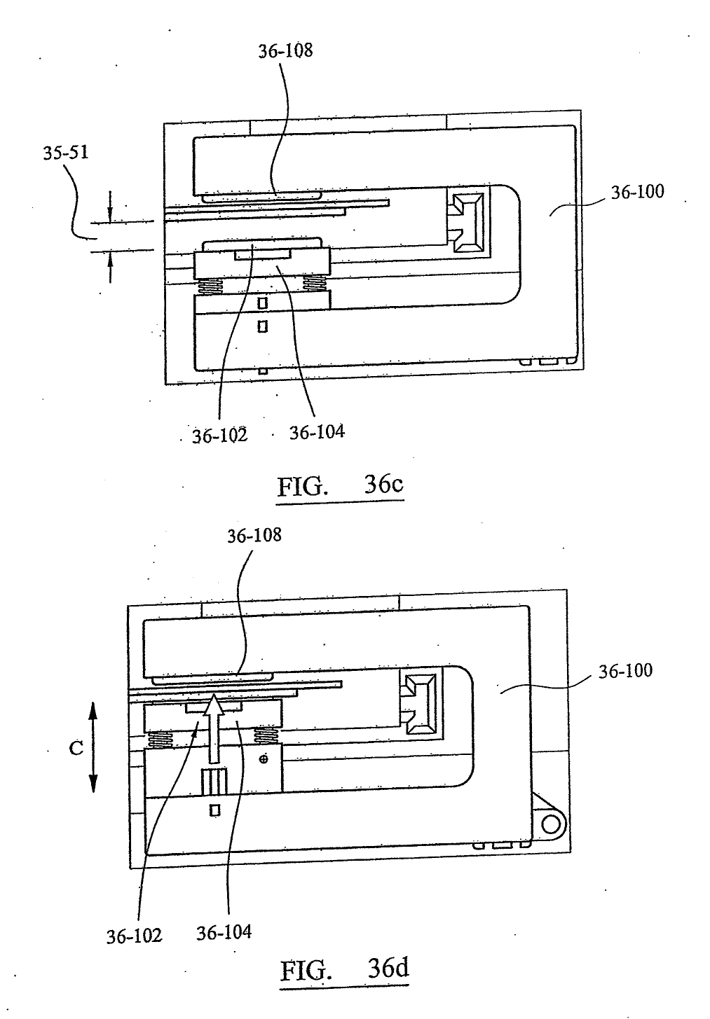

FIG. 37a is a perspective view of the second support of the carrier and CE chip;

FIG. 37b is a partial cut away illustration of the second support and CE chip;

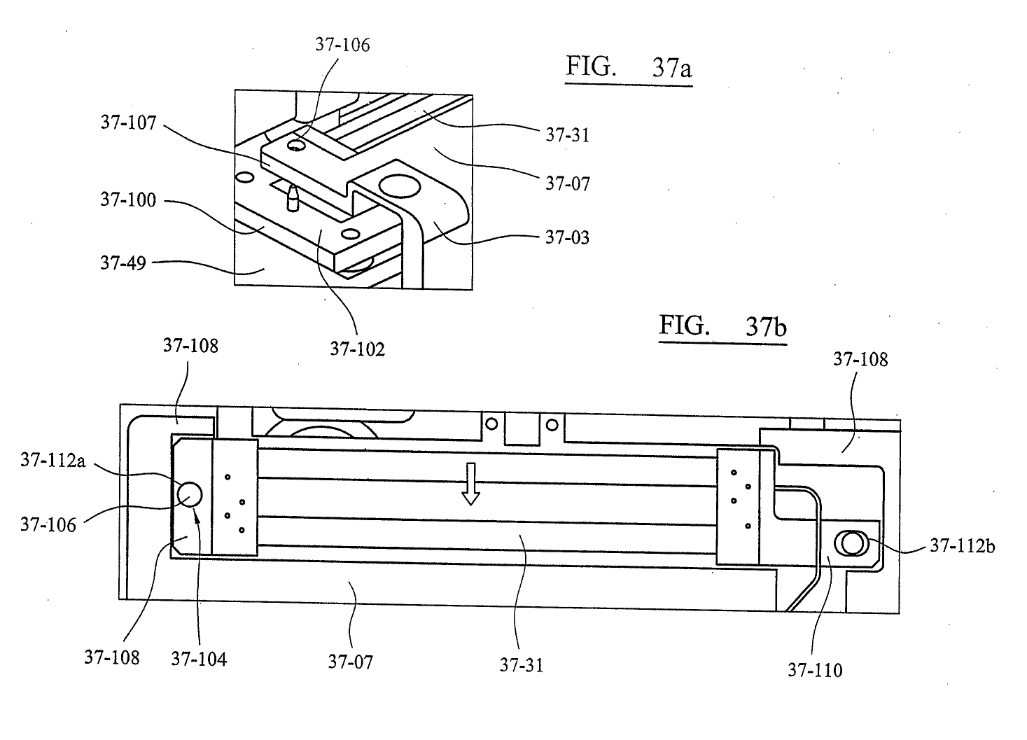

FIG. 38 is a perspective view of the CE chip heater board;

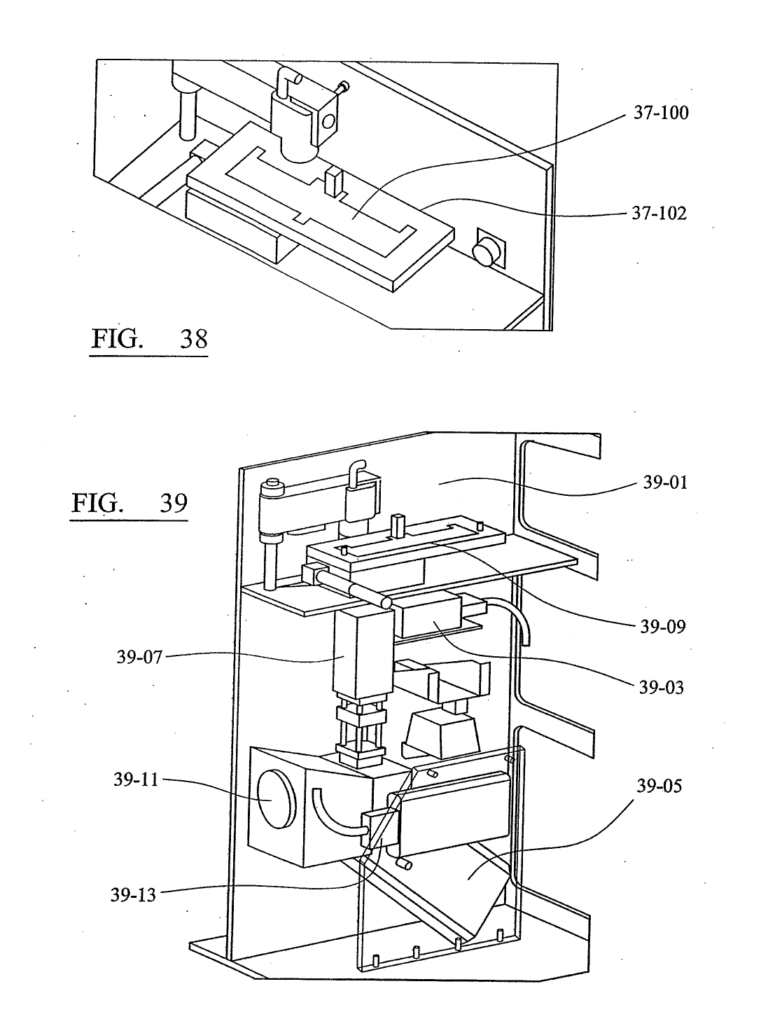

FIG. 39 is a perspective view of an arrangement of the optics;

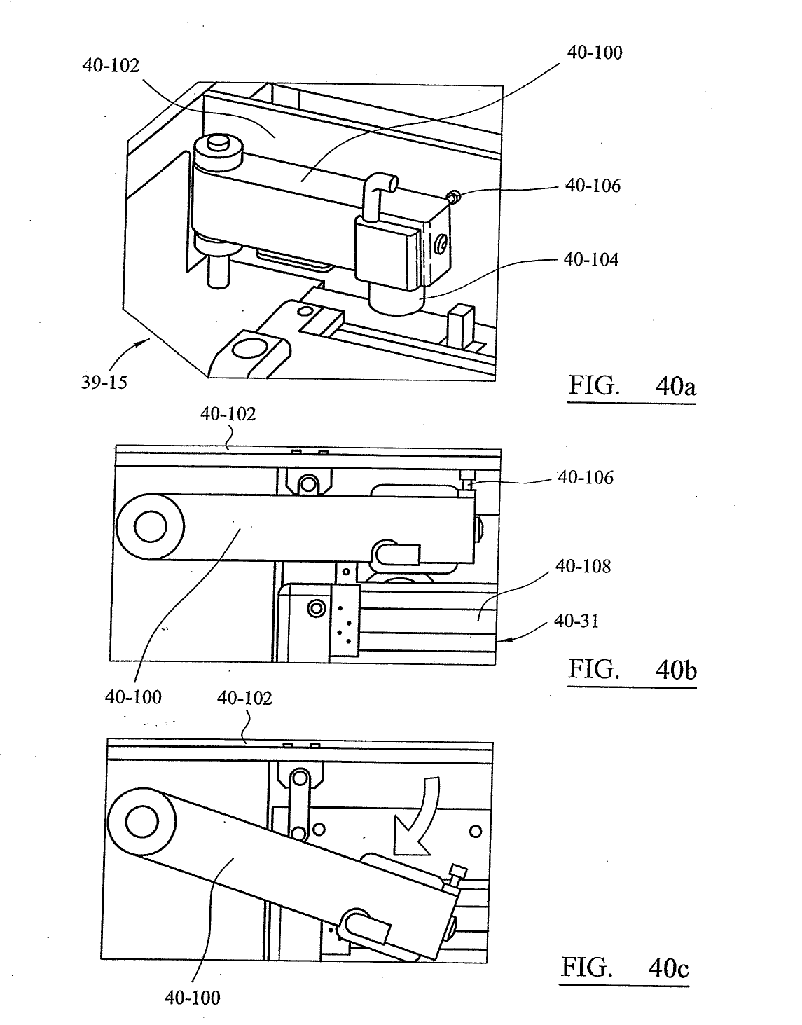

FIG. 40a is a perspective view of the alignment structure;

FIG. 40b shows the alignment structure of FIG. 40a in the stowed position;

FIG. 40c shows the alignment structure of FIG. 40a in the use position;

FIG. 41a shows three positions for an alternative PCR chamber;

FIG. 41b shows two positions for a further PCR chamber;

FIG. 41c shows three positions for a still further PCR chamber;

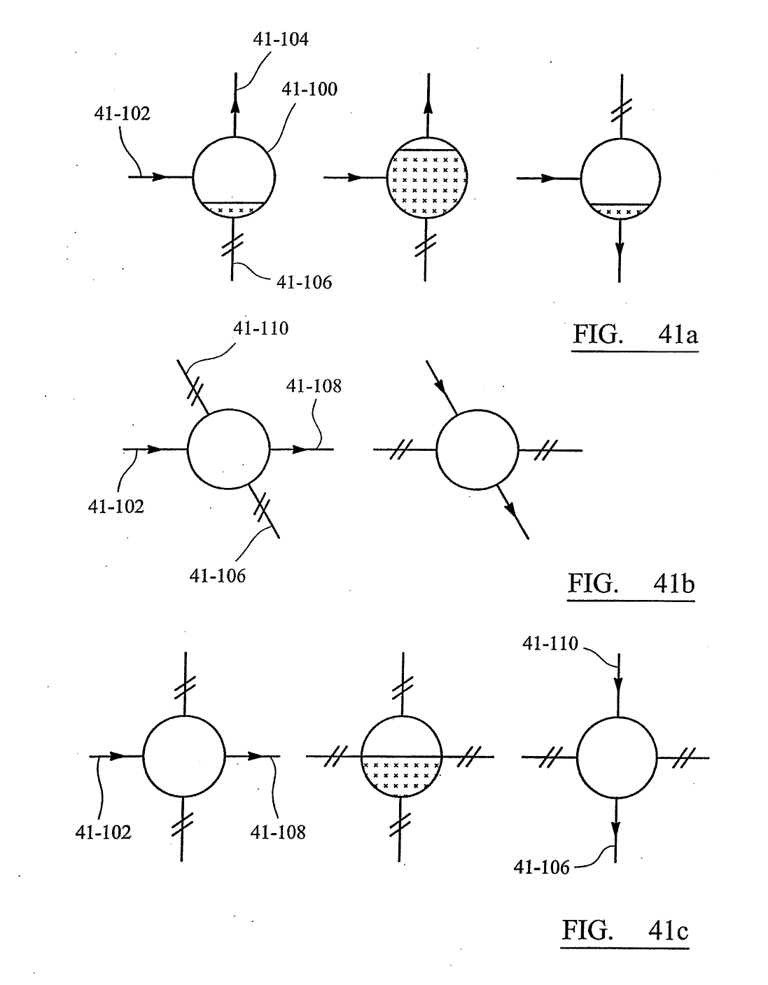

FIG. 42a shows a CE chip;

FIG. 42b shows a detail of the CE chip of FIG. 42a;

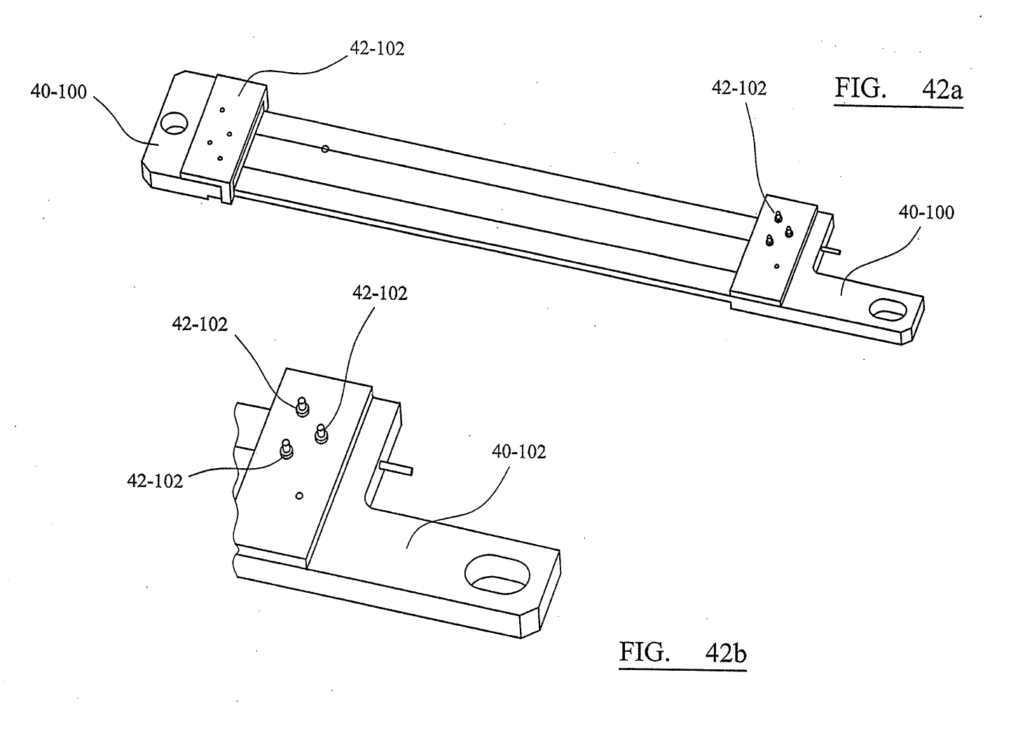

FIG. 43 shows an approach to loading sample to the CE step;

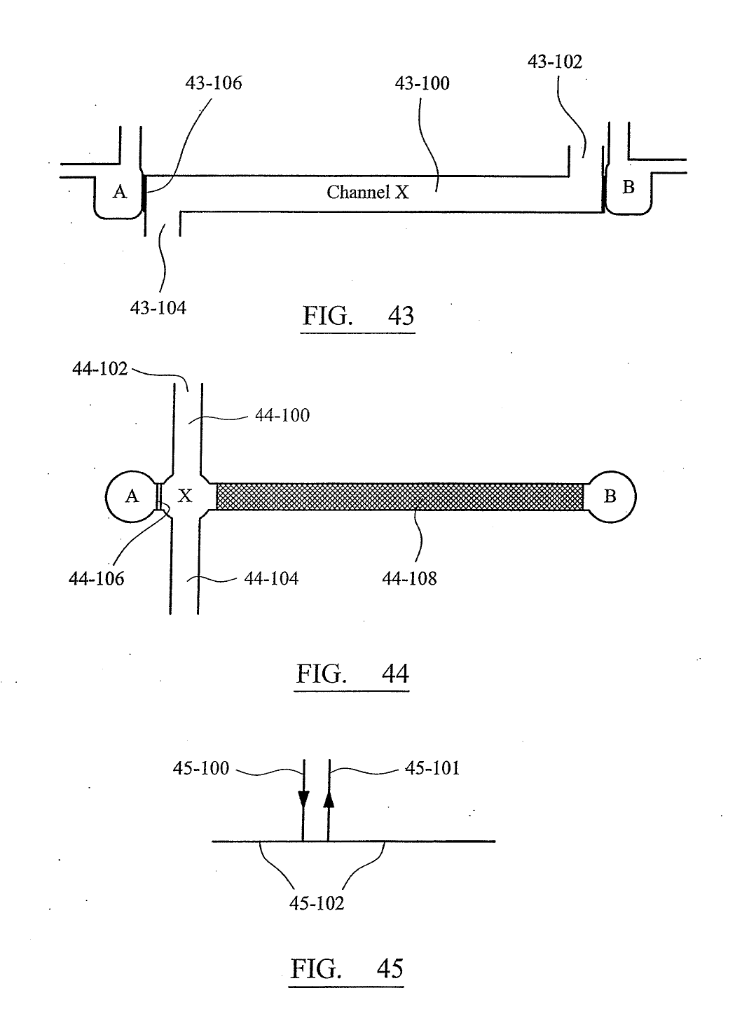

FIG. 44 shows an alternate approach to loading sample to the CE step;

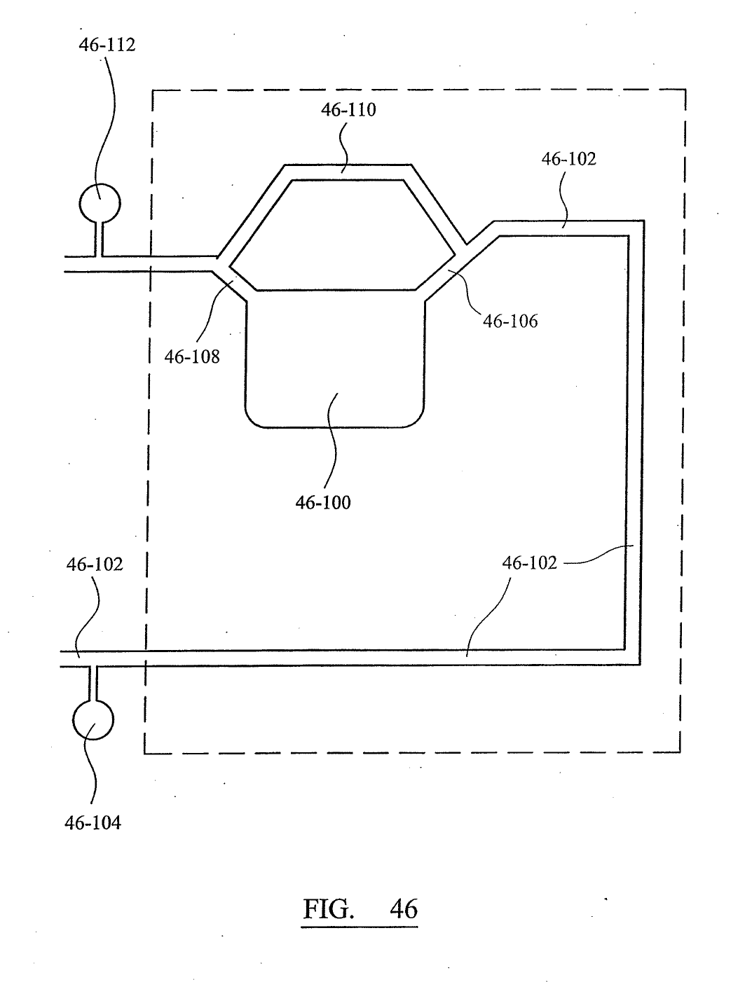

FIG. 45 shows a further alternative for loading sample to the CE step

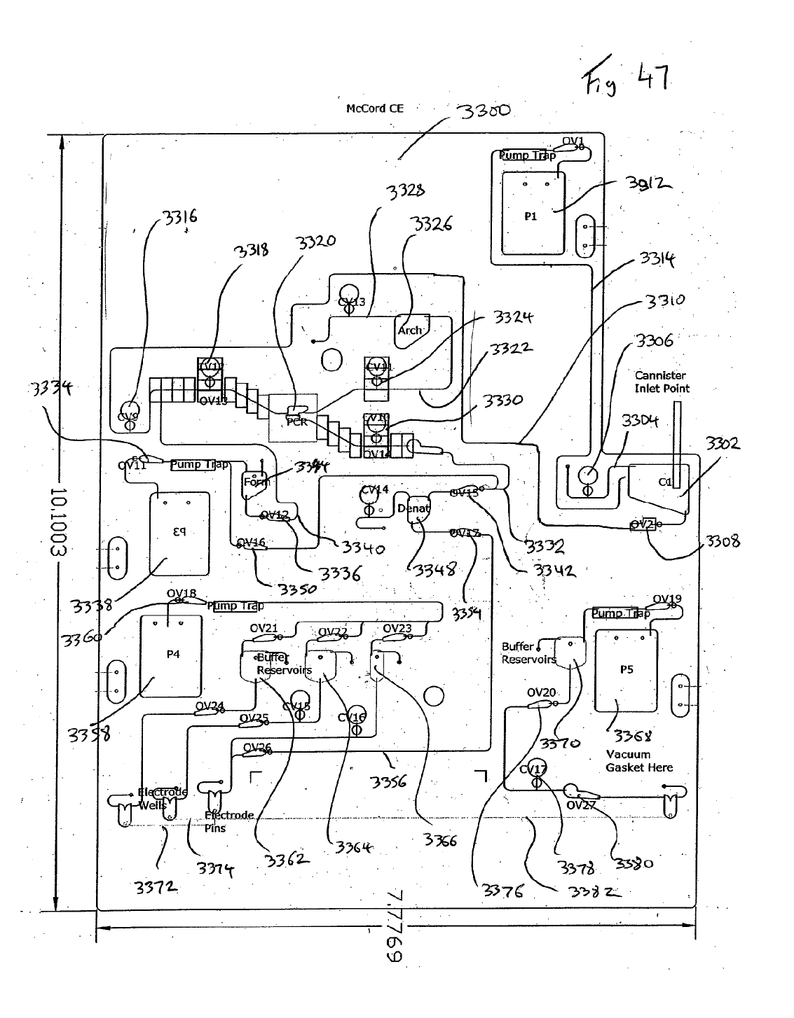

FIG. 46 shows a further PCR chamber; and

FIG. 47 shows a front face view of a cartridge according to an embodiment of the invention.

### BACKGROUND

In a variety of cases it is desirable to be able to analyse a biological sample to obtain information on the sample and/or one or more components of the sample. Such cases include medical diagnostics, for instance to look for disease markers, and forensic science, for instance to establish a DNA profile.

At present, such analyses are conducted by highly trained scientists in a laboratory environment. This means that a significant amount of effort and experience goes into the handling of the samples, the use of the analysis equipment and the formulation of the conclusions reached. However, the need to convey the sample to a laboratory environment and then receive the results back from the laboratory environment introduces a potential time delay between obtaining the sample and obtaining the results thereon. The need to use a laboratory environment and highly trained scientists potentially adds to the time required, as the supply of such people and resources is limited. The need to use a laboratory environment and highly trained scientists potentially adds to the cost as there are capital and running costs associated with such facilities and the scientists.

If fewer laboratory style environments are to be used for the analysis or the staff used are less specialised, then there is the potential for problems with the analysis, unless a proper and reliable system is provided.

The present invention has amongst its potential aims to enable analysis of samples at a greater variety of locations and/or non-laboratory type locations. The present invention has amongst its potential aims to enable analysis by personnel having a lower level of training and/or experience. The present invention has amongst its potential aims to enable lower cost and/or faster analysis of samples. The present invention has amongst its potential aims to enable greater use and/or more successful use of analysis by law enforcement authorities.

Many of the concepts and issues to be addressed by the invention are best understood by way of the following examples. It should be noted, however, that these examples are by their very nature detailed and exhaustive, and that benefits from the present invention arise even when only small sections of the examples are implemented in other embodiments of the present invention.

The various embodiments and examples explain the invention initially in the context of a reference sample; that is a sample collected from a known individual under controlled conditions. An example of a reference sample would be a sample collected by a swab from the buccal cavity of a person who has been arrested, the sample being collected at a police station. The invention is also suited to casework samples; that is a sample collected from a location from an unknown individual under non-controlled conditions. An example would be a spot of blood collected by a swab from a crime scene, with the source of the blood unknown. Where the differences between reference samples and casework samples have an impact on the preferred forms of the instrument, cartridge and methods, the casework sample embodiments are separately described.

The substitution of one or more components by one or more different components or different arrangements of components is also envisaged where particular conditions or issues arise. Again, after the discussion of the reference sample and casework sample contexts for the instrument, these alternatives are described.

As a starting point, it is useful to establish the context of the instrument, cartridge and methods of use in the overall context in which they may be used, by way of example. Thus in FIG. 1 there is a schematic of the overall process into which the present invention fits. This overall process includes a sample 1 which is gathered in a sample collection stage 3. This is followed by a sample preparation stage 5. In the subsequent sample loading stage 7, a prepared cartridge 9 is loaded with the collected and prepared sample 1. The next stage is the cartridge installation stage 15 in which the cartridge 9 is introduced to the instrument 11. The instrument 11 also receives various inputs 13 at the sample loading stage 7 and/or at the cartridge installation stage 15 and/or subsequently.

The structure and processes performed within the instrument 11 and cartridge 9 are described further below.

Once the instrument 11 has completed these stages and achieved the analysis, the next stage is the results stage 17. This is followed by one or more output stages 19, and potential further stages 21 which integrate the analysis into the criminal justice system of that jurisdiction. A wide range of possible links between the various output stages 19 and further stages 21 may be possible, with some being linked to just one stage and others be the result of multiple such stages and/or combinations thereof.

An output stage 19 may include the transmission of the results from the instrument to a remote location for processing. The processing may be performed using complex software and/or hardware tools, before the final results are returned to the instrument 11 or to another computer. Processing the results at a remote location may be preferably in terms of the size, cost or complexity of the software/hardware needed to perform the processing thus only being provided at a limited number of locations, rather than a part of each instrument.

Referring to FIG. 2, the overall process includes a sample receiving step 200, sample preparation step 202, sample amplification step 204, electrophoresis step 206 and analysis step 208 and data communication step 210. To varying degrees these stages may be provided by the instrument or before the sample is provided to the instrument. To varying degrees these stages may be provided by the cartridge used in the instrument or before the sample is provided to the cartridge.

The applicants have submitted various patent applications on instruments, components thereof and methods of use thereof in accordance with such an approach. Because of variations in the sample collection and/or sample preparation stages and/or the form of the sample for the sample loading stage, the applicants have made various modifications to those designs and these are detailed below. In particular, the applicants have made various modifications to reflect the extent to which sample preparation is needed and/or the extent to which sample preparation occurs before or after introduction to the cartridge used by the instrument.

### Variations in Collection, Reception and Preparation

In the US in particular, DNA is often collected in a forensic science context using one of the following approaches.

In one preferred collection approach, the sample is applied to the exposed surface of a matrix. The matrix contains chemicals to promote lysis of the cells within the sample and so release the DNA of interest. The matrix is fibrous in nature and the fibres serve to capture the released DNA within the matrix. The captured DNA is thus retained for transportation and/or storage.

Collection of blood, buccal swab and whole cells is possible in this way. The sample is applied to the surface of the matrix. Room temperature transportation and storage is possible.

When it is necessary to release the DNA from the matrix, the appropriate liquid chemicals are applied. This is normally achieved by taking the matrix with the DNA in it and detaching a small part of the matrix for elution of the DNA. A punch device is commonly used for this purpose. The small part of the matrix is washed with a purification reagent and then rinsed with an eluent (such as 10 mM Tris-HCl, 0.1 mM EDTA, pH 8 as a buffer). The DNA is then available in solution for processing. Further information can be found in U.S. Pat. No. 5,496,562 on the construction and use of such matrices.

Such a matrix based system is available from Whatman Inc, Newton, Mass. 02459-3304, USA as the FTA® card nucleic acid collection approach. Further versions include matrices which change colour on contact with the sample and a matrix provided in a rigid frame.

In the FTA Elute version of the system, a modified chemistry is used in the matrix so as to only require water and heat to give the eluted DNA. The small piece of the matrix is punched into a tube, contacted with water, centrifuged to allow excess water to be removed, contacted with further water at 95° C. for 30 minutes and then centrifuged to give the elute DNA sample.

In the EasiCollect® device, the matrix is provided in a rigid casing with an aperture there in which leads to the matrix. The surface of the matrix is protected by a protective layer. Extending from the casing is a stem with a swab at the end. The swab is used to collect the sample. The protective layer is removed from the matrix and the stem is bent at a predetermined location to bring the swab, and hence sample, into contact with the matrix. The matrix acts in the manner described above to lyse and store the sample.

Other collection approaches include the GenePlate® collection system available from GeneVault of 6190 Corte Del Cedro, Carlsbad, Calif. 92011, USA and the complimentary elution chemistry offered as the GeneSolve® kit. This kit is also described as applicable to eluting from FTA matrices and Guthrie card matrices. The small piece of the matrix is punched into a tube and then a mixture of a lycophilized reagent and protease is added. An incubator/shaker is used to agitate tube at 65° C. for an hour. This is then centrifuged and excess fluid is removed. A further reagent is then added before further centrifuging and prepared sample removal from the tube. A purification stage may then be applied using Qiagen's QIAmp DNA Blood Mini kit. The DNA present may be quantified before further processing.

In a further preferred collection approach, a plastic holder is employed which includes a handle for the operator and label for writing on. A slideably cover provided on the holder can be drawn back to expose the cotton paper sample collection area. That area is brought into contact with the sample, for instance to take a buccal sample. The cover is then slid back into position to protect the sample against contamination. The paper acts in a similar manner to that outlined above and can be transported and stored at room temperature.

To obtain the DNA for analysis, the paper is exposed at a small piece of the matrix detached, for instance by punching. The small piece of the matrix is then contacted with a preparation solution and incubated (for instance 70° C. for 20 minutes) to place the DNA in a state suitable for PCR.

A system of this type is detailed in WO02/096480. A system of this type is available from The Bode Technology Group Inc, 10430 Furnace Road, Suite 107, Lorton, Va. 22079, USA and includes the Bucall DNA Collector, PunchPrep Solution reagents and Promega's PowerPlex® 16 HS reaction mix.

### Impact on the Process Stages

In contrast with some previous embodiments presented by the applicants, the sample receiving step 200 and/or sample preparation step 202 may be provided outside of the cartridge 9 and/or instrument 11.

Thus, the sample 1 is received on the initial collection device and in the sample preparation step 202, an part of the sample collection device is taken and the key components within the sample are contacted with the reagents and/or components intended to prepare the sample for the subsequent steps. In this embodiment, the sample preparation step 202 contacts the sample with reagents to elute the DNA from the matrix which forms the part of the sample collection device. This may involve any of the preparation, elution or purification steps described above for the approaches.

Once the sample preparation step 202 has been completed, the sample may be introduced to the cartridge 9 and/or instrument 11. The sample can then pass straight to the sample amplification step 204 within the cartridge. In the sample amplification step 204, the DNA is contacted with amplification reagents and provided with the conditions necessary to achieve amplification through PCR.

In the electrophoresis step 206, the amplified DNA is conveyed to a start point for a mobility based separation within a capillary. An electric field is then used to separate the complex DNA amplicons into different size clusters.

In the analysis step 208, the channel is inspected to establish the relative position and hence size of elements detected in the capillary. This is achieved by an excitation light source, fluorescent markers associated with the elements to be detected and suitable optics to detect the fluorescent light resulting.

In the data communication step 210, the instrument compiles the necessary data packet for transmission and transmits it to a remote location for consideration. The data packet includes information on the electrophoresis results, sample identity and other information. The analysed results may be received by the instrument as part of the data communication step 210.

Some data processing may be performed on the instrument itself, for instance to deconvolute the analysis results to indicate the peaks indicative of alleles present.

The instrument can be provided in a format which considers a single sample at a time, or can be provided in a format which considers multiple samples at a time. The multiple samples may each be run on separate cartridges, but modified cartridges which handle multiple samples are possible. The handling of multiple cartridges is beneficial in allowing a single set of controllers, power supplies, optics and the like to consider multiple samples, with reduced capital costs.

### Off Cartridge Steps

In the above mentioned collection approaches, the DNA is present on a solid matrix in bound form. As a result, the DNA needs to be released before it can be introduced into a micro-fluidic part of a cartridge for processing.

To achieve this release, a sample preparation container is used. The container can be opened to receive the matrix. In practice, this is likely to be only a small piece of the matrix which is detached from the remaining matrix. The remaining matrix may be stored to provide a reference of the sample. The piece may be detached by cutting, punching or other such approaches. The piece is placed in the container where the necessary processing and/or conditions to achieve release of the DNA into a liquid phase are provided.

The processing and/or conditions may take the form of exposing the matrix, detaching a small piece there from and placing that in an open container. The container may already contain the reagents to release the DNA or those may be added. Once present, the container may be sealed. The processed contents may be dispensed from the container into the cartridge, for instance into a sample receiving chamber.

The structure and operation of various cartridges are set out below.

The processing and/or conditions may alternatively take the form of exposing the matrix, detaching a small part there from and placing that in an open container. The container may already contain the reagents to release the DNA or those may be added. Once present, the container may be sealed. The container and contents may be subjected to centrifuging to provide an at least partial separation of fluid from the DNA. The container may be opened or liquid may otherwise be removed. The container may be opened or further reagents may otherwise be provided. The container and contents may then be heated, for instance incubated, for a period of time. This may be done by a section of the instrument or in a separate device. The container and contents may be subjected to further centrifuging to provide an at least partial separation of fluid from the DNA. The container may be opened or liquid may otherwise be removed. The processed contents may be dispensed from the container into the cartridge, for instance into a sample receiving chamber.

The processing and/or conditions may alternatively take the form of exposing the matrix, detaching a small part there from and placing that in an open container. The container may already contain the reagents to release the DNA or those may be added. Once present, the container may be sealed. The container and contents may then be heated, for instance incubated, for a period of time. This may be done by a section of the instrument or in a separate device. The container and contents may be subjected to centrifuging to provide an at least partial separation of fluid from the DNA. The container may be opened or liquid may otherwise be removed. The container may be opened or further reagents may otherwise be provided. The container and contents may be subjected to further centrifuging to provide an at least partial separation of fluid from the DNA. The container may be opened or liquid may otherwise be removed. The processed contents may be dispensed from the container into the cartridge, for instance into a sample receiving chamber.

The processing and/or conditions may alternatively take the form of exposing the matrix, detaching a small part there from and placing that in an open container. The container may already contain the reagents to release the DNA or those may be added. Once present, the container may be sealed. The container and contents may then be heated, for instance incubated, for a period of time. This may be done by a section of the instrument or in a separate device. The processed contents may be dispensed from the container into the cartridge, for instance into a sample receiving chamber.

In any of these processes, it is possible to apply one or more processes and/or one or more reagents to provide for purification of the sample and/or the DNA contained therein.

With the matrix retained in the container and the DNA, or other relevant part of the sample, dispensed into the cartridge, the cartridge's operation comes into play.

### Cartridge

Key to the operation of the instrument is a disposable, single use cartridge 9. This cartridge 9 is intended to only process and provide the results for analysis on a single occasion. The disposable nature of the cartridge 9 places a number of constraints on the cartridge 9 in terms of the materials which can be used, because of the need to keep manufacturing, assembly or purchase costs low.

The detailed layout of some possible cartridges are now described, together with some general concepts which reflect the possible variations in the locations at which sample preparation is conducted.

In FIG. 47 the cartridge 3300 is provided with a sample introduction chamber 3302 connected to a channel 3304 leading past open valve 3306 to the outside of the cartridge 3300 so as to allow venting as the sample enters the cartridge 3300.

Once the sample is loaded, the valve 3306 is closed and valve 3308 in channel 3310 is opened. Electrochemical pump 3312 is activated to drive fluid along channel 3314 into the sample introduction chamber 3302 and hence drive the sample along channel 3310.

The channel 3310 leads past open valve 3316 and open valve 3318 to the PCR chamber 3320.

The outlet channel 3322 from the PCR chamber 3320 leads past open valve 3324 and into archive chamber 3326. The archive chamber 3326 is vented through vent channel 3328 to allow filing of the PCR chamber 3320.

Once present in PCR chamber 3320, valves 3318 and 3324 are closed and together with closed valve 3330 in outlet channel 3332 serve to isolate the PCR chamber 3320 from the rest of the cartridge during PCR.

Provided within the PCR chamber 3320 is a bead loaded with the reagents, a multimix, needed for the PCR process. The reagents/multimix include primers dNTPs and PCR reaction mix, including Tris buffer, MgCl₂, NaCl and BSA. These reagents are released into the sample once it contacts the bead in the PCR chamber 3320 and the temperature is raised above ambient temperature.

The above circuit overall, is sufficient to receive and perform PCR on the sample (as well as storing an archive of the PCR product). The sample preparation is conducted, if necessary, outside of the cartridge.

Subsequently, the further components shown in FIG. 47 can be used to prepare, denaturation step, and transfer the now amplified DNA from the PCR chamber 3320 into the electrophoresis step 206.

By opening valve 3334 and valve 3336 fluid can be driven from second electrochemical pump 3338 along channel 3340 past valve 3318 and into the PCR chamber 3320. This can initially dispense a sample of the PCR product to the archive chamber 3326.

Leading from the PCR chamber 3320 is outlet channel 3332. By opening valve 3342, with valve 3324 closed, the second electrochemical pump 3338 can be used to drive the amplified product through chamber 3344 and past valve 3336 to a denaturation chamber 3348. Formamide in chamber 3344 is driven into the denaturation chamber 3348 from denaturing reagent storage chamber 3344 as a result, in combination with the size standards to be used in the capillary electrophoresis step 206.

The amplified material is held in the denaturation chamber 3348 for the necessary time and at the necessary temperature to complete the denaturing process. Once this has been achieved, the sample is pumped by the second electrochemical pump 3338 into the electrophoresis step inlet 3352 by the passage of fluid along channel 3349 past valve 3350, now open valve 3354 and channel 3356.

Third electrochemical pump 3358, valve 3360, buffer chambers 3362, 3364 and chamber 3366, in combination with various valves control the sequence in which agents are provided to the electrophoresis step 206 in the form of capillaries 3372 and 3374.

Fourth electrochemical pump 3368 is used to drive fluid through buffer reservoir chamber 3376 and valves 3378 and 3380 to the capillary 3380 as required.

Throughout the operations described above and in the sections that follow, various checks are made on operating conditions, component performance and successful operation so as to ensure the processing is correctly provided from start to finish. Errors or problems are indicated to the operator.

The above operation is in contrast with the sample preparation and normalisation in cartridge approach provided for in FIG. 3a and elsewhere. The sample preparation in this case is provided off cartridge.

### Features Useful to the Invention

To assist with the understanding and operation of the instrument, cartridge, components and methods in their revised form, details of the instrument, cartridge, components and methods are provided in a more general sense.

FIG. 3a is an illustration of that part of the sample receiving step 200 provided on the cartridge 9, the whole sample preparation step 202 and the whole sample amplification step 204. The subsequent steps and their respective pasts of the cartridge 9 are illustrated separately.

FIG. 3b provides details of the volumes of the various chambers used, the depths (into the page in effect) for the various components and the overall dimensions of this part of the cartridge 9.

The cartridge 9 is provided with a sample introduction chamber 302 connected to a channel 304 leading to the outside of the cartridge 300. This forms those parts of the sample receiving step 200 provided on the cartridge 9.

The sample preparation step 204 follows. To provide this, the sample introduction chamber 302 is connected to a pumping fluid channel 306 and hence to a first electrochemical pump 308. The sample introduction chamber 302 has an outlet channel 310 which passes valve 312 and provides an inlet to purification buffer chamber 314. Valve 312 is initially open.

Purification buffer chamber 314 is connected via channel 316 to bead storage chamber 318. The bead storage chamber 318 is connected via channel 320 to initial mixing chamber 322. The outlet channel 324 from initial mixing chamber 322 is blocked by closed valve 326, but a vent channel 328 is open because valve 330 is open initially.

The outlet channel 324 leads past valve 326 to a first further mixing chamber 332 and then through channel 334 to second further mixing chamber 336. The outlet 338 from the second further mixing chamber 336 leads past valve 340 to incubation chamber 342, where bubble mixing assists the DNA to bead binding process.

The incubation chamber 342 has a vent channel 344 provided with valve 346 and an outlet channel 348 which is initially closed by valve 350. The incubation chamber 342 is also provided with a pumping fluid inlet channel 352 which passes valve 354 and is connected to second electrochemical pump 356.

The outlet channel 348 from the incubation chamber 342 leads to capture chamber 358 where the beads and hence bound DNA are collected. The capture chamber 358 is provided with a first vent channel 360 which passes first valve 362 and second valve 364. The capture chamber 358 is also provided with a second vent channel 366 which passes first valve 368 and second valve 370.

Also connected to capture chamber 358 is wash buffer channel 372. The wash buffer channel is connected to first valve 374 and second valve 376 and leads from second electrochemical pump 356 through wash buffer chamber 378 to the capture chamber 358.

Also connected to capture chamber 358 is an elution liquid channel 380. The elution liquid channel 380 is connected to first valve 382, elution liquid storage chamber 384, second valve 386 and back to third electrochemical pump 388.

The capture chamber 358 has a wash outlet channel 390 which splits into a first wash outlet channel section 392 which passes valve 394, and into a second wash outlet channel section 396 which passes valve 398. After passing their respective valves 394, 398, the first wash outlet channel section 392 and second wash outlet channel section 396 rejoin one another to form further wash channel 400. The further wash channel 400 leads past valve 402 into waste chamber 404. The waste chamber 404 is vented along vent channel 406 past valve 408. These elements provide the sample preparation step 202.

To provide the sample amplification step 204, capture chamber 358 is also provided with elution outlet channel 410 which leads past valve 412 and past valve 414 and into PCR chamber 416. The outlet channel 418 from the PCR chamber 416 leads past valve 420 into archive chamber 422. The archive chamber 422 is vented through vent channel 424. The role of the archive chamber 422 is described further below.

Provided within the PCR chamber 416 is a bead loaded with the reagents, a multimix, needed for the PCR process. The reagents/multimix include primers dNTPs and PCR reaction mix, including Tris buffer, MgCl₂, NaCl and BSA. These reagents are released into the sample once it contacts the bead in the PCR chamber 416 and the temperature is raised above ambient temperature.

The above circuit overall, is sufficient to receive, retain, wash, elute and perform PCR on the sample, as well as storing the waste from the process and an archive of the PCR product.

Subsequently, the arrangement shown in FIG. 4 can be used to transfer the now amplified DNA from the PCR chamber 416 into the electrophoresis step 206.

In FIG. 4, the PCR chamber 416 is the same PCR chamber 416 which was illustrated in FIG. 3 and described above. Other features were omitted from FIG. 3 to improve the clarity of that Figure.

Leading from the PCR chamber 416 is a denaturing feed channel 500 which is connected to an amplified material mixing chamber 502. The amplified material is pumped from PCR chamber 416 by the action of fourth electrochemical pump 504 which is connected to channel 506, hence to denaturing reagent storage chamber 508 and through channel 510 to the PCR chamber 416. Formamide is provided in the denaturing reagent storage chamber in the preferred form.

These components are isolated from the PCR chamber 416 during the sample amplification step 204 by closed valve 512 and closed valve 514. Both valve 512 and 514 are opened and valves 516 and 518 are closed to convey the amplified material away from the PCR chamber 416.

From the denaturing feed channel 500, the amplified material and denaturing reagents enter the first amplified material mixing chamber 502, pass through channel 520, into second amplified material mixing chamber 522, through channel 524 and into third amplified material mixing chamber 526. Whilst the third amplified material mixing chamber 526 fills, valve 528 is shut and vent 530 is open. An overall volume of 45 μl is provided, 5 μl from the PCR chamber and 40 μl from the denaturing reagent storage chamber 508.

The amplified material is held in the third mixing chamber 526 for the necessary time and at the necessary temperature to complete the denaturing process. Once this has been achieved, the valve 528 is opened and further pumping by the fourth electrochemical pump 504 pumps the denatured material to the electrophoresis step inlet 532. At the inlet 532, the denatured material passes out of the plane of the cartridge 9 and to the electrophoresis cartridge section behind. Once past through the inlet 532, valve 534 is shut to isolate the cartridge 9 from the electrophoresis cartridge section 600.

The overall result of this structure is the pumping of the amplified DNA to a start point for the electrophoresis step 206.

The transfer from PCR to CE steps is provided in a way which allows easy integration of the steps, does not impact upon the temperature and pressure stability required in PCR and achieves minimal sample loss during transfer. Automated mixing of the sample and size standards during transfer and possibilities for pre-concentrating the sample before CE are also rendered possible.

The overall configuration of the electrophoresis step 206 can be seen in the side view of FIG. 5a and front view of FIG. 5b.

The inlet 532 leads from the plane of the cartridge 9, through into the plane of the electrophoresis cartridge section 600. Here, the inlet 532 leads into the top section 602 of an electrophoresis feed reservoir 604. The top section 602 is empty, but the lower section 606 is provided with the gel 608 which also fills the capillary 610. The sample is pumped into the electrophoresis feed reservoir 604 by a fourth electrochemical pump, not shown.

Sample flow from the reservoir 604 into the correct position within the capillary 610 is achieved using electrophoresis as the transport mechanism.

In this embodiment, the injector structure provided within the capillary cartridge section 600 is a double T injector. This includes a first electrode location 612, second electrode location 614 provided at the other end of the long capillary 616 in which the size based separation is achieved. A third electrode location 618 and fourth electrode location 620 are provided in side arms 622 and 624 respectively. The side arms are offset relative to one another, with side arm 624 further towards the second electrode location 614, than the side arm 622.

Initially, sample is drawn from the liquid phase in the reservoir 604 through the interface with the gel provided in the reservoir 604 and hence into the gel by a voltage applied to the electrode present at the third electrode location 618. Once the sample has been drawn past the fourth electrode location 620, a voltage is also applied to the electrode at the fourth electrode location. Generally, the electrode at the third electrode location may be at a voltage of 600V and the electrode at the fourth electrode location may be at a voltage of 200V. The voltage may be floating for the electrodes at the first 612 and second 614 electrode locations.

This situation results in sample being drawn along side arm 624, along the section 626 and into side arm 622, such that sample is present in the two side arms 622 and 624 and the section 626 of the capillary 616.

This gives the plug of sample upon which the electrophoresis's to act in the section 626.

To reduce the cost of the electrodes used, consistent with the cartridge being single use, platinum coated, gold coated, carbon, nickel and other lower cost electrodes may be used.

Once positioned, the separation voltages are applied: 1500V at the electrode at the second electrode location 614; 0V at the electrode at the first electrode location 612; and 200V at the electrodes present at the third electrode position 618 and fourth electrode positions 620.

The capillary 616 is filled with a gel matrix which preferentially retards the speed of progress of elements within the DNA as their size increases. The result is a size based separation of the elements, with the faster elements reaching the detection location 626 first and the slowest reaching the detection location 628 last. The different times at which the signals are generated and form the electropherogram indicate the size of the element behind that signal.

It is possible to assist in the interpretation of the unknown element sizes by using a size standard within the capillary. This is provided with a different dye colour or otherwise rendered distinct. The method set out in U.S. patent application No. 61/096,424, the contents of which are hereby incorporated by reference, offers approaches for determining the sizes of the unknowns from the size standard.

The setup and operation of the light source, optics and detector is described in detail below.

Other embodiments of the cartridge have also been developed.

As shown in FIG. 27, the cartridge 27-01 has been modified by providing the electrochemical pumps 27-03, 27-05, 27-07, 27-09 with connections between the wires leading to the electrodes in the pumps and the power source not shown of the Pogo™ pin type. The pins 27-11 are spring loaded in the recesses of the cartridge 27-01 and in use contact similar spring loaded pins (not shown) on the other side of the cartridge to instrument interface. A reliable electrical contact is thus provided and the cartridge is more robust against damage during storage, installation and use than designs in which the wires for the electrochemical pumps protruded from the side of the cartridge.

The form shown in FIG. 27 also features guide holes 27-13 which are used in the alignment of the cartridge and instrument, as described in more detail below.

A preferred embodiment of the cartridge is shown in FIG. 28a. This is an illustration of that part of the sample receiving step 200 provided on the cartridge 28-09, the whole sample preparation step 202, the whole sample amplification step 204, the whole sample denaturation step and the feed to the capillary electrophoresis step 206.

FIG. 28b provides details of the volumes of the various chambers used, the depths (into the page in effect) for the various components and the overall dimensions of this part of the cartridge 28-09.

The cartridge 28-09 is provided with a sample introduction chamber 28-302 connected to a channel 28-304 leading to the outside of the cartridge 28-09. This forms those parts of the sample receiving step 200 provided on the cartridge 28-09.

The sample preparation step 204 follows. To provide this, the sample introduction chamber 28-302 is connected to a pumping fluid channel 28-306 and hence to a first electrochemical pump 28-308. The sample introduction chamber 28-302 has an outlet channel 28-310 which passes valve 28-312 and provides an inlet to bead storage chamber 28-318. Valve 28-312 is initially open.

The bead storage chamber 28-318 has an outlet channel 28-316 leading to binding buffer storage chamber 28-314. This sequence of chambers is reversed compared with the FIG. 3a embodiment. The binding buffer storage chamber 28-314 has an outlet channel 28-320 which leads to mixing/purification chamber 28-322.

Mixing/purification chamber 28-322 is connected via channel 28-324 through valve 28-326 and via channel 28-500 to first further mixing chamber 28-332. The outlet channel 28-324 from mixing/purification chamber 28-322 is blocked by closed valve 28-326, but a vent channel 28-328 is open because valve 28-330 is open initially.

The outlet channel 28-324 leads past valve 28-326 to a first further mixing chamber 28-332 and then through channel 28-334 to second further mixing chamber 28-336. The outlet 28-338 from the second further mixing chamber 28-336 leads past valve 28-340 to incubation chamber 28-342, where bubble mixing assists the DNA to bead binding process. The incubation chamber 28-342 may be actively heated or may simply provide the necessary dwell time and/or other binding conditions needed.

The incubation chamber 28-342 has a vent channel 28-344 provided with valve 28-346 and an outlet channel 28-348 which is initially closed by valve 28-350. The incubation chamber 28-342 is also provided with a pumping fluid inlet channel 28-352 which passes valve 28-354 and is connected to second electrochemical pump 28-356.

The outlet channel 28-348 from the incubation chamber 28-342 leads to capture chamber 28-358 where the beads and hence bound DNA are collected. The capture chamber 28-358 is provided with a first vent channel 28-360 which passes first valve 28-362 and second valve 28-364. The capture chamber 28-358 is also provided with a second vent channel 28-366 which passes first valve 28-368 and second valve 28-370.

Also connected to capture chamber 28-358 is wash buffer channel 28-372. The wash buffer channel is connected to first valve 28-374 and second valve 28-376 and leads from second electrochemical pump 28-356 through wash buffer chamber 28-378 to the capture chamber 28-358.

Also connected to capture chamber 28-358 is an elution liquid channel 28-380. The elution liquid channel 28-380 is connected to first valve 28-382, elution liquid storage chamber 28-384, second valve 28-386 and back to third electrochemical pump 28-388.

The capture chamber 28-358 has a wash outlet channel 28-390 which splits into a first wash outlet channel section 28-392 which passes valve 28-394, and into a second wash outlet channel section 28-396 which passes valve 28-398. After passing their respective valves 28-394, 28-398, the first wash outlet channel section 28-392 and second wash outlet channel section 28-396 rejoin one another to form further wash channel 28-400. The further wash channel 28-400 leads past valve 28-402 into waste chamber 28-404. The waste chamber 28-404 is vented along vent channel 28-406 past valve 28-408. These elements provide the sample preparation step 202.

To provide the sample amplification step 204, capture chamber 28-358 is also provided with elution outlet channel 28-410 which leads past valve 28-412 and past valve 28-414 and past valve 28-502 and into PCR chamber 28-416. The outlet channel 28-418 from the PCR chamber 28-416 leads past valve 28-420 and past valve 28-504 and past valve 28-506 into archive chamber 28-422. The archive chamber 28-422 is vented through vent channel 28-424. The role of the archive chamber 28-422 is as described further above.

Provided within the PCR chamber 28-416 is a bead loaded with the reagents, a multimix, needed for the PCR process. The reagents/multimix include primers dNTPs and PCR reaction mix, including Tris buffer, MgCl₂, NaCl and BSA. These reagents are released into the sample once it contacts the bead in the PCR chamber 28-416 and the temperature is raised above ambient temperature.

The above circuit overall, is sufficient to receive, retain, wash, elute and perform PCR on the sample, as well as storing the waste from the process and an archive of the PCR product.

The PCR part of the circuit has been moved to the upper section of the cartridge compared with the previous embodiments so as to present it physically closer to the CE chip.

Subsequently, the further arrangement shown in FIG. 28a can be used to prepare, denaturation step, and transfer the now amplified DNA from the PCR chamber 28-416 into the electrophoresis step 206.

Leading from the PCR chamber 28-416 is outlet channel 28-418. This splits after valves 28-420 and 28-504 into a denaturing feed channel 28-550 and the channel leading to the archive chamber 28-422. The denaturing feed channel 28-550 is connected to a denaturation chamber 28-552. The amplified material is pumped from PCR chamber 28-416 by the action of fourth electrochemical pump 28-554 which is connected to channel 28-556, hence to denaturing reagent storage chamber 28-558 and through valve 28-560 and channel 28-562 to the PCR chamber 28-416. Formamide is provided in the denaturing reagent storage chamber 28-558 in combination with the size standards to be used in the capillary electrophoresis step.

These components are isolated from the PCR chamber 28-416 during the sample amplification step 204 by closed valve 28-502 and closed valve 28-420. Both valve 28-502 and 28-420 are opened and valves 28-414 and 28-506 are closed to convey the amplified material away from the PCR chamber 28-416 to the denaturation chamber 28-552. This is vented through valve 28-564, with exit channel 28-566 closed by valve 28-568.

The amplified material is held in the denaturation chamber 28-552 for the necessary time and at the necessary temperature to complete the denaturing process. Once this has been achieved, the valve 28-568 is opened and further pumping by the fourth electrochemical pump 28-554 pumps the denatured material to the electrophoresis step inlet 28-570.

At the inlet 28-570, the denatured material passes out of the plane of the cartridge 9 and through a tube to the electrophoresis cartridge section behind. The overall result of this structure is the pumping of the amplified DNA to a start point for the electrophoresis step 206.

Details of the connection of the inlet 28-570 to the CE chip are provided below.

Throughout the operations described above and in the sections that follow, various checks are made on operating conditions, component performance and successful operation so as to ensure the processing is correctly provided from start to finish. Errors or problems are indicated to the operator.

### Cartridge Sequence of Operation

The sequence of operation, purely by way of example, applied to the cartridge shown in and described in relation to FIG. 3a and b is as follows, with sample timings also given.

| Time | | Purpose and |
|---|---|---|
| since start (sec) | Change | notes |
| 0.0 | Incubation chamber 358 - adjust | |
| | temperature to 25° C. | |
| 0.9 | Valve 312 - opening valve - heat on | |
| 31.5 | First electrochemical pump 308 - on | |
| 73.3 | Valve 330 - closing valve - heat off | |
| 121.1 | Valve 312 - opening valve - heat off | |
| 138.7 | First electrochemical pump 308 - off | |
| 187.8 | Valve 326 - opening valve - heat on | |
| 212.3 | Valve 312 - opening valve - heat on | |
| 233.9 | Valve 330 - closing valve - heat off | |
| 236.0 | First electrochemical pump 308 - on | |
| 324.3 | Valve 312 - opening valve - heat off | |
| 368.6 | Valve 326 - opening valve - heat off | |
| 370.4 | Valve 346 - closing valve - heat on | |
| 401.0 | First electrochemical pump 308 - off | |
| 461.4 | Valve 346 - closing valve - heat off | |
| 653.4 | Valve 350 - opening valve - heat on | |
| 655.1 | Magnet - field applied to chamber | |
| 656.4 | Valve 326 - opening valve - heat on | |
| 684.5 | First electrochemical pump 308 - on | |
| 783.4 | Valve 326 - opening valve - heat off | |
| 804.1 | Valve 394 - closing valve - heat on | |
| 815.4 | Valve 340 - closing valve - heat on | |
| 829.6 | Valve 350 - opening valve - heat off | |
| 840.8 | Magnet - field removed from | |
| | chamber | |
| 867.5 | First electrochemical pump 308 - off | |
| 894.2 | Valve 394 - closing valve - heat off | |
| 944.5 | Valve 368 - opening valve - heat on | |
| 975.5 | Valve 340 - closing valve - heat off | |
| 977.2 | Second electrochemical pump 356 - | |
| | on | |
| 1025.8 | Valve 354 - closing valve - heat on | |
| 1036.2 | Valve 368 - opening valve - heat off | |
| 1050.8 | Second electrochemical pump 356 - | |
| | off | |
| 1079.7 | Valve 324 - opening valve - heat on | |
| 1080.6 | Valve 368 - opening valve - heat on | |
| 1116.3 | Valve 354 - closing valve - heat off | |
| 1118.0 | Second electrochemical pump 356 - | |
| | on | |
| 1181.3 | Valve 370 - closing valve - heat on | |
| 1196.4 | Valve 368 - opening valve - heat off | |
| 1228.3 | Valve 324 - opening valve - heat off | |
| 1233.9 | Second electrochemical pump 356 - | |
| | off | |
| 1244.2 | Valve 398 - opening valve - heat on | |
| 1249.4 | Valve 324 - opening valve - heat on | |
| 1271.8 | Valve 370 - closing valve - heat off | |
| 1273.1 | Magnet - field applied to chamber | |
| 1284.7 | Second electrochemical pump 356 - | |
| | on | |
| 1328.6 | Valve 324 - opening valve - heat off | |
| 1333.8 | Valve 402 - closing valve - heat on | |
| 1334.7 | Valve 408 - closing valve - heat on | |
| 1379.9 | Valve 398 - opening valve - heat off | |
| 1383.8 | Magnet - field removed from | |
| | chamber | |
| 1393.9 | Second electrochemical pump 356 - | |
| | off | |
| 1419.5 | Valve 362 - opening valve - heat on | |
| 1435.4 | Valve 402 - closing valve - heat off | |
| 1465.1 | Valve 408 - closing valve - heat off | |
| 1466.0 | Second electrochemical pump 356 - | |
| | on | |
| 1474.6 | Valve 374 - closing valve - heat on | |
| 1493.6 | Valve 362 - opening valve - heat off | |
| 1501.8 | Valve 382 - opening valve - heat on | |
| 1504.8 | Valve 362 - opening valve - heat on | |
| 1508.7 | Second electrochemical pump 356 - | |
| | off | |
| 1531.9 | Third electrochemical pump 388 - on | |
| 1578.8 | Incubation chamber 358 - adjust | |
| | temperature to 60° C. | |
| 1585.0 | Valve 374 - closing valve - heat off | |
| 1586.6 | Valve 362 - opening valve - heat off | |
| 1588.5 | Valve 364 - closing valve - heat on | |
| 1633.3 | Valve 382 - opening valve - heat off | |
| 1640.4 | Third electrochemical pump 388 - off | |
| 1679.0 | Valve 364 - closing valve - heat off | |
| 1881.0 | Valve 412 - opening valve - heat on | |
| 1882.9 | Valve 382 - opening valve - heat on | |
| 1906.2 | Magnet - field applied to chamber | |
| 1914.9 | Third electrochemical pump 388 - on | |
| 1952.3 | Incubation chamber 358 - adjust t to | |
| | 25° C. | |
| 2010.0 | Third electrochemical pump 388 - off | |
| | Magnet - field removed from | |
| | chamber | |
| | Valve 382 - opening valve - heat off | |
| | Valve 412 - opening valve - heat off | |
| 2017.3 | Valve 420 - closing valve - heat on | Isolate PCR |
| | Valve 414 - closing valve - heat on | chamber |
| 2173.3 | Valve 420 - closing valve - heat off | |
| | Valve 414 - closing valve - heat off | |
| 2185.0 | Incubation chamber temperature | |
| | control - off | |

### Additional Features for Use in the Invention

### Cartridge Alternatives

There are a variety of alternatives for the various components within the cartridge and/or their operation. Some of these are now described, by way of example only.

### 1) Bead Handling

As described above, the cartridge makes use of a bead storage chamber 318 from which the beads are washed in operation. This washing action provides contact between the sample, reagents and the beads. Mixing results in the beads taking up the DNA in the sample and retaining it. Subsequent retention of the beads allows the DNA to be separated from the rest of the sample and allows washing stages to improve further this separation.

It is important to ensure that the beads are displaced from their storage location, such that the beads are available, in contact with the relevant liquids, to perform their task. Modifications to the manner in which the beads are stored and/or dispensed can assist in this. The beads may be stored away from the cartridge. They may be introduced to the cartridge to prepare it for use.

Firstly, it is possible to provide a dispersant together with the beads so as to keep them dispersed and hence more easily collected and carried by the fluid flow. This can help prevent blockages and/or agglomerations of beads. Different dispersants and/or variations in the amount provided can be used to tailor this.

Secondly, it is possible to provide the beads in a series of bead storage chambers, rather than in a single chamber. FIG. 6a illustrates one such arrangement, where the beads are split into three groups, each in its own chamber 700. In this way, the contact between the fluid and the beads is staggered and a compacted mass of beads is avoided on the lead edge of the fluid. A variation on this is provided in FIG. 6b, where a first bead storage chamber 700a is separated from the second bead storage chamber 700b by a mixing chamber 702.

Thirdly, the contact can be provided with a thin chamber 704 whereby the transition of the fluid from the thing channel 706 into the chamber causes non-laminar flow and hence improved mixing. The provision of the beads spread along the length of the chamber 704 also means that they do not contact the fluid all at the same time.

Fourthly, the flow direction and/or chamber design can be modified to encourage displacement of the beads from their storage position into a mixed form with the fluid. Thus in the FIG. 6d form, the fluid enters the chamber 700 in one bottom corner 708 and displaces, arrows, the beads resting in that part. A swirling flow within the chamber 700 gives mixing, before the fluid and bead mixture exits the chamber 700 through the other bottom corner 710.

Fifthly, the beads can be stored in a side arm 712 or other form of passage. As the flow of fluid passes through thin chamber 714 and past the junction 716 with the arm 712, a force is applied behind the mass of stored beads in the side arm 712. This forces the mass of stored beads towards and into the junction 716 where they gradually contact and are swept away by the fluid flow. Gradual dispersal of the beads into the fluid is provided. The motive force behind the beads can be provided by a similar structure to that used to move material in the context of the closing valves described herein.

### 2) PCR Chamber Filling

In the above system, the amount of the processed sample which is made available to the PCR stage is controlled by the relative height of the outlet from the PCR chamber to the archive chamber leading to overflow of excess sample into the archive chamber. This results in a PCR chamber which is not completely full of sample during PCR. As PCR involves heating of the sample, evaporation and/or condensation of part of the sample may occur at a location outside of the PCR chamber. This can reduce the reagents present in the PCR chamber and hence reduce the efficiency of the PCR stage.

In an alternative form, the PCR chamber is entirely filled with the sample before PCR is started. This is achieved using the arrangement of FIG. 7 where the majority of the components have the same structure and function as shown in the FIG. 3 and FIG. 4 description. The differences are in the section around the PCR chamber 416.

In this alternate form, the PCR chamber 1416 is fed material along channel 1413. Initially, the path of least resistance to this fluid flow is through the PCR chamber 1416, along channel 1500, past opened valve 1502 and onto vent 1504. The vent 1504 is hydrophobic and so allows the passage of the air displaced from the PCR chamber 1416 and channel 1500 by the material's advance. Once the fluid reaches the vent 1504, however, the path of least resistance changes and further flow occurs along channel 1418 past valve 1428 and into archive chamber 1422, which is provided with vent 1424. By this time, the PCR chamber 1416 is completely full of liquid and hence the volume of the liquid subjected to PCR is guaranteed.

As before, the valves around the PCR chamber 1416 are closed during the amplification itself, so as to isolate the PCR chamber 1416.

In a third alternative, the configuration shown in FIG. 22, the PCR chamber 22-01 is along channel 22-03. Initially, the path of least resistance to this fluid flow is through the inlet 22-05 to the PCR chamber 22-01. Once the PCR chamber 22-01 has filled, the liquid overflows through exit 22-07 into channel 22-09 which is a continuation of channel 22-03. Further fluid flow simply by-passes the PCR chamber 22-01 and flows through channel 22-03 and then channel 22-09. To control the flow correctly, the dimension A of the inlet 22-05 is greater than the dimension B of the outlet 22-07. The dimension is preferably greater in terms of the cross-sectional area, perpendicular to the direction of flow. The complete filling of the PCR chamber 22-01 ensure the volume of the liquid subjected to PCR is guaranteed.

Various shapes are possible for the PCR chamber. FIG. 26 provides an example in which the PCR chamber 26-01 is formed as smooth as possible. This assists with full fluid contact with the surfaces and hence complete and accurate filling of the PCR chamber 26-01. The sample flows along channel 26-03 and enters the PCR chamber 26-01 via inlet 26-05 provided towards the bottom of the PCR chamber 26-01. The sample fills the PCR chamber 26-01 before overflowing through outlet 26-07 provided towards the top of the PCR chamber 26-01 and into channel 26-09.

In the embodiment of FIG. 46, a variation on the above principle is provided. The flow to the PCR chamber 46-100 passes along channel 46-102 and past valve 46-104. The channel 46-102 turns as it approaches the chamber 46-100 and provides inlet channel 46-106. The natural flow is along this route. As the flow progresses, the PCR chamber 46-100 fills, with the gas exiting through outlet channel 46-108. The outlet channel 46-108 has a similar configuration to inlet channel 46-106, but the cross-sectional area of the outlet channel 46-108 is much smaller than that of the inlet channel 46-106. As a result, when the liquid reaches the outlet channel 46-108, the flow resistance increases greatly and flow is redirected along the by-pass channel 46-110 in preference. Both the outlet channel 46-108 and the by-pass channel 46-110 lead past valve 46-112 to exit channel 46-114. The Peltier effect device heats the area within the dotted lines and so ensures that as much of the space between the two valves, 46-104 and 46-112 is heated so as to minimise any condensation within that space.

### 3) Sample Concentration Before Capillary Electrophoresis

In some instances, it may be helpful to increase the concentration of the sample prior to its use in the electrophoresis step and/or to reduce the size of the sample as it is injected.

Once suitable approach for doing so is set out in European patent publication no 1514100, the contents of which are incorporated herein by reference. This technique uses careful balancing of the electrophoretic velocity of the DNA and the opposing electroosmotic velocity to concentrate the DNA at the liquid to gel interface. A change in conditions can then be used to drawn the concentrated DNA into the electrophoresis step as a concentrated and small sample.

Another option is hydrodynamic stacking. This is based upon the variation in the flow velocity between sample and the location from which the size based separation starts, for instance through the use of adjustments to conductivity, buffer components, pH and the like. An example of such an approach is field amplified sample stacking, FASS. This provides higher electric fields in the lower conductivity zones than in the higher conductivity zones. The sudden potential drop at the interface between the two zones causes sample stacking there.

Mechanical pre-concentration is also a possibility. Packed beds, nanochannels, immobilised polymers and membranes all offer the possibility of trapping and concentrating the sample. Electro-elution, where by the release of the sample is caused by the application of an electric potential to a membrane, is one possibility.

A combined technique approach to pre-concentration may be particularly beneficial. Such an approach is shown in FIG. 24, in the case of CE channel being in the same plane as the rest of the cartridge, and FIG. 25, in the case of the CE channel not being in the same plane as the rest of the cartridge.

As illustrated, the combined flow 24-01, 25-01 of DNA containing sample and formamide pass valve 24-03, 25-03 and then reach a junction 24-05, 25-05. The Y-shaped junction brings the combined flow 24-01, 25-01 into proximity with the running buffer flow 24-07, 25-07 in channel 24-08, 25-08. These flows cross the CE channel 24-09, 25-09 and any excess passes to chamber 24-11, 25-11. The left-hand detail shows the construction present at the intersection of the CE channel 24-09, 25-09 and the channel 24-08, 25-08.

In the FIG. 24 form, the stacking interface 24-11 is provided between the combined flow 24-01 and buffer flow 24-07. The electric potential is provided by electrode 24-13. The second stacking function is provided by the membrane 24-15 provided between the buffer flow 24-07 and the CE channel 24-09.

In the FIG. 25 form, the stacking interface is similarly provided.

### 4) Alternative Electrophoresis Channel Configuration

In the embodiment described above, the injector is of the double T type. As an alternative, it is possible to use a cross-channel injector, as shown in FIG. 8.

In this case, the reservoir 604, channel 610 and other parts leading to the fourth electrode location 620 are the same. The arm 624 provided with the fourth electrode location 620 and the arm 622 provided with the third electrode location 618 are aligned on a common axis and at 90° to the main capillary 616.

The sample is drawn towards the electrode at the third electrode position 618 by the application of a voltage. To prevent dispersion of the sample into the main capillary, towards the first 612 and/or second 614 electrode locations, a voltage is applied to the electrode at the first electrode location 612 and to the electrode at the second electrode location 614. This has the effect of pinching the part of the sample at the intersection of the main capillary 616 and the arms 622, 624, and maintaining the minimal size of the plug which is then used in the capillary electrophoresis.

A further electrophoresis channel configuration is shown in FIG. 43. In this case, the sample flows along channel 43-100 from inlet 43-102 to outlet 43-104. A potential difference is applied between locations A and B. This draws the DNA in the sample towards the membrane 43-106. The membrane is sized, 10-14 kDa cutoff, to retain the DNA. The separation matrix is then flowed into the channel 43-100; UV activation may be provided, as discussed elsewhere. The same buffers at location A, B and in the matrix are then provided for the electrophoretic separation to be provided through the application of a potential difference between A and B.

The polarity may be provided in the reverse direction before the CE run, for instance to ensure the buffer extends from A to B. DNA is not lost as the flow will maintain it on the membrane 43-106.

Between loading to the membrane 43-106 and the CE separation, it is possible to introduce a variety of reagents/buffers into locations A and/or B and/or the channel 43-100 to assist in purifying the DNA and/or to optimise CE conditions, for instance through removal of excess salts and/or unincorporated PCR primers. Both locations A and B have their own inlets and outlets for this purpose.

A still further configuration is shown in FIG. 44. In this case, again the sample flows through channel 44-100 from inlet 44-102 to outlet 44-104. A potential difference between A and B is used to attract and retain the DNA on a membrane 44-106. By swapping to an electrolyte flow through channel 44-100 and changing the potential difference it is possible to load the DNA to the matrix in main channel 44-108. The CE can then be performed.

Again one or more cleaning or condition controlling steps may be provided before CE is conducted.

A yet further configuration is shown in FIG. 45. In this case, the arm 45-100 leading the sample into the main channel 45-102 where CE is performed extends downwards, at least partially aligned with gravity. The arm 45-104 leading away from the main channel 45-102 extends upward, at least partially aligned with gravity. In this way gravitation effects promote retention within the main channel 45-102, rather than encouraging flow away from it and into another arm.

### 5) Cartridge Variant for Real Time PCR Performance

In the cartridge 9 described above, the cartridge 9 is being used to consider a reference sample. In this alternative embodiment, the changes to the cartridge 5009 beneficial to the consideration of a casework sample are considered.

A major difference between a casework sample and a reference sample is that whilst the amount of DNA recovered in a reference sample has a degree of consistency, and is of a high level, this is not the case for a casework sample. The manner in which the sample is left, the passage of time, the collection process and other factors can all result in the amount of DNA in a casework sample being unpredictable, and often lower, than desired.

To counteract this, the casework sample processing seeks to ensure that the amount of DNA arising from the amplification process is within certain bounds.

To do this, the casework sample provides for parallel processing of the sample, particularly in terms of the sample amplification step 204.

The sample receiving step 200 and sample preparation step 202 are basically the same as previously described. The difference comes in the sample amplification step 206.

The channel 5410 containing the eluted DNA from the beads held in the incubation chamber 5358 leads to a junction 5700 where the flow is split into two separate streams 5702, 5704.

The first stream 5702 passes into a PCR chamber 416 of the type previously described (and is not illustrated further). The subsequent handling of this by the cartridge 9 is as described above, save for the possible changes in the sample amplification conditions/duration described shortly.

The second stream 5704 passes into a second separate PCR chamber 5706. This second PCR chamber 5706 contains a bead provided with a coating containing the necessary regents for PCR and for a quantification analysis.

During processing, PCR is advanced in the PCR chamber 416 and in the second PCR chamber 5706, in parallel. After a given number of PCR cycles for the second PCR chamber 5706, the contents of the second PCR chamber 5706 are considered to establish the quantity of DNA which has been generated by the PCR cycles up to that point. This can be equated to the amount of DNA present within the original sample and hence the amount of DNA the PCR chamber 416 is working on. As a result of the quantification, the PCR conditions and/or cycle number for the PCR chamber 416 can be varied to optimise the quality of amplification product.

Further details on the operation of such a system and the use of this feed back are to be found in 61/026,869, the contents of which are incorporated herein by reference, particularly as they relate to the parallel conduct of PCR and the use of the results from one PCR to control and/or modify the conduct of the other PCR.

Suitable reagents include the Plexor HY kit available from Promega Inc, 2800 Woods Hollow Road, Madson, Wis. 53711, USA and Quantifiler® Duo DNA quantification kit available from Applied Biosystems, Foster City, Calif., 944404, USA.

To establish the quantity of DNA present, it is necessary to interrogate the sample using an excitation light source and then quantify the amount of light arising. To do this, light from a light source is conveyed to the second PCR chamber 5706 and focussed thereon using a lens system. The excitation light interacts with the dye(s) associated with the sample. The fluorescent light generated is detected and is proportional to the quantity of DNA present.

The light source used could be the same light source as is used for the electrophoresis step 206, and described in detail below. The light would be conveyed to the second PCR chamber 5706 by an optical fibre. Because the Peltier heater/coolers are positioned in front of and behind the second PCR chamber 5706, the light for the detection is introduced from the side of the cartridge 9. The light source may be a laser, for instance of the type and/or with the set up discussed further below in the electrophoresis step 206. As an alternative, however, it is possible to use a light emitting diode based light source, as described below.

Depending upon the quantity, the number of cycles used in the PCR chamber 416 may be increased, decreased or kept at the normal level, so as to provide a quantity of DNA within the desired range after PCR has been completed in PCR chamber 416.

In the context of real time quantification and/or the handling of samples from crime scenes (rather than those taken under controlled conditions from individuals), differences in the implementation of the invention may be provided. These may include:

1) The parallel processing of the sample so as to allow the results from a first processing of the sample to inform on the optimum conditions etc to be used in the main processing of the sample. Further details of such an approach are to be found in WO2009/098485, the contents of which are incorporated herein by reference with respect to the parallel processing and consideration of samples and the feedback of information from one processing to the other.

2) The efficiency of the extraction should be as high as possible, for instance through optimised sample recovery, lysis and amplification. The use of various processes and/or reagents to separate the DNA of interest from problematic components, such as PCR inhibitors, is beneficial in this respect.

3) The cartridge used will feature many of the steps and components exemplified above, but with the incorporation of the parallel PCR circuit and the ability to analyse the results there from, for instance using a laser or LED to apply light to the liquid, with the return light being detected to inform on the PCR process. Photo diodes and/or cameras can be used in the light detection. A control material may be provided within the sample to provide a reference value with respect to the light detected.

4) The instrument would benefit from being able to run positive and/or negative controls. These could be run in the same cartridge as the sample. The controls may be handled by the operator in the same manner as the sample of interest so as to inform on contamination risks. The controls may just be run periodically so as to check on the instrument, for instance in the form of a calibration check.

### Cartridge Components

Within the cartridge are a significant number of components, with each being optimised with respect to its role and its role in combination with the other components.

### 1) Valves

To minimise manufacturing costs and give consistent operation, all of the valves in the cartridge are one of two types. The two types are a closing valve 2000; FIG. 10a; and an opening valve 2002; FIG. 10b.

The closing valve 2000 is shown schematically in FIG. 10a. The closing valve 2000 is positioned above, relative to the direction of gravity, the channel 2004 to be closed. The closing valve 2000 is formed by a conduit 2006 which is in fluid communication with the channel 2004 and is in fluid communication with the bottom of a valve reservoir 2008. The valve reservoir 2008 is filled with paraffin wax and is 3 mm in diameter and is provided with the conduit 2006. On the top of the valve reservoir 2008, a gas passage 2010 provides fluid communication with a valve gas reservoir 2012. The valve gas reservoir 2012 is full of air.

The dotted line in FIG. 10a shows that part of the location of the closing valve 2000 which is in contact with a heater element, not shown, provided on the adjoining printed circuit board of the instrument.

When the closing valve 2000 is to be activated, the heater element is caused to heat up. This both melts the paraffin wax in the valve reservoir 2008 and causes the air in the valve gas reservoir 2012 to expand. The expansion of the air provides the driving force to displace the melted paraffin wax from the valve reservoir 2008 into the conduit 2006 and then into the channel 2004.

The volume of paraffin wax displaced is controlled by the temperature to which the valve gas reservoir 2012 is heated (variation in pressure) and the duration of the heating applied (as the paraffin wax soon solidified once the heating is switched off).

Continued displacement of the paraffin wax into the channel 2004 causes the paraffin wax to expand in each direction along the channel 2004.

In some cases, the fluid in the channel will not compress or move in one direction (or is limited in the extent possible) and so the flow of the paraffin wax within the channel 2004 occurs preferentially in the other direction. Normally, the paraffin wax is displaced into the channel 2004 until a 2 mm to 10 mm length of the channel 2004 is filled. With the heat removed, the paraffin wax sets in this new position and the channel 2004 is reliably sealed.

The section where the channel 2004 is to be shut, is deliberately chosen to be horizontal, relative to the direction of gravity, as this assists the retention of the paraffin wax at the location to be sealed.

To assist further in the formation of the seal, it is beneficial to arrange the closing valve so that it is between one or two upward, relative to the direction of gravity, bends. As shown in FIG. 10a the bend 2014 provides assistance in the accurate formation of the seal within the channel 2004.

The opening valve 2002 is shown schematically in FIG. 10b. The opening valve 2002 is positioned as a part of the channel 2004 the fluid flows through. The opening valve 2004 is formed by a valve chamber 2020 which has an inlet 2022 from the channel 2004 in a first side wall 2024 and an outlet 2026 leading to the continuation of the channel 2004 in the opposing side wall 2028.

The paraffin wax is positioned in the initial section 2030 of the valve chamber 2020. Downstream of this initial section 2030, is a trap section 2032. The dotted line in FIG. 10b shows that part of the opening valve 2002 which is in contact with a heater element, not shown, provided on the adjoining printed circuit board of the instrument.

When the opening valve 2002 is to be activated, the heater element is caused to heat up. This melts the paraffin wax in the initial section 2030. By the time the paraffin is melted, or shortly thereafter, an electrochemical pump upstream of the opening valve 2002 has been activated for sufficient time to cause a pressure build up, upstream of the opening valve 2002. This pressure causes the driving force to displace the melted paraffin wax from the initial section 2030 and downstream into the trap section 2032. Once in the trap section 2032, the passage 2034 above the paraffin wax is clear allowing fluid communication through the opening valve.

With the heat removed, the paraffin wax sets in this new position and the channel 2004 and passageway 2034 is reliably opened.

The section where the channel 2004 is to be opened is deliberately chosen to be horizontal, relative to the direction of gravity, as this assists the retention of the paraffin wax in the trap section 2032.

In some applications, particularly those close to the high temperatures used in the PCR chamber, the valves benefit from using a high melting point wax. This melts at greater than 95° C. and so does not melt under PCR conditions. In some cases, the valve performance can be improved further by using a high melting point and lower melting point mixture; with the lower melting point wax tending to fill any cracks which form in the higher melting point wax.

A further valve embodiment is shown in FIG. 47. The channel 47-100 is connected to the valve by a side channel 47-102 as usual. The side channel 47-102 leads to a first chamber 47-104. This is connected via a short channel 47-106 to a larger second chamber 47-108.

### 2) Chambers

Within the cartridge, a variety of chambers are provided for a variety of purposes. To achieve those purposes efficiently and effectively, the chamber designs are optimised in various ways.

With respect to the incubation chamber 358, this is provided with a broad base which is generally horizontal. In operation, the offset magnet (not shown) is used to restrain the magnetic beads in position during washing and during elution. The broad base provides a suitable location to which the beads can be drawn and secured, whilst exposing them to the wash flow or to the elution flow.

The sloping walls within the incubation chamber 358 and the bubble mixing chamber 342 are provided to promote the flow of eluent, introduced into the chambers at the top, to the outlet at the bottom of the chamber.

The angular corners are used to generate improved pressure gradients from the inlet for a part of the process to the outlet in that respective part of the process.

The first further mixing chamber 332 and second further mixing chamber 336 are provided to encourage non-laminar flow within the flow route. As the fluid transitions from the channel, with its cross-section, to the chambers, with their increased cross-section, non-laminar flow arises. This gives good mixing for the different density fluids and particles which are all to be mixed. Such mixing forms are significantly better in this respect than bubble mixing alone or piezoelectric based mixing.

The PCR chamber 416 has two principle embodiments; as described above. In each, the PCR regents are provided within the degradable shell of a bead located within the PCR chamber 416. To ensure proper flow of the liquids around and past the bead, the bead is provided with a bead seat. This provides a defined rest position for the bead, but as the bead is only contacted at discrete locations when in the seat, fluid is still able to flow past the bead. The seat ensures that the bead does not block at inlet to and/or outlet from the PCR chamber 416. The seat ensures that there are no large areas of the bead surface, and hence of the reagents, which are isolated for fluid contact.

In the second of the PCR chamber 416 embodiments, described in the alternatives for the cartridge section, the PCR chamber 416 is completely filled with fluid. This gives a reproducible volume of fluid in the PCR process. The same position arises with the third embodiment, FIG. 22.

In the first of the PCR chamber 416 embodiments, the maximum level of fluid within the PCR chamber 416 is controlled by the relative height of the outlet within the chamber. The outlet in effect acts as an overflow for the fluid, once the PCR chamber 416 has filled to this level. A head space remains above the fluid, within the PCR chamber 416.

### 3) Vents

To allow fluid flow, air or sample, around the cartridge 9, various vents need to be provided for various chambers.

To prevent any risk or suggestion that material can enter the cartridge 9 through such vents, each of the vents is provided with a filter element to exclude particulate material. In addition, when a vent is part of the active processing on the cartridge 9, the vent is under positive pressure and so air is flowing out through the vent. This too assists in preventing any risk of particulate material entering the cartridge 9.

In some situations, it is desirable to be able to allow air to pass through the vent freely, but for the vent to resist the passage of any subsequent liquid. An example is to be found in the alternative PCR chamber 416 filling embodiment. To provide this, those vents are hydrophobic. The vent may be hydrophobic because of the base material forming the vent and/or because of a treatment applied to the material of the vent. Such a treatment can be provided, for instance, by using polypropylene material and/or by providing a polysulphone coating.

### 4) Archive

As described above, the fluid not needed in the PCR chamber 416, is pumped onward to an archive chamber 422.

The purpose of the archive chamber 422 is to provide a storable record of the sample supplied to the sample amplification stage 204, and the PCR chamber 416 in particular.

If needed, the sample in the archive chamber 422 can be accessed at a later date to enable a further amplification and analysis to be performed. Further processing in this way is useful where it is necessary to repeat the analysis, for instance by way of verification. Alternatively, further processing enables a different amplification and analysis protocol to be applied, for instance, a protocol suitable for low levels of DNA within the sample.

In the form shown in FIG. 3, the archive chamber 422 is an integral part of the overall cartridge 9.

In an alternative, form shown in FIG. 11, the archive chamber 2422 is still fed the surplus sample through a channel 2418 leading away from the PCR chamber, not shown.

The archive chamber 2422 is positioned on a stub 2750 which extends from the side of the cartridge 9. The stub 2750 is connected to the cartridge 9 during normal use, but a line of weakness 2752 is provided. This allows the stub to be snapped off the cartridge 9 after the completion of the processing. This means the archive function can be provided by only storing the stub 2750, rather than have to store the far larger overall cartridge 9. Given the number of samples which may be considered, and the time for which they have to be stored, saving of storage space is a significant issue.

To seal the archive chamber 2422, once it has been loaded, a closing valve 2754 is provided on the cartridge 9 side of the line of weakness 2752 and a further closing valve 2756 is provided on the stub 2750 side of the line of weakness 2752. These valves are activated to place paraffin wax in the channel 2418 on either side of the line of weakness 2752. To provide for long term storage, a further closing valve 2758 is provided on the channel leading from the archive chamber 2422 to the vent 2424.

Just as the cartridge 9 is provided with an identifier, which is used to link it in the records to the sample loaded upon it, then the stub 2750 is also provided with a common identifier so as to maintain the link after the stub 2750 is broken off the cartridge 9.

### 5) Reagents

Various options exist for the provision of the reagents needed in the various steps of the processing. As far as possible, so as to keep the processing as simple as possible for the user, the cartridge 9 is provided with pre-loaded reagents. Examples of such pre-loaded reagents would include the bead provided in the PCR chamber 416; with the bead carrying the PCR regents inside. Other pre-loaded regents include the various wash liquids and elution liquids described in the methodology above.

If necessary, one or more reagents can be provided separate from the cartridge 9, and be loaded onto the cartridge at or close to the time of use. This may be necessary where the reagent is unable to withstand prolonged storage under the conditions to which the cartridge 9 is exposed. These may be conditions of temperature and/or mechanical conditions such as vibration or orientation.

A preferred form of reagent provision is provided where the reagent(s) are provided as part of a solid phase reagent or solid phase reagent storage component, with release of the reagent being triggered by an increased temperature. Gel forms of reagent and/or reagent storage component, preferably triggered to release by the application of higher temperatures are also a useful option.

### 6) Electrochemical Pumps

To simplify the construction and costs of the cartridge, a common approach is used to providing the motive power to the various operations on the cartridge; electrochemical pumps. Each of the electrochemical pumps consists of a pair of electrodes immersed in the electrolyte. The flow of a current results in off gassing. The off gas collects in the top of the electrochemical pump, increases in pressure and leaves the pump via the outlet in the top of the pump. This off gas pushes ahead of itself other fluids encountered in the channels and chambers. The off gas contributes to bubble mixing in some of the stages.

To give a desired extent of pumping, the volume of the electrochemical pump can be varied. The extent of pumping can be delivered in one, two or more goes, as turning off the current stops the pumping action.

The rate of pumping and/or pressure delivered can be varied by varying the molarity of the electrolyte. Sodium chloride is the preferred electrolyte; used at 1M; and used in conjunction with aluminium electrodes.

### 7) Electrophoresis Matrix

The material provided within the capillary of the electrophoresis stage is important to the reliability and resolution of the analysis obtained.

Various possible materials can be used in the capillary. These include the use of polymer matrix, for instance a polyhydroacrylamide, a polydimethylacrylamide or mixtures there of. The polymers may be cross-linked to give the desired properties and/or formed into their state of use within the capillary, after loading. It is also possible to use an inert bed of particulate material to form the matrix in which the size based separation is achieved.

As well as optimising the performance through the properties of the gel, it is also possible to treat the capillary walls to improve properties. For instance it is possible to apply hydrophilic coatings, such as poly(hydroxyethlacrylamide).

A potential methodology for the electrophoresis matrix is to store that material in a chamber which is a part of the CE chip, but not use that chamber for the CE separation.

Instead, when required for use, the stored matrix is moved from the chamber into the capillary so as to fill it to the desired degree. As a result of loading just before use, the matrix is no subject to sedimentation effects; these can have a detrimental effect on the analysis. Pressure loading can be used for this purpose.

Another potential methodology is to fill the main channel and arms of the CE chip with the matrix. Those parts of the CE chip where the matrix is not needed, for instance aside from the main channel, may be masked. In this way, when UV light is applied the parts where the matrix is not needed retain the matrix unaltered. The unaltered matrix can be washed away. Where the matrix is exposed to UV light it is altered and resists washing away.

### 8) CE Chip Design

A preferred configuration for the CE chip is shown in FIG. 42a and the detailed partial view of FIG. 42b.

The end portions 42-100 cooperate with the carrier when the chip is mounted within it. The external profile of the base of the CE chip is designed to match with that defined by the raised surface around the CE chip heater board, described elsewhere in this document.

As described below, a number of electrodes are required in different parts of the channels provided within the CE chip so as to load the sample and then perform the necessary separation to give the analysis. These electrodes within the channels are connected to pins 42-102 which extend above the plane of the CE chip. These pins 42-102 are positioned so that they are within the cut away portion of the second support and so are exposed. This allows suitable connections to be made to these pins 42-102 so as to apply the necessary voltages to them and to the electrodes connected to them.

The CE chip is shown with a single channel in which CE is performed, but channels suitable to perform separations on multiple samples could be provided.

### 9) PCR Chamber Sealing

In the embodiments described elsewhere, the chambers and the valves which are used to seal the channels leading to and from them are separate. In the following embodiment, the chambers and the valves are integrated as a single component.

As shown in FIG. 41a, the PCR chamber 41-100 is provided in the cartridge. However, the walls defining the circumference, at least, of the chamber 41-100 are rotatable within the body of material forming the cartridge. In the left-hand form, the rotatable wall is positioned such that the holes therein are aligned with the inlet channel 41-102 and the loading outlet channel 41-104. As a result, liquid can enter and gas leaves the chamber 41-100 until the chamber is full, centre form. The rotatable wall can then be rotated to align the holes therein with the inlet channel 41-102 and the dispense outlet 41-106, right hand form, to allow the contents to be emptied.

A variant of this approach is shown in FIG. 41b, where inlet channel 41-100 is connected to outlet channel 41-108. Rotation aligns the holes with dispense inlet 41-110 and dispense outlet 41-106.

The variant in FIG. 41c uses the arrangement to seal the chamber during PCR. In the left hand form, the inlet channel 41-102 is connected to and fills the chamber up to the level of the outlet channel 41-108. Partial rotation offsets the holes in the rotating wall from alignment with any of the inlets/outlets, centre form. After PCR, further rotation aligns the holes with the dispense inlet 41-110 and dispense outlet 41-106.

The extent of rotation may be limited by abutment surfaces provide in the cartridge wall which abut surfaces on the rotating walls or vice versa. Partially circular forms for the hole in the cartridge which receives the rotating walls and/or vice versa may also be used to control or limit rotation in one or both directions.

Rotation may be provided by cooperation between an actuator and a slot in the circular wall.

Rotation may cause pads or other pliable material to be compressed or otherwise deformed to give sealing.

One or more of the channels may serve as a light path, rather than or in addition to being a fluid flowpath, so as to allow an investigatory instrument to shine light into the liquid contained within the chamber. Such an embodiment is useful in the context of the cartridge variant for real time PCR discussed above.

### Instrument Configuration and Appearance

The instrument 11 is illustrated in FIG. 12 and is provided within a casing 8000. The mid section 8002 of the instrument 11 is provided with a door 8004 provided with a latch 8006. Behind the door 8004 is the location at which the cartridge 9 is mounted in use. This location is a position in which the plane of the cartridge 9 is parallel to the plane of a printed circuit board 8008. At the location, the cartridge 9 and components on the printed circuit board 8008 contact one another.

Behind the printed circuit board 8008 are the electronics for operating and controlling the components provided on the printed circuit board 8008. These include the power supplies, voltage controllers, temperature controllers and the like.

The upper section 8010 of the instrument 11 provides the display 8012 by means of which the user inputs information into the instrument 11 and receives visual information from the instrument. The software and hardware for operation of the display 8012 are provided on a computer positioned behind the display screen 8012 in the upper section 8010.

The lower section 8014 of the instrument 11 contains the high voltage power supply and controller for the laser used in the inspection of the capillary electrophoresis. Also in this lower section 8014 are the charge couple device used to sensor the fluorescence and the optics for conveying the light to and from the capillary.

Another embodiment of the instrument is shown in FIGS. 29a, 29b and 29c. The instrument 29-11 is provided within a casing 29-8000. The upper section 29-8002 of the instrument 11 is provided with a door 29-8004. The door 29-8004 is a combination of a top section 29-8006 and front section 29-8008 of the casing 29-8000.

The lower section 29-8010 of the instrument 11 provides the display 29-8012 by means of which the user inputs information into the instrument 11 and receives visual information from the instrument 11.

The window 29-8014 allows for visual inspection of the cartridge used. A series of light bars 29-8016 are used to indicate the extent of progress through the steps involved; the more of the bar which is lit the greater the extent of the step performed.

A stylus 29-8018 is used by the operator to interact with the display 29-8012.

Various control buttons 29-8020 are provided below the screen 29-8012.

The overall dimensions of the instrument are width, W, 419 mm, overall height, OH, 621 mm, depth, D, 405 mm.

The side panel 29-8022 is removable for maintenance purposes.

The embodiment of FIG. 30 shows the door 30-8004 structure more clearly, together with the workspace 30-8024 that is accessed through it. The workspace 30-8024 includes the slot into which the cartridge carrier 30-8026 is inserted. The cartridge carrier 30-8026 is as described elsewhere in this document. The workspace 30-8024 also includes the lane finding apparatus 30-8028.

The cover 30-8030 in the side panel 30-8032 is opened by rotation to allow access to the optics for maintenance purposes.

### Cartridge to Instrument Interface

As described above, once the cartridge 9 is loaded with the sample, the cartridge 9 is loaded into the instrument 11 for the processing to be conducted.

As a first step, the latch 8004 is released and the door 8002 is opened.

To insert the cartridge 9, FIG. 13, the section of the cartridge 9 which bears the PCR chamber 416 is inserted into a slot 8023 between the components which will control the PCR process. These components include the thermoelectric heaters/coolers, Peltier devices 8025, and fans 8027 there for. These components are free to travel to a limited extent to help with the locating of the cartridge 9 within the slot 8023, whilst being forcibly returned to the optimum position after insertion so as to give effective heating/cooling.

The cartridge 9 is provided with a series of recesses which cooperate with dowels extending through the printed circuit board 8008 to accurately register the cartridge 9 relative to the printed circuit board 8008. The dowel arrangement is such that the cartridge 9 cannot be fitted the wrong way round.

Once positioned, the cartridge 9 is provided in a plane which is parallel to the plane of the printed circuit board 8008. Both components have flat surfaces facing one another so as to assist with the good contact needed between them.

The closing of the door 8002 and operation of the latch 8004 applies a compressive force to the cartridge 9 by way of a series of spring loaded pins mounted on the inside surface of the door 8002. This helps hold the cartridge 9 in firm contact with the printed circuit board 8008.

The printed circuit board 8008 is important to the successful operation of the invention. It provides the energy sources for the various components to be driven on the cartridge 9. In effect, the drivers are all provided in the cartridge 9, but the energy sources are provided on the printed circuit board 8008. In this way, the precision operation needed is ensured by the expensive and bespoke electronics and arrangement of the printed circuit board 8008; a reusable component of the instrument. In this way, the cartridge 9 is simple and self-contained. This reduces the complexity of the interface between the two and also removes the risk of contamination of the contents of the cartridge 9. The only transfer between the printed circuit board 8008 and the cartridge 9 is conducted and radiated heat from the heaters and the magnetic field provided by the magnet.

The components provided on the printed circuit board include:
d) The electrical contacts 9000 which connect to the pins of the electrochemical pump electrodes on the cartridge 9. These provide the electrical power, when needed, to operate the electrochemical pumps.
e) The electrical heaters 9002 which are used to apply heat to the valves on the cartridge so as to open or close the valves depending upon their type. These are square areas of resistance heating material which is applied by printing a paste to the desired location. The heating effect is improved if the square block is rotated through 45° relative to the axis of the channel subject to the valve.
f) The magnet 9004 which is advanced into proximity with the cartridge 9 when it is desired to retain the beads and prevent them from moving. The magnet 9004 is retracted away from the cartridge 9 when it is desired to release the beads within the chamber 358.
g) The sensors 9006 are providing feed back and/or verification of the conditions induced by the heaters etc.

### Alternatives for Cartridge to Instrument Interface

If it is necessary to alter or improve the contact between the cartridge and the printed circuit board, there are various options for doing so, including the following:
a) The loading provided by the sprung pins mounted on the door 8002 can be increased. This applies a force to the cartridge 9 and pushes it against the printed circuit board 8008.
b) The cartridge 9 can be mechanically clipped to the printed circuit board 8008, with the clip(s) applying a compressive force.
c) The cartridge 9 can be provided with a compressible substrate mounted on the surface which is intended to contact the printed circuit board. In this way, when then cartridge 9 and printed circuit board 8008 are pushed together, the substrate will provide good all over contact. The substrate can be a solid material, paste or even a liquid. The materials of the substrate, or parts there of, are selected so as to provide maximum thermal conductivity, for instance. Particles, nanoparticles or other materials may be added to alter the properties. The substrate may be protected, prior to use, by a peelable backing
d) As described above, the components (such as heaters etc) are provided in a fixed position on the printed circuit board 8008. This means they move with the printed circuit board 8008. It is possible to provide one or more, and even each of these components with a degree of independent movement. For instance, they may be provided with a sprung mounting on the printed circuit board. In this way, each is able to independently adjust its position, forward and backwards, relative to the cartridge.
e) As shown in FIG. 23, it is possible to provide the section of the cartridge 9 which bears the PCR chamber 416 in opposition to stacked components which will control the PCR process. In this example, the stack includes a first Peltier device 23-01 in contact with the cartridge 9 and in contact with and aligned with a second Peltier device 23-03. The stacking of the devices allows high temperatures, for instance greater than 150° C. to be obtained within the PCR chamber. Such temperatures are beneficial in terms of melting the high melting point wax seals described elsewhere within this document.
f) Alternative forms of heater may be used instead of Peltier effect device. For instance infra red heating devices may be used. The material around the PCR chamber, or a part of that material, may be capable of resistance heating to give the necessary heating for the chamber. Resistance heaters positioned against the cartridge may be used. Microwave heating may be used.

### Alternative Cartridge to Instrument Interface

In the alternative embodiments of the instrument described above in relation to FIGS. 29a, b, c and FIG. 30, the cartridge is not loaded directly into the instrument. Instead, once loaded with the sample, the cartridge 31-01 is loaded into a cartridge carrier 31-03.

The use of the carrier 31-03 means that the cartridge 31-01 and the CE chip can be constructed separately. This allows different material and/or different production tolerances to be used for the different components; a beneficial effect on cost and/or performance and/or the balance between those can thus be provided.

The carrier 31-03 also allows for easy assembly of the required components and their insertion into the instrument in a unitary form. At the same time, the carrier is designed so as to allow separate alignment checking and adjustment for the cartridge and the CE chip so that both are in their correct, optimised position within the instrument.

If desired, the cartridge position can be checked and any alignment adjustment necessary can be made. Before CE starts, a separate check can be made on the alignment of the CE chip, within any adjustments it needs being made before CE starts.

The cartridge carrier 31-03 is illustrated in FIG. 31a. The cartridge carrier 31-03 includes a first support 31-05 and a second support 31-07 which is perpendicular to the first support 31-05.

The first support 31-05 is used to carry the cartridge 31-01. The second support 31-07 is used to carry the capillary electrophoresis, CE, chip; this interaction is described further below.

The prepared cartridge 31-01 is presented with its face 31-09 to the face 31-11 defined by the first support 31-05. An externally threaded screw 31-13 provided at each corner of the first support 31-05 is received into an opposing aperture 31-15 provided at each corner of the cartridge 31-01. Rotation of the screws 31-13 causes them to engage with and enter an internal screw thread provided in the apertures 31-15. Further tightening mounts the cartridge 31-01 on the first support 31-05 and hence the carrier 31-03 in a secure and known position.

The interaction between the cartridge 31-01 and the carrier 31-03 is shown in more detail in FIG. 31b in relation to one of the screws 31-13.

The screw 31-13 is provided with a knurled head 31-17. The threaded engagement occurs between the end 31-19 of the screw 31-13 and the aperture 31-21 in the cartridge 31-01. A jam nut 31-23 in cooperation with a washer 31-25 serves to hold the screw 31-13 on the carrier when not engaged with a cartridge 31-01 The jam nut 31-23. washer 31-25 and sleeve 31-27 serve to prevent over tightening between the carrier 31-03 and the cartridge 31-01.

Rotation of the screw 31-13 pulls the knurled head 31-17 and the cartridge 31-01 closer together. This causes compression of the conical spring 31-29 between the knurled head 31-17 and an abutment surface 31-31 on the first support 31-05. The spring 31-29 assists in ensuring correct alignment during tightening. Once rotation is finished, the first support 31-05 and hence carrier 31-03 is in a known position relative to the cartridge 31-01.

The CE chip 32-31 is inserted into the carrier 32-03 as shown in FIG. 32a. The CE chip 32-31 is slid into a slot. As shown in FIG. 32b, the second support 32-07 provides such a slot 32-33 at either end for receiving the end portions 32-35 of the CE chip 32-31. An incline 32-37 on the lead edge 32-39 of the CE chip 32-31 engages with the end 32-41 of a spring loaded plunger 32-43 and causes it to displace outward, arrow A. Once the recess 32-43 is presented to the end 32-41 of the plunger 32-43, the plunger 32-43 returns, arrow B, and so prevents onward movement of the CE chip 32-31 past the desired position.

Once the cartridge 31-01 and the CE chip 32-31 are inserted into the carrier 31-03, 32-03, the fluid connection between the two is provided by a tube 33-45. The insertion of the cartridge 31-01 into the carrier 31-01 causes the electrophoresis step inlet 28-570 on the cartridge 31-03 (see FIG. 28a) to become connected to the tube 33-45. As shown in FIG. 33a, the tube 33-45 extends upward, parallel to the plane of the cartridge 31-01 and the first support 31-05 through an opening 33-47 in the carrier 31-03. As shown in FIG. 33b, once through the opening 33-47, the tube 33-45 makes a 90° turn into the plane of the second support 31-07 and the CE chip 32-31. The tube 33-45 is accommodated within the second support 31-07 above the CE chip 32-31. A further 90° turn leads the tube 33-45 into the CE chip 32-31. The remaining fluid transport is handled within the CE chip 32-31 itself, as described elsewhere in this document.

After insertion of the cartridge 31-01 and the CE chip 32-31 into the carrier 31-03, as described above, the carrier 31-03 is ready for insertion.

As a first step, the door 34-8004 is opened, FIG. 34a, to expose the workspace 34-8024. The work space 34-8024 includes the slot 34-47 that the carrier 34-03 is inserted into.

The carrier 34-03 is inserted into the slot 34-47 until the second support 34-07 comes to rest on the surface 34-49 of the workspace 34-8024. The cooperation of the carrier 34-03 with the slot 34-47 ensures the correct general positioning of the cartridge 34-01 with respect to the instrument, both in terms of lateral and vertical positioning; FIG. 34b.

Insertion in this way provides the section of the cartridge which bears the PCR chamber between the components which will control the PCR process; as described further below.

Once inserted, the door 34-8004 is closed. The closing of the door 34-8004 triggers various actions based upon contact between the closed door 34-8004 and casing. The clamping of the cartridge to the PCB, the positioning of the CE chip on the CE chip heater board, the introduction of the electrical contacts to the pins provided on the CE chip, the introduction of the electrical contacts to the pins providing the conduction path to the electrodes in the electrochemical pumps are all triggered in this way. The closure of the door 34-8004 is also used to turnoff the interlock for various safety systems within the instrument. The interlock prevents, for instance, the laser being active with the door or any other opening in the instrument's casing being open. a similar principle applies to the power supplies within the instrument.

As with other embodiments, it is important to provide effective and accurate contact between the cartridge and the instrument interface. In FIGS. 35a, b and c the provision of the contact is illustrated.

FIG. 35a shows the carrier 35-03 in position in the slot 35-47. In the insertion position, as shown, the arrangement provides for a gap 35-51 between the face 35-53 of the cartridge 35-01 which opposes the face 35-55 of the printed circuit board 35-57 of the instrument.

In the next step, FIG. 35b, the cartridge 35-01 is moved into the use position. A platen 35-59 is moved, direction of arrows, by an actuator, not shown. This causes the cartridge 35-01 to be brought into full contact with the PCB 35-57. The movement is such that the conical spring 35-29 is further compressed. During this movement, a series of rods which extend through the PCB 35-37 enter various holes (27-13 in FIG. 27) and so ensure that the alignment between the cartridge and the PCB is correct in that orientation too.

When the use of the cartridge 35-01 has finished, then the force applied to the platen 35-59 by the actuator is released. As a result, the carrier 35-03 is returned to the insertion position by return springs, not shown. The release causes the conical springs 35-29 to pull the cartridge 35-01 back into position inside the carrier 35-03, FIG. 35c. The carrier 35-03 can then be removed by lifting it out of the slot 35-47, taking with it the cartridge 35-01.

The face to face contact between the cartridge and the PCB provides the majority of the interactions between the cartridge and the instrument, for instance, heating for valve control, sensor etc. The contact between the PCR chamber and its temperature cyclers are provided through further components, however; see FIG. 36a, b, c and d.

In FIG. 36a, the cartridge 36-01 is shown inserted into the slot provided in the instrument. Once inserted, the section of the cartridge 36-01 bearing the PCR chamber is positioned between a pair of calipers 36-100. The PCB is cut away at this location so as to not be in the way of the Peltier effect devices 36-102, 36-108 and pair of calipers 36-100. The calipers 36-100 are floating such that they do no interfere with the contact sought between the cartridge 36-01 and the PCB during the movement from the insertion position to the use position.

The front caliper 36-100a is provided with a Peltier effect device 36-102 mounted on a support 36-104 which is capable of reciprocating movement, arrow C, under the control of actuator 36-106. The actuator 36-106 is also mounted on the pair of calipers 36-100.

The back caliper 36-100b is provided with a second Peltier effect device 36-108 mounted fixedly on the caliper 36-100b. The second Peltier effect device 36-108 is provided in opposition to the Peltier effect device 36-102.

In the open position shown in FIG. 36c, such as is provided with the cartridge in the insertion position, the distance between the opposing faces 36-110, 36-112 of the Peltier effect device 36-102 and the second Peltier effect device 36-108 is more than the thickness of that section of the cartridge 36-01 and more than the thickness of the carrier 36-03 which passes between the pair of calipers 36-100 during insertion of the carrier 36-03.

In the closed position shown in FIG. 36d, such as is provided during the amplification step, the distance is reduced. This is achieved by the actuator 36-106 moving the Peltier effect device 36-102 on the front caliper 36-100a towards the cartridge 36-01 and towards the opposing second Peltier effect device 36-100b. This actuation, combined with the floating nature of the pair of calipers 36-100 brings both of the Peltier effect devices into firm contact with the cartridge 36-01 on opposing sides thereof. They are now in position to provide the necessary heating and/or cooling for the PCR step.

Thermocouples to sense the temperatures applied, and potentially to be used to control the temperatures applied, are provided in close proximity with the Peltier effect devices, embedded in copper shims, bonded to the Peltier effect devices.

Before the carrier 36-03 is removed, the actuator 36-106 returns the Peltier effect devices 36-100 to the open position.

In addition to the carrier allowing for relative movement of the cartridge to ensure correct positioning with respect to the PCB, the carrier also allows for totally independent relative movement of the CE chip. This is importing in ensuring correct positioning of the CE chip for the CE step. This is achieved by the structure and operation shown in FIG. 37a and b.

As the carrier 37-03 with the CE chip 37-31 in it is inserted into the slot in the instrument, the second support 37-07 approaches the work surface 37-49. The work surface 37-49 carries a CE chip board heater 37-100 in the form of a planar surface. this is surrounded by a raised surface 37-102 which provides a nest for the CE chip 37-31 once positioned.

Projecting pins 37-104 on the work surface 37-49 enter apertures 37-106 provided in the second support 37-07 of the carrier 37-03; FIG. 37a. In FIG. 37b, the top part of the second support 37-07 is shown cut away so that the full extent of the CE chip 37-31 can be seen. The apertures 37-106 in the second support 37-07 align with the slot 37-108 which receives the end portions 37-108, 37-110 of the CE chip 37-31. As a result, the end portions 37-108, 37-110 are also provided with through apertures 37-112a, 37-112b. The projecting pins 37-104 thus pass through these apertures 36-112a, 36-112b too as the carrier 37-03 approaches the work surface 37-49.

The conical ends of the pins 37-104 mean that they enter the apertures 37-106, 37-112a, b, even where there is potential misalignment. The fuller diameter parts of the pins 37-104 encourage the CE chip 37-31 into the correct position. The CE chip 37-31 is centred to the CE chip board heater 37-100 as a result. The CE chip heater board 37-100 and raised surface 37-102 can be seen clearly in FIG. 38.

### Electrophoresis Components

### 1) Optics

In the electrophoresis step 206, at the detection location 628, light from a laser 800 is focussed to be incident upon the fluorescent dye associated with a DNA element to make it detectable.

A different dye is used for each different DNA element type; a type is generally associated with a given locus.

To get good sensitivity, it is important for the incident light to be of sufficient intensity for the detectors to receive sufficient light to be sensitive to the emitted fluorescent light, but for the intensity not to be so high as to give rise to photobleaching of the dyes. To provide for this, the following arrangement is used; FIG. 14.

The light source is a compact laser 900 which is mounted on a heat sink 902. The laser 900 is a Cobolt Calypso laser (from Cobolt AB, Kraftriken 8, SE-104 05, Stockholm, Sweden) and emits at 491 nm with a maximum power of 50 mW. The light emitted by the laser 900 is fed to a fibre coupler 904 (09 LFC 001, f=3.5 mm from Melles Griot, 2051 Palomar Airport Road, 200, Carlsbad, Calif. 92011, USA) and hence into an patch cable assembly (M31L01, from Thorlabs, 435 Route 206 North, Newton, N.J., 07860, USA) and optical fibre 906 (GIF625, dia 62.5 μm, NA=0.275 from Thorlabs, 435 Route 206 North, Newton, N.J., 07860, USA).

The use of the optical fibre 906 is beneficial as it safely controls the laser light direction, enables the laser light to be easily conveyed to the position of use and enables mechanical stability to be provided within the overall system. At the end of the optical fibre 906 a power of up to 45.32 mW is still observed.

The laser light then passes through a collimator 908 (F230FC-A, F=4.5 mm, NA=0.55, from Thorlabs) and a logpass filter with a sharp cut-off wavelength, EM filter (Omega Optical XF3093, T50=515 nm) before reaching the spot mirror 910.

The spot mirror 910 is used to both direct the laser light to the detection location 628 of the capillary and to transmit, anisotropically and without filtering, the fluorescent light received there from to the detector unit. It is angled at 45° to the beam of laser light. To do this, the reflector 910 consists of a 25 mm round glass disc which transmits all light from <80 above 380 nm. An ellipse, 2 mm long by 1 mm wide, is provided at the centre of the reflector 910 (so as to present an effective 1 mm circular mirror), formed of a highly reflective mirror layer deposited there (reflectivity of 99.99%).

Before reaching the detection location 628, the laser light passes through a focussing lens 912. This can be a microscope optic or other such adjustable focussing lens. Such optics are useful as they introduce no optical aberrations to the light, shape the beam for application to the detection location 628 and don't give any selective loss of light colours. The power reaching the detection location 628 is over 27.40 mW.

The fluorescent light is effectively scattered from the dye in the capillary 616 in all directions. For the fluorescence light to reach the detector unit, that light needs to hit the spot mirror 910 at a location outside of the glass spot. If it does so, the light is transmitted into the detector unit 914.

The detector unit 914 includes a slit in front of a spectrometer to obtain diffraction-limited incident light, the spectrometer provided with a diffraction grating and a lens 918 (LA1608A plano convex, f=50 mm, D=25 mm, with anti-reflective coating within 350-650 nm, made of BK7 glass, Thorlabs Inc), to direct the light to the charge coupled device 916. The CCD 916 has spectroscopic abilities.

The CCD 916 generates the signals which are then used to generate the electropherogram, an example of which is shown in FIG. 15

Using such an approach, a sensitivity approaching that of laboratory style electrophoresis instruments can be reached. The instrument is able to detect down to the presence of 2.5 pM of fluorescein dye at pH 7.

In an alternative approach, certain problems with the stability of the fibre optics can be avoided by providing an open beam approach to delivering the light from the laser to the channel.

An alternative embodiment of the optics is shown in the cut away perspective view of FIG. 39. The instrument casing 39-01 provides various mounts for the optics. The light is generated by the laser head 39-03 operated under control by the laser controller 39-05. The light enters the optics 39-07 and is directed at the channel in the CE chip, not shown, mounted in the CE chip heater board 39-09.

The return light enters the optics 39-07 and is directed back to the spectrometer 39-11 and CCD camera 39-13. Above the CE chip heater board 39-09 is the chip alignment structure 39-15 which is described further below.

### 2) Calibration and Verification for Optics

When first using the optics for detecting the electrophoresis results, and periodically thereafter, it is beneficial to ensure that the optics are properly calibrated to the capillary 616 at the detection location 628 in the electrophoresis cartridge section. This ensures best transmission of the excitation light into the detection location 628, best recovery of the fluorescence light from the dyes encountered at the detection location 628 and the performance of the detection at the detection location 628 (and hence at the correct distance from the point at which the sample is injected).

To achieve these aims, the electrophoresis cartridge section is provided with various aids. These are intended to allow automated verification and calibration of the position by the instrument 11.

Firstly, a fixed marker is provided on the electrophoresis cartridge section, a known distance along the capillary 616 and a known distance perpendicular to the capillary 616, from the detection location 628. When the laser light is incident upon the fixed marker, a response is detected by the CCD 916. The position of the incident laser light is thus known. The incident position of the laser light along the capillary is thus correct. The known distance of the fixed marker from the detection location 628, perpendicular to the capillary 616 can then be used to adjust the position at which the laser light is incident so as to correspond with the detection location 628. X and Y axis verification of the incident laser light position corresponding with the detection location 628 is thus provided. The marker could be a physical mark (for instance etched) on the cartridge and/or a coloured mark (for instance a dye) and/or a quantum dot.

To provide for the verification on the Z axis, the working distance between the lens and the capillary 616, a known source, with a known characteristic is provided on the electrophoresis cartridge section at a known Z axis distance relative to the correct Z axis distance of the capillary 616. By adjusting the focus of the lens so as to maximise the response by the CCD 916, the correct working distance for the known source is established. An adjustment can then be made to reflect the relative working distance for the known source relative to the capillary 616. Ideally, these are in the same plane at the same working distance so as to allow the known to provide direct verification for the Z axis position relative to the capillary 616.

As an alternative means of verification on the position, it is possible to use the marker for the X axis and then use variation in transmission to check the Y axis position. Thus a marker is used to determine the correction position along the axis of the capillary 616. The adjustment can then scan in the Y axial direction are use the CCD (or another detector) to consider the variation with position. The reflected signal will be constant at a level when the laser light is incident on the cartridge away from the capillary. When incident light traverses the capillary 616, then the signal will vary in a predictable manner, so allowing the position to be set subsequently at the position corresponding to the middle of the capillary 616 in the signal. To assist in this, it is possible to introduce a polariser insert for the calibration part of the process so as to increase the observed variation in the signal. The polariser is removed before the actual electrophoresis results collection starts. The effect whose variation is detected can arise from the capillary 616 itself, a marker at a known distance from the capillary 616 or a material present in the capillary 616 (for instance, a dye labelled component provided as part of a sizing standard, whose mobility is higher than the other elements of the size standard or unknown elements).

The FIG. 39 and FIG. 40a, b and c embodiment shows the alignment structure 39-15 and its operation.

The alignment structure 39-15 is in the form of a swing arm 40-100 which can be pivoted relative to the casing 40-102 under the power of an actuator contained within the swing arm 40-100. The other end of the swing arm 40-100 is provided with a camera 40-104.

In the stowed position, FIG. 40b, the swing arm is positioned in contact with a hard stop 40-106 mounted on the casing 40-102 too. In the check position, FIG. 40c, the actuator has caused the swing arm 40-100 to swing away from the casing 40-102 and so position the camera 40-106 over the channel 40-108 in the CE chip 40-31.

In the use position, triggered by the operator, a laser is activated and this creates a diffraction pattern which can be seen on the camera display. The adjustment for the CE chip position is used to move the CE chip until the diffraction pattern indicates that the middle of the channel has been located. The alignment of the channel with the optics used in the analysis is thus provided. The camera can also be used to achieve focussing of the system in the Z axis adjustment.

### 3) Electrophoresis Environment Control

For the necessary resolution to be obtained in the electrophoresis step 206, the temperature of the capillary 616 and its contents need to be carefully controlled at the optimum temperature. In the present embodiment, the electrophoresis cartridge section is in contact with a thermally conductive block, with a series of resistance heaters provided on the opposing side of the block. These are provided with controllers and are capable of maintaining the temperature of the electrophoresis cartridge section at the optimum temperature +/−0.3° C.

In addition, the cavity that the electrophoresis cartridge section is provided in is thermostatically controlled at the optimum temperature. This reduces still further temperature variation before, during and after use.

The use of a CE chip heating bed, and raised surface around it, is beneficial in controlling the temperature within the CE chip. The nest so formed ensures consistent positioning and good contact.

### 4) Use of LED's as Light Source

FIG. 16 depicts a schematic of an example of a system for detecting fluorescence. The system includes light emitting diodes (LEDs), e.g., high power cyan LEDs, to provide excitation wavelength light to detect dyes combined with biological samples. The system also includes a bifurcated optical fibre assembly made, e.g., from high transmission fused-silica cores with high numerical apertures (NAs), e.g., NA=0.22. The LED excitation system described herein can be applied for DNA detection in capillary electrophoresis systems in mobile analytical units. The compactness and light weight of the LED system enables automating assays for nucleic acid studies. Using the compact and light weight system allows creating bench-top analysis systems that can be used both in the laboratory and in the field.

In some implementations, two LEDs are assembled in parallel and supplied with a stabilized DC voltage of 3.6 V. The current passing through the LED assembly is 1.8 A. The junction is maintained at 15±1° C. by a Proportional-Integrative-Derivative (PID) control loop (Model TE-36-25 from T.E. Technology, Inc.) acting on two 13×13 mm thermoelectric modules. To save power, and space, two Peltiers modules are controlled in parallel and the thermocouple sensor is placed on only one of them assuming that, by construction symmetry, they both behave similarly. An aluminium heat sink and a fan (12 V DC) complete the cooling module. This module extends the lifetime of the LEDs by two orders of magnitude. Without cooling the junction, the supplied current is 2.7 A.

The first step of collimation is the use of an acrylic-molded lens from Lumiled, which collimates the emitted light to a 15° cone half-angle (NA˜n sin(θ_{1/2})˜0.26). The light is then focused onto a plano-convex lens (f=35 mm, D=25 mm; NA˜D/2f˜0.36). NA_{LED}<NAₗₑₙₛ or the numerical apertures are matched. The distance between the apex of the lens and the plane of the collimator, Lₘₐₓ, is adjusted by a micrometer screw to maximize the power read by a calibrated silicon photodiode sensor. The value obtained (25 mm) is only close to the focal length f since the collimated LED is not a point source. The light beam is then refocused onto a collimation package assembled around an aspheric lens (f=10 mm, D=5 mm; NA˜D/2f˜0.25, Ocean Optics Ltd) within an anodized aluminum lens tube of length l=30 mm. Each LED is thus coupled into one arm of a 2 m-long bifurcated silica core (Ø=600 μm, NA=0.22) optical fibre assembly (attenuation: 0.013 dB/m at 505 nm—relative transmission: 82% (arm 1) and 87% (arm 2)).

Table 1 illustrates a power optimization of the system depicted in FIG. 16. The power at 505 nm, P505, is read by the silicon photodiode while the distance between the LED collimator and the lens surface (Lₘₐₓ), the lens geometry, and the lens tube length (l) are changed. Only one arm of the bifurcated fibre is used.

TABLE 1

| Lens | I | Lmax | Psos |
|---|---|---|---|
| Hemispherical | 3 cm | 20 mm | 225.2 μW |
| Hemispherical | 5 cm | 18 mm | 200.4 μW |
| Hemispherical | 8 cm | 19 mm | 222.8 μW |
| Cylindrical | 3 cm | 9 mm | 170.9 μW |
| Cylindrical | 5 cm | 9 mm | 164.1 μW |
| Plano-convex | 3 cm | 16 mm | 220.9 μW |
| Plano-convex | 5 cm | 15 mm | 204.1 μW |
| Plano-convex | 8 cm | 15 mm | 173.7 μW |
| None | No'ne | 12 mm | 187.4 μW |

For the bias values described above, when both arms of the fibre are used, the power at 505 nm read by the photodiode is 820 μW.

FIG. 17 is a plot of LED spectrum, light reflected, and residual LED light over a range of wavelengths (nm). FIG. 17 illustrates an LED spectrum obtained in the cooled CCD (diodes: Ug=2.0 V; I=0.3 A; T=15° C.), calculated light reflected by the dichroic mirror, and residual LED light after the emitter. The insert shows the transmission curves of the dichroic and emitter. The plot indicates that there is a loss of power when the incident light is reflected onto the sample. Additionally, light is red-shifted by 20 nm, which causes some of the LED light to interfere with the carboxyfluorescein dyes. The choice of available emitters and dichroic mirrors is limited by the dyes chosen to label the migrating DNA strands.

FIG. 18 is a plot of power of the LED-module over time. During a CE experiment, it is crucial to reduce the fluctuations of the power of the light source within less than 1%. FIG. 18 shows an example of the power recorded by the silicon photodiode (Probe S130A, Thorlabs) using the internal calibration function to record the power emitted by the fiber-LED assembly at 505 nm over time. The diodes are supplied with a 3.4 V DC voltage corresponding to a current of 1.4 A while the junction is maintained at 15±1° C. The room is maintained at a temperature of 22° C. (R.H.=24%). The plot illustrates a temporal power evolution of the LED-module. The lines mark regimes where the power drops, e.g., by 4.8 nW/s, 11.6 nW/s, and 5.0 nW/s. Overall, the power drops by about 1.95 μW over 5 min, i.e. 0.48%.

FIG. 19 is an illustration showing beam shape and size after the sample objective as measured by the laser camera. The asymmetry observed is due to imperfections occurring when the two fibre arms are fused because of the large core diameter of the fibre, mismatches between the LED-to-LED and the fiber-to-fibre distances, and tilt in the optical elements. In the results reported in the next section, the situation corresponding to the single-spot will be used. One method includes adjusting all the optics to obtain the maximum power at the merged end of the bifurcated fibre. This can yield a misshapen light beam as the core size of each arm is large (multimode fibre). To characterize the beam shape and size after the microscope objective, i.e. at the entrance of the microchip, a Coherent Lasercam II ½ camera was placed on an {x,y,z} translation stage equipped with micrometer precision positioners and equipped with a Leica HCX PL FLUOTAR (40×, NA=0.75, WD=0.40 mm) and adjustable filters. The objective was brought within ˜8 mm of the Olympus LUCPLFLN (20×, NA=0.45, WD=6.6-7.8 mm) mounted on the CE setup. This allowed directly imaging the beam coming out of the fiber-LED assembly via the CE setup. The micrometer positioners allowed measuring the dimension of the beam with a precision of 10 μm by moving the camera from one spot of the obtained beam profile image to another and reporting the traveled distance. The power can be maximized by adjusting each optical collimation element (P=1.6 mW at 505 nm) (A) or the collimation elements can be adjusted to give one single spot (P=1.0 mW at 505 nm) (B).

The system was employed for both static and dynamic fluorescence measurements. For the static fluorescence measurements, a 1 μM fluorescein, 6-FAM or rhodamine B solution is loaded into the microchannel by using a standard laboratory vacuum line (13 PSI (0.88 atm) depression) to pull the solution through the channel via 2-mm-diameter access holes. The glass microchannel is anisotropically etched with fluorhydric acid (HF) in Schott Borofloat® low-fluorescence glass (CE chip X8050, Micronit, B.V., The Netherlands). It is semi-elliptic with a width of 50 μm, a depth of 20 μm and a length of 85 mm. The plastic microchannels are hot-embossed into a 1.1-mm-thick cyclic olefin copolymer (COC) sheet at ˜160° C. from a reactive-ion etched Si(100) master. The channel section is tapered with a 25° taper angle and has a width of 60 μm (top) and 39 μm (bottom), a depth of 20μ and a length of 85 mm. Glass capillaries that are 1-cm-long (inner diameter: 4 mm) borosilicate are epoxy-glued onto the access holes to act as reservoirs (or wells). All solutions are filtered with a nylon membrane (pore diameter: 0.2-μm) to remove small particles that will clog the channel.

The loaded chip is placed on the CE setup and the focus of the 63× sample objective is aligned with the bottom of the channel. The emitted fluorescent light is gathered onto the 26.6 mm×6.7 mm (1024×255 pixels) array of the thermoelectrically cooled Andor CCD. The processed signal is vertically binned from the software-restricted central rows irradiated by the light focused onto the spectrometer entrance slit. The CCD is cooled down to −50° C. to reduce the binned dark counts to 270 while the exposure time is 0.05 s.

FIGS. 21A and 21B are plots of CCD signal v/s wavelengths. The plots indicate the vertically-binned signal from a 1 μM 6-FAM solution loaded into a glass microchannel (A) and a 1 μM fluorescein solution loaded into a plastic COC channel (B). The counts from the same microchannel filled with water are subtracted to take into account the autofluorescence of the glass or plastic microdevice. The power emitted from the system is 0.98 mW and 1.03 mW at 505 nm for glass and COC, respectively. This is obtained by supplying the two LEDs (placed in series) with a constant current of 0.74 A, which corresponds to a voltage of 7.0 V. Due to the choice of filters (emitter cut-on: T50 at 535 nm), only the tail of the fluorophore emission is observed (fluorescein: λ^{em}ₘₐₓ=513 at pH=13, 6-FAM: λ^{em}ₘₐₓ=517 at pH=9. The signal-to-noise ratio is 87 for 1 μM 6-FAM in glass and 36 for 1 μM fluorescein in COC. The SNR is lower in glass because 6-FAM is known to photobleach faster than fluorescein. The detection limit parameters for glass and plastic CE microdevices are summarized in Table 2.

TABLE 2

| Device | | Power at | Maximum | signal-to- |
|---|---|---|---|---|
| material | Fluorophore | 505 nm | counts | noise ratio |
| Glass | 1 uM 6-FAM | 0.98 mW | 720 | 36 |
| COC | 1 uM fluorescein | 1.03 mW | 1750 | 87 |

For dynamic fluorescence measurements, glass microchannels are loaded with reagents similar to the reagents for the static measurement testing, but a first sequence of reagents are flushed through the microdevice to reduce the effect of the electroosmotic flow (EOF) that opposes the electrophoretic flow and results in peak distortion from a Gaussian shape and therefore loss of resolution. EOF arises from the re-equilibration of the electrical double layer arising from the surface charge of the microchannel walls after the perturbation caused by the migrating charges under the electric field. The EOF can be efficiently controlled by using a coating polymer matrix such-as poly-N-hydroxyethylacrylamide (pHEA) dissolved in water at 0.1% w/v.

The DNA fragments are separated by electrophoretically migrating within a sieving polymer matrix such as POP-5™ (Applied Biosystems, Inc.), a mixture of polyacrylamides in an appropriate buffer, according to their size and interactions with the polymer network. After the pHEA coating has been applied, IX A.C.E.™ buffer (Amresco, Inc.) is flushed into the channel by vacuum followed by POP-5™. A 1 μM solution of a poly-adenine oligonucleotide labeled with 6-FAM is placed in the sample well and will be electrokinetically injected in the separation channel via a cross-injection geometry. 1X A.C.E.™ buffer is placed in the sample waste, buffer waste, and waste wells to ensure ionic conductivity in the whole device.

FIG. 21 is a plot of CCD signal v/s time for dynamic fluorescence measurements. The plot indicates fully binned CCD signal showing the peak corresponding to the elution of the 1 μM oligonucleotide (elution time, tₑₗ=77 s) detected by the optical module. The nature of the peak is confirmed by the spectrum obtained in the CCD at t=77 s. It is similar to the peak shown in FIG. 20A. The signal-to-noise ratio of 10 can be improved by uniformly heating the chip to 50° C. The plot shows the result of the migration of the oligonucleotide while the LED-fibre assembly delivers about 980 μW at 505 nm. The two LEDs, placed in parallel, are supplied with 3.9 V (I=1.9 A) while the junction is kept at 15° C. The migration field in the separation channel is 110 V/cm.

In this manner, an optical excitation module capable of visualizing a 1 μM oligonucleotide migrating in a glass microchannel loaded with a sieving matrix is assembled and tested. The output fibre beam size and divergence, the power distribution in the beam exiting the fibre assembly as well as the output power stability over time approach the specifications of existing LIF setups. A modified epifluorescence microscope arrangement is used in conjunction with a lightweight compact fixed spectrograph built around ion-etched grating and aligned with a cooled Charge-Coupled Device (CCD) camera for added sensitivity. Fluorescent dyes such as fluorescein, 6-carboxyfluorescein (6-FAM) and rhodamine B can be detected in conventional plastic (cyclic olefin copolymer) and glass microchannels at submicromolar levels. A migrating single-stranded oligonucleotide DNA fragment (10-mer) labeled with 6-FAM can also be detected with high signal-to-noise ratio when electrophoretically migrated in the microchannels at 100 V/cm. LEDs operated in conjunction with Peltier elements controlled by a Proportional Integrative Derivative (PID) module can be used to replace bulky, expensive and power-consuming Argon ion lasers conventionally used in Laser Induced Fluorescence (LIF) Capillary Electrophoresis (CE) experiments. The LEDs in the system can be HP803-CN obtained from Roithner LaserTechnik GmBH or Luxeon Star series from Philips Lumiled Lighting Company that offer LEDs emitting at 505±15 nm with a full-width at half maximum of 20 nm. The LEDs are available with a Lambertian profile with a half-cone angle of 75°, which is not suited for microchip applications. However, these are high power LEDs with a nominal radiometric output power of 45 or 80 mW. When properly collimated, the available power becomes relevant to applications of DNA detection by CE.

While this specification contains many specifics, these should not be construed as limitations on the scope of the disclosure or of what may be claimed, but rather as descriptions of features specific to particular implementations of the disclosure. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the disclosure have been described. Other implementations are within the scope of the following claims. For example, the actions recited in the claims can be performed in a different order and still achieve desirable results. In some implementations, the sharpness of the cut-on edge of the dichroic mirror can be improved and the lower wavelength T₅₀ can be shifted to a lower wavelength to improve the signal-to-noise ratio. In some implementations, the diodes can be operated in a pulsed AC mode where the “on” time is synchronized with the frame acquisition of the CCD camera, thereby also extending the lifetime of the LEDs. In some implementation, a customized LED array can be used that does not have the mold that yields divergent light. In some implementations, the collimation parts can be embedded in a rigid casing made, e.g., from black anodized aluminum.

In some implementations, the LED-based detection system described in this disclosure can be used as the micro fluidic electrophoresis system that is described in the attachment, which is enclosed as part of the present disclosure.

### 5) Size Standards

The size standards used in the invention are beneficially stored within the formamide pump liquid.

The size standards may be provided according to the form detailed in International Patent Application no PCT/GB2009/002186, the contents of which are incorporated herein by reference, particularly with respect to the provision of and use of size standards which operate within a single CE channel, together with the sample being considered.

### Instrument Performance

The result of the above embodiment is the provision of an instrument, cartridge and operating method which provides quick, reliable sample analysis, whilst doing so at a wide variety of locations and when operated by a wide variety of people.

By way of abilities are performance, the invention provides a fully integrated instrument capable of performing extraction, PCR, electrophoresis and analysis, whilst requiring minimal training and/or intervention by the user. In its optimum form, a fully automated system from start to finish is provided, the user simply needing to load the cartridge into the instrument and start it.

The modular nature of the instrument allows for upgrading of one or more modules without impact on the other modules. The data output format has been carefully selected to allow the analysis of the data outputted by a variety of existing analysis software applications, such is I³ of Forensic Science Service Limited, and future software applications.

The end result of the analysis may be a profile for the sample and/or an indication of a match between the sample and a database recorded sample and/or other interpretation based data.

The use of a single cartridge type to handle a wide variety of sample from a wide variety of sources is beneficial. The methodology is able to handle samples originating from buccal swabs, cotton and other soft swabs, aqueous samples, clothing samples, cigarette butts, chewing gum and the like.

The methodology is also able to separate the useful DNA from residual cellular material, PCR inhibitors (such as ethanol, indigo etc) and chemical inhibitors.

The instrument is fully portable and so can be used in a wide variety of locations. The fully sealed and protected nature of the cartridge means that contamination is not a risk, even where the instrument is used outside of laboratory standard conditions. The instrument operates off a standard mains power supply, 110-240V, 50 Hz, using a conventional electric plug.

With respect to the overall time, from the sample receiving step 202, to the transmission away from the instrument in the data communication step 210, the embodiment described provides this process in a time period of 141 minutes. That time period can be reduced, including by the options and variables set out in the following paragraphs.

With respect to the sample receiving step 2002, the embodiment described provides this step in a time period of 2 minutes. Time periods of between 20 seconds and 5 minutes are easily achievable, depending upon the loading methodology used and the number of reagents or samples that need to be loaded.

With respect to the sample preparation step 202, the embodiment described provides this step in a time period of 24 minutes. That time period can be reduced by shortening the residence in one or more of the chambers, for instance the incubation chamber 358, and/or by reducing the time separation between a valve being activated and reliance on the outcome of the activation and/or by reducing the washing and/or elution volumes used. Time periods of between 15 to 30 minutes are easily achievable.

With respect to the sample amplification step 204, the embodiment described provides this step in a time period of 80 minutes. That time period can be reduced by shortening the number of cycles used, the duration of one or more parts of a cycle and the time period after introduction to the chamber and before PCR starts and/or after PCR finishes and before the sample is removed to the next stage. Again, the time separation between a valve being activated and reliance on the outcome of the activation is of significance. Time periods of between 60 to 120 minutes are easily achievable.

With respect to the electrophoresis step 206, the embodiment described provides this step in a time period of 15 minutes. That time period can be reduced by the use of higher voltages and/or faster migration media in the capillary and/or reductions in the sample introduction time. Time periods of between 1 to 60 minutes are easily achievable. This functionality is achieved in an instrument weighing less than 10 kg and occupying a footprint of less than 0.1 m².

### Instrument Fields of Use

The structures and method discussed above are useful in the consideration of a wide variety of samples, over and above forensic samples. For instance, they can be used: the consideration of marker targets, diagnostic assays, disease markers, biobanking applications, STR based targets in transplants, identification of drug resistant microorganisms, blood testing, mutation detection, DNA sequencing and the like. Food analysis, pharmogenetics and pharmogenomics are also areas of use. A wide variety of uses in the medical and/or biotech field can make use of the invention.

The invention is also applicable in situations where familial relationships need to be determined from DNA, for instance paternity testing. Pedigree testing in animals is a further example.

The use of the invention in border control, security, customs situations and other governmental type uses is beneficial.

1. A method of analysing a sample, the method comprising: applying one or more process steps to the sample; and obtaining one or more results from the method; wherein an instrument provides some of a set of process steps and/or sample processors and one or more process steps and/or sample processors are provided separately from the instrument.

2. A method according to claim 1, wherein the one or more process steps and/or sample processors provided separately from the instrument include one or more of: a sample receiving step and/or sample preparation step and/or sample extraction step and/or sample retention step and/or purification step and/or washing step and/or elution step.

3. A method according to claim 1, wherein the one or more process steps and/or sample processors provided by the instrument include one or more of: a further sample receiving step and/or amplification step and/or PCR step and/or denaturing step and/or investigation step and/or detection step and/or electrophoresis step and/or analysis step and/or results output step.

4. A method according to claim 1, wherein the one or more process steps and/or sample processors provided separately from the instrument include a sample receiving step and sample preparation step and sample extraction step.

5. A method according to claim 1, wherein the one or more process steps and/or sample processors provided by the instrument include a sample receiving step and amplification step and denaturing step and detection step.

6. A method according to claim 1, wherein the one or more process steps and/or sample processors provided separately from the instrument include a sample receiving step and sample preparation step and sample extraction step and sample retention step and/or purification step and washing step and elution step.

7. A method according to claim 1, wherein the one or more process steps and/or sample processors provided by the instrument are an integrated set, and wherein the one or more process steps and/or sample processors provided by the instrument include a sample receiving step and amplification step and denaturing step and investigation step and detection step and results analysis step and results output step.

8. A method according to claim 1 wherein the one or more process steps and/or sample processors provided by the instrument are provided by a device in the form of a cartridge.

9. A method according to claim 1, wherein the one or more process steps and/or sample processors provided separately from the instrument include one or more of: a sample collection step and/or a sample transportation step and/or sample storage step and/or a sample receiving step and/or sample preparation step and/or sample extraction step and/or sample retention step and/or purification step and/or washing step and/or elution step.

10. A method according to claim 1 wherein the sample is received from one or more of: a solid matrix, a solid matrix containing fibres, paper, a swab, a buccal swab, a cotton swab, a soft swab, a solution, a suspension, an item of clothing, an item placed in the mouth, a cigarette or piece thereof, chewing gum, one or more hairs, a bone sample, a tissue sample or saliva.

11. A method according to claim 1 wherein the sample receiving step and/or sample preparation step and/or sample extraction step and/or sample retention step and/or purification step and/or washing step and/or elution step are provided with one or more of:
a. one or more chemicals and/or reagents;
b. one or more chemicals and/or reagents to buffer the sample during one or more steps;
c. one or more chemicals and/or reagents to alter the sample during one or more steps;
d. one or more chemicals and/or reagents to detach and/or remove and/or release the sample or a part thereof from the form wherein the sample is received from the previous step;
e. one or more conditions which differ from ambient conditions;
f. one or more conditions which differ from ambient conditions in terms of the temperature compared with ambient and/or the level of gravity compared with ambient;
g. one or more conditions which differ from ambient conditions and detach and/or remove and/or release the sample or a part thereof from the form wherein the sample is received from the previous step.

12. A method according to claim 1 wherein the method further comprises a further sample receiving step which comprises the transfer of a sample from outside the device and/or instrument to inside the device and/or instrument.

13. A method according to claim 12 wherein the further sample receiving step comprises receiving the sample from a transport device and/or from a storage device and/or comprises the transfer of the sample to a channel or chamber within the device.

14. A system for analysing a sample, the system comprising an instrument for analysing a sample, the instrument comprising: a device having one or more sample processors; electronics for operating the sample processors; wherein the system comprises the instrument providing one or more of a set of process steps and/or sample processors and wherein one or more process steps and/or sample processors are provided separately from the instrument.
